(19) United States
(12) Patent Application Publication
(10) Pub. No.: US 2011/0190351 A1
(43) Pub. Date: Aug. 4, 2011

(54) Benzoimidazole Derivatives and Glycogen Synthase Kinase-3 Beta Inhibitors Containing the Same

(75) Inventors: Mitsuaki Ohtani, Kanagawa (JP); Yo Matsuo, Kanagawa (JP); Yingfu Li, Clifton, NY (US); Joel R. Walker, Schenectady, NY (US); David M. Jenkins, Cohoes, NY (US); Feryan Ahmed, Latham, NY (US); Ryuji Ohsawa, Kanagawa (JP); Shoji Hisada, Kanagawa (JP)

(73) Assignee: Oncotherapy Science, Inc., Kanagawa (JP)

(21) Appl. No.: 13/056,591

(22) PCT Filed: Jul. 30, 2009
(86) PCT No.: PCT/US09/52225

§ 371 Date: Apr. 21, 2011

Related U.S. Application Data
(60) Provisional application No. 61/084,770, filed on Jul. 30, 2008.

(51) Int. Cl. A61K 31/4439; C07D 409/04; A61K 31/4184; C07D 403/04; C07D 235/04; C07D 417/12; A61K 31/427; C07D 409/14; A61K 31/422; C07D 413/14; A61P 25/28; A61P 25/24; A61P 25/06; A61P 3/10

(52) U.S. Cl. 514/338; 548/304.7; 514/394; 548/195; 514/371; 546/273.4; 514/378; 548/249

(57) ABSTRACT

Benzoimidazole Derivatives are provided. The compounds of the present invention are useful for Glycogen Synthase Kinase-3 Beta Inhibitors.

### PRIORITY

The present application claims the benefit of U.S. Provisional Application No. 61/084,770, filed on Jul. 30, 2008, the entire contents of which are incorporated by reference herein.

### TECHNICAL FIELD

The present invention relates to a compound for inhibiting glycogen synthase kinase-3 (GSK3) activity, a method for the preparation thereof, and a pharmaceutical composition containing the compound as an active ingredient.

### BACKGROUND ART

Glycogen synthase kinase-3 (GSK3) is a proline-directed serine-threonine kinase that was initially identified as a protein which inactivates glycogen synthase through phosphorylation. Two isoforms have been identified, alpha (GSK3alpha) and beta (GSK3beta), which show a high degree of amino acid homology to each other. Previous studies have reported that the GSK3beta is involved in energy metabolism, neural cell development, and body pattern formation (Plyte S E, et al., Biochim. Biophys. Acta, 1114:147-162, 1992).

Neurodegenerative naturopathies, including Alzheimer disease, are characterized by abnormal hyperphosphorylation of the microtubule-associated protein tau at proline-directed serine/threonine phosphorylation sites (Lee V M, et al., Annu. Rev. Neurosci. 24: 1121-1159, 2001.). GSK3beta has been identified as a prime candidate mediating aberrant tau phosphorylation at disease-associated sites (Hanger D P, et al., Neurosci. Lett. 147: 58-62, 1992, Ishiguro K, et al., J. Biol. Chem. 267: 10897-10901, 1992, Mandelkow E M, et al., FEBS Lett. 314: 315-321, 1992. and Paudel H K, et al., J. Biol. Chem. 268: 23512-23518, 1993.). Hence, GSK3beta is a promising target for therapeutic intervention in neurodegenerative tauopathies including Alzheimer disease.

Lithium carbonate, lithium citrate and lithium chloride are commonly used for the treatment of various disorders like mania, depression and migraine, and also used as an “augmenting” agent to increase the benefits of other standard drugs used for unipolar depression. Lithium is a GSK3beta inhibitor, and therefore, GSK3beta inhibition is a promising target for the treatment of various such disorders.

There have been reports that the activity of GSK3 in obese diabetic mice is about twice as high as that in control (Eldar-Finkelman H, et al., Diabetes, 48:1662-1666, 1999), and the activity and expression of GSK3 in patients with type 2 diabetes is significantly higher relatively to that in normal persons (Nikoulina S E, et al., Diabetes, 49:263-271, 2000). Therefore, GSK3 inhibitors are available for treatment of type 2 diabetes by reducing the activity of glucose synthase.

Taken together, GSK3beta inhibitors can be used for a broad spectrum of diseases such as Alzheimer disease, mania, depression, migraine and type 2 diabetes and there is a strong need to develop such inhibitors for the treatment and/or prevention of GSK3beta dependent diseases.

The present inventors have found that benzoimidazole derivatives can selectively inhibit the activity of GSK3beta and are therefore useful for treatment and/or prevention of GSK3beta dependent diseases.

### SUMMARY OF INVENTION

Accordingly, it is an object of the present invention to provide GSK3beta inhibitors having high inhibitory activity against GSK3beta.

It is another object of the present invention to provide a method for preparing such inhibitors.

It is a further object of the present invention to provide a pharmaceutical composition including said compounds, pharmaceutically acceptable salts, hydrates, solvates, and isomers thereof.

In accordance with one aspect of the present invention, there is provided a compound of formula (I), and a pharmaceutically acceptable salt, hydrate, solvate, or isomer thereof:

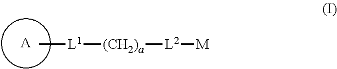

wherein,
ring A is (II), (III), (IV) (V), or (VI)

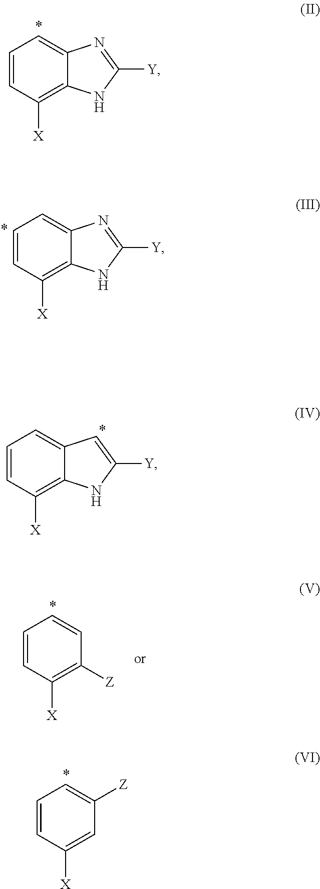

wherein

X is halogen or hydroxyl;

Y is hydrogen, phenyl, thiophen-2-yl, furan-2-yl, cyclopropyl, or cyclopentyl;

Z is a 5-10 membered heterocycle substituted carbonylamino; and
Ring A is substituted by -L¹-(CH₂)ₐ-L²-M at position *;
L¹ is —CONH—, —NHCO—, or a single bond;

L² is selected from the group consisting of —NH—, —O—, —CH(COOR¹)—, —CH(CH₂OH)—, —CH═CH— and a single bond, wherein R¹ is hydrogen or C₁-C₆ alkyl;

M is selected from the group consisting of hydroxyl, carboxyl, amide, C₁-C₆ alkyl, C₁-C₆ alkylcarbonyl, C₆-C₁₄ aryl, C₆-C₁₄ aryl C₁-C₆ alkyl, C₆-C₁₄ arylcarbonyl, C₆-C₁₄ arylsulfonyl, 5-14 membered saturated, unsaturated or aromatic heterocyclic group, 5-14 membered unsaturated or aromatic heterocyclic group substituted C₁-C₆ alkyl, 5-14 membered unsaturated or aromatic heterocyclic group substituted sulfonyl or —NR²R³;
wherein R² and R³ are independently C₁-C₆ alkyl;
the C₁-C₆alkyl, C₁-C₆ alkylcarbonyl, C₆-C₁₄ aryl, C₆-C₁₄ aryl C₁-C₆ alkyl, C₆-C₁₄ arylcarbonyl, C₆-C₁₄ arylsulfonyl, 5-14 membered unsaturated or aromatic heterocyclic group, 5-14 membered unsaturated or aromatic heterocyclic group substituted C₁-C₆alkyl, and 5-14 membered unsaturated or aromatic heterocyclic group substituted sulfonyl are optionally substituted by 1-3 substituent(s) each independently selected from group A;

wherein group A consists of hydroxyl, oxo, nitro, amino, amide, halogen, sulfamoyl, trifluolomethyl, p-toluenesulfonylamino, C₁-C₆ alkyl, C₁-C₆ alkoxy, C₁-C₆ alkylcarbonylamino, and C₁-C₆ alkylsulfonylamino; and

a is 0-5 integer.

### DESCRIPTION OF EMBODIMENTS

### Definitions

In this invention, “alkyl” refers to a straight chain or a branched chain hydrocarbon group which does not contain any hetero atoms or unsaturated carbon-carbon bonds. “C₁-C₆ alkyl” refers to an alkyl group which has 1-6 carbon atoms. “C₁-C₄alkyl” refers to an alkyl group which has 1-4 carbon atoms.

Examples of “C₁-C₆ alkyl” include, but are not limited to, methyl, ethyl, 1-propyl, 2-propyl, 2-methyl-1-propyl, 2-methyl-2-propyl, 1-butyl, 2-butyl, 1-pentyl, 2-pentyl, 3-pentyl, 2-methyl-1-butyl, 3-methyl-1-butyl, 2-methyl-2-butyl, 3-methyl-2-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2-methyl-3-pentyl, 3-methyl-3-pentyl, 2,3-dimethyl-1-butyl, 3,3-dimethyl-1-butyl, 2,2-dimethyl-1-butyl, 2-ethyl-1-butyl, 3,3-dimethyl-2-butyl, and 2,3-dimethyl-2-butyl.

In the present invention, “alkoxy” refers to a group represented by —OR, wherein R is alkyl.

“C₁-C₆ alkoxy” refers to an alkoxy group which has 1-6 carbon atoms. “C₁-C₄ alkoxy” refers to an alkoxy group which has 1-4 carbon atoms.

Examples of “C₁-C₆ alkoxy” include, but are not limited to, methoxy, ethoxy, 1-propyloxy, 2-propyloxy, 2-methyl-1-propyloxy, 2-methyl-2-propyloxy, and 1-butyloxy, and 2-butyloxy.

In the present invention, “carbonyl” refers to a group represented by —(C═O)—.

In this invention, “C₁-C₆ alkylcarbonyl” refers to a carbonyl group bound to the C₁-C₆ alkyl. “C₁-C₄ alkylcarbonyl” refers to a carbonyl group bound to the C₁-C₄ alkyl.

Examples of “C₁-C₆ alkylcarbonyl” include, but are not limited to, methylcarbonyl, ethylcarbonyl, 1-propylcarbonyl, 2-propylcarbonyl, n-butylcarbonyl, s-butylcarbonyl, t-butylcarbonyl, and 2-ethylbutylcarbyl.

In this invention, “amino” refers to a group represented by —NH₂ in which the hydrogens are optionally replaced by a substituent.

In the present invention, “C₁-C₆ alkylcarbonylamino” refers to an amino group bound to the C₁-C₆ alkylcarbonyl. “C₁-C₄ alkylcarbonylamino” refers to an amino group bound to the C₁-C₄ alkylcarbonyl.

Examples of “C₁-C₆ alkylcarbonylamino” include, but are not limited to, methylcarbonylamino, ethylcarbonylamino, 1-propylcarbonylamino, 2-propylcarbonylamino, n-butylcarbonylamino, s-butylcarbonylamino, t-butylcarbonylamino, and 2-ethylbutylcarbonylamino.

In this invention, “sulfonyl” is a group represented by —SO₂—.

In this invention, “C₁-C₆ alkylsulfonyl” refers to a sulfonyl group bound to the C₁-C₆ alkyl. “C₁-C₄ alkylsulfonyl” refers to a sulfonyl group bound to the C₁-C₄ alkyl.

Examples of “C₁-C₆ alkylsulfonyl” include, but are not limited to, methylsulfonyl, ethylsulfonyl, 1-propylsulfonyl, 2-propylsulfonyl, n-butylsulfonyl, s-butylsulfonyl, t-butylsulfonyl, and 2-ethylbutylsulfonyl.

In the present invention, “C₁-C₆ alkylsulfonylamino” refers to an amino group bound to the “C₁-C₆ alkylsulfonyl”. “C₁-C₄ alkylsulfonylamino” refers to an amino group bound to the “C₁-C₄ alkylsulfonyl”.

Examples of “C₁-C₆ alkylsulfonylamino” include, but are not limited to, methylsulfonylamino, ethylsulfonylamino, 1-propylsulfonylamino, 2-propylsulfonylamino, n-butylsulfonylamino, s-butylsulfonylamino, t-butylsulfonylamino, and 2-ethylbutylsulfonylamino.

In the present invention, “aryl” refers to an aromatic carbon ring system. “C₆-C₁₄ aryl” refers to a 6-14 membered aryl ring. “C₆-C₁₀aryl” refers to a 6-10 membered aryl ring.

Examples of “C₆-C₁₄ aryl” include, but are not limited to, phenyl, naphthyl, and anthryl.

In the invention, “C₆-C₁₄ aryl C₁-C₆ alkyl” refers to the “C₁-C₆ alkyl” in which a hydrogen atom is substituted by the “C₆-C₁₄ aryl”. “C₆-C₁₀arylC₁-C₄alkyl” refers to the “C₁-C₄ alkyl” in which a hydrogen atom is substituted by the “C₆-C₁₀ aryl”.

Examples of “C₆-C₁₄arylC₁-C₆alkyl” include, but are not limited to, benzyl, phenethyl, and anthrylmethyl.

In the present invention, “C₆-C₁₄ arylcarbonyl” refers to a carbonyl group bound to the “C₆-C₁₄ aryl”. “C₆-C₁₀ arylcarbonyl” refers to a carbonyl group bound to the “C₆-C₁₀ aryl”.

Examples of “C₆-C₁₄ arylcarbonyl” include, but are not limited to, phenylcarbonyl, naphthylcarbonyl, and anthrylcarbonyl.

In this invention, “C₆-C₁₄ arylsulfonyl” refers to a sulfonyl group bound to the “C₆-C₁₄ aryl”. “C₆-C₁₀ arylsulfonyl” refers to a sulfonyl group bound to the “C₆-C₁₀ aryl”.

Examples of “C₆-C₁₄arylsulfonyl” include, but are not limited to, phenylsulfonyl, naphthylsulfonyl, and anthrylsulfonyl.

In the present invention, “an unsaturated or aromatic heterocyclic group” refers to an unsaturated or aromatic heterocyclic group having one or more hetero atom in the ring system. “5-14 membered unsaturated or aromatic heterocyclic group” refers to an unsaturated or aromatic heterocyclic group in which the ring consists of 5-14 atoms. “5-10 membered unsaturated or aromatic heterocyclic group” refers to a unsaturated or aromatic heterocyclic group in which the ring consists of 5-10 atoms.

Examples of “5-14 membered unsaturated or aromatic heterocyclic group” include, but are not limited to, imidazolyl, pyrrolyl, pyridyl, thienyl, furyl, thiazolyl, pyrazolyl, pyrazolinyl, oxazolyl, isoxazolyl and indolyl.

In this invention, “5-14 membered unsaturated or aromatic heterocyclic group substituted C₁-C₆ alkyl” refers to the “C₁-C₆ alkyl” in which a hydrogen atom is substituted by the “5-14 membered unsaturated or aromatic heterocyclic group”. “5-10 membered unsaturated or aromatic heterocyclic group substituted C₁-C₄ alkyl” refers to the “C₁-C₄ alkyl” in which a hydrogen atom is substituted by the “5-10 membered unsaturated or aromatic heterocyclic group”.

Examples of “5-14 membered unsaturated or aromatic heterocyclic group substituted C₁-C₆ alkyl” include, but are not limited to, imidazolylmethyl, pyrrolylmethyl, pyridylmethyl, thienylmethyl, furylmethyl, thiazolylmethyl, pyrazolylmethyl, pyrazolinylmethyl, oxazolylmethyl, isoxazolylmethyl, and indolylmethyl.

In this invention, “5-14 membered unsaturated or aromatic heterocyclic group substituted sulfonyl” refers to a sulfonyl group bound to the 5-14 membered unsaturated or aromatic heterocyclic group”. “5-10 membered unsaturated or aromatic heterocyclic group substituted sulfonyl” refers to a sulfonyl group bound to “5-10 membered unsaturated or aromatic heterocyclic group”.

Examples of “5-14 membered unsaturated or aromatic heterocyclic group substituted sulfonyl” include, but are not limited to, imidazolylsulfonyl, pyrrolylsulfonyl, pyridylsulfonyl, thienylsulfonyl, furylsulfonyl, thiazolylsulfonyl, pyrazolylsulfonyl, pyrazolinylsulfonyl, oxazolylsulfonyl, isoxazolylsulfonyl, and indolylsulfonyl.

In this invention, “5-10 membered unsaturated or aromatic heterocyclic group substituted carbonylamino” refers to an amino group bound to a carbonyl group bound to the “5-10 membered unsaturated or aromatic heterocyclic group”.

Examples of “5-10 membered unsaturated or aromatic heterocyclic group substituted carbonylamino” include, but are not limited to, imidazolylcarbonylamino, pyrrolylcarbonylamino, pyridylcarbonylamino, thienylcarbonylamino, furylcarbonylamino, thiazolylcarbonylamino, pyrazolylcarbonylamino, pyrazolinylcarbonylamino, oxazolylcarbonylamino, isoxazolylcarbonylamino, and indolylcarbonylamino.

In the present invention, “a saturated heterocyclic group” refers to a saturated heterocyclic group having one or more hetero atom in the ring system. “5-14 membered saturated heterocyclic group” refers to a saturated heterocyclic group in which the ring consists of 5-14 atoms. “5-10 membered saturated heterocyclic group” refers to a saturated heterocyclic group in which the ring consists of 5-10 atoms.

Examples of “5-14 membered saturated heterocyclic group” include, but are not limited to, pyrrolidinyl, piperidinyl, piperazinyl, morpholinyl and thiomorpholinyl.

A salt is defined as the product formed from the neutralisation reaction of acids and bases. Salts are ionic compounds composed of cations (positively charged ions) and anions (negative ions) so that the product is electrically neutral. These component ions can be inorganic as well as organic.

Hydrate is a term used in inorganic chemistry and organic chemistry to indicate that a substance contains water. Solvate refers to a molecule in a solution complexed by solvent molecules. Isomers are compounds with the same molecular formula but different structural formulae. More specifically, isomer includes geometric isomer, optical isomer, stereoisomer, tautomer of the compound, and mixtures thereof.

The present invention provides a compound represented by formula (I):

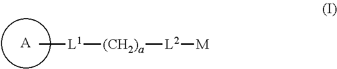

Among the compounds of formula (I) of the present invention, the preferred are those wherein:
A is (II),

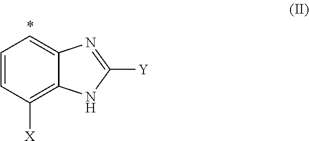

wherein
X is halogen or hydroxyl;
Y is phenyl, thiophen-2-yl, furan-2-yl, cyclopropyl, or cyclopentyl;
   Ring (II) is substituted by -L¹-(CH₂)ₐ-L²-M at position *;

### L¹ is —CONH— or —NHCO—;

L² is selected from the group consisting of —NH—, —O—, —CH(COOR¹)—, —CH(CH₂OH)—, and a single bond, wherein R¹ is hydrogen or C₁-C₆ alkyl;

M is selected from the group consisting of hydroxyl, carboxyl, amide, C₁-C₆ alkyl, C₁-C₆ alkylcarbonyl, C₆-C₁₄ aryl, C₆-C₁₄ aryl C₁-C₆ alkyl, C₆-C₁₄ arylcarbonyl, C₆-C₁₄ arylsulfonyl, 5-14 membered saturated, unsaturated or aromatic heterocyclic group, 5-14 membered unsaturated or aromatic heterocyclic group substituted C₁-C₆alkyl, 5-14 membered unsaturated or aromatic heterocyclic group substituted sulfonyl or —NR²R³;
wherein R² and R³ are independently C₁-C₆ alkyl;
   the C₁-C₆ alkyl, C₁-C₆alkylcarbonyl, C₆-C₁₄ aryl, C₆-C₁₄ aryl C₁-C₆ alkyl, C₆-C₁₄ arylcarbonyl, C₆-C₁₄ arylsulfonyl, 5-14 membered unsaturated or aromatic heterocyclic group, 5-14 membered unsaturated or aromatic heterocyclic group substituted by C₁-C₆ alkyl, and 5-14 membered unsaturated or aromatic heterocyclic group substituted sulfonyl are optionally substituted by 1-3 substituent(s) each independently selected from group A;
wherein group A consists of hydroxyl, oxo, nitro, amino, amide, halogen, sulfamoyl, trifluolomethyl, p-toluenesulfonylamino, C₁-C₆ alkyl, C₁-C₆ alkoxy, C₁-C₆ alkylcarbonylamino, and C₁-C₆ alkylsulfonylamino; and
   a is an integer from 0-5.

In a preferred embodiment, the present invention provides compounds represented by following formula (I-II) or a salt, hydrate, solvate, or isomer thereof:

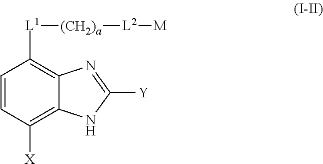

wherein
L′ is —CONH—;
L² is a single bond;
M is C₆-C₁₀ aryl or 5-10 membered unsaturated or aromatic heterocyclic group having 1-2 hetero atom(s) selected from the group consisting of N, O, and S, which are optionally substituted by 1 or 2 substituent(s) each independently selected from the group A; and
X, Y, and a are defined as in above embodiment represented by formula (I).

In this embodiment, M is selected from the group consisting of phenyl, imidazole-1-yl, imdazole-2-yl, imidazole-5-yl, thiophen-2-yl, pyrole-2-yl, 1,3-thiazole-2-yl, 2-pyrazoline-4-yl, and isoxazole-4-yl, which are optionally substituted by 1-2 substituent(s) each independently selected from following group B, and Y is selected from the group consisting of thiophen-2-yl, furan-2-yl, phenyl, cyclopropyl, and cyclopentyl.

Group B consists of fluoro, hydroxyl, oxo, amino, methyl, methoxy, and sulfamoyl.

Preferred compounds include those selected from the group consisting of: Example Nos. 8, 9, 10, 20, 21, 22, 23, 35, 37, 44, 45, 57, 62, 76, 77, 78, 79, 80, 84, 85, 86, 90, 91, 92, 93, 94, 95, 96, 101 and 102 listed in Table 1 below; and the pharmaceutically acceptable salts, prodrugs, hydrates and solvates of the forgoing compounds.

TABLE 1

| Example | | |
|---|---|---|
| No. | structure | compound |
| 8 | 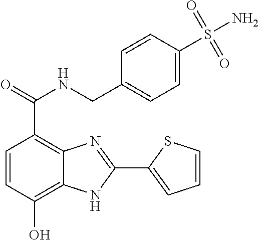 | 7-Hydroxy-N-(4- sulfamoylbenzyl)- 2-(thiophen-2-yl)- 1H-benzo [d]imidazole-4- carboxamide |
| 9 | 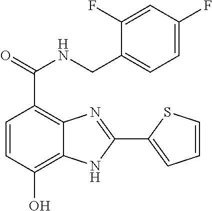 | N-(2,4- Difluorobenzyl)-7- hydroxy-2- (thiophen-2-yl)- 1H-benzo [d]imidazole- 4-carboxamide |
| 10 | 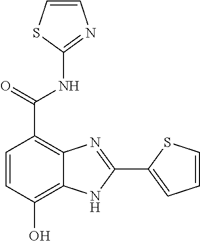 | 7-Hydroxy-N- (thiazol-2-yl)-2- (thiophen-2-yl)- 1H-benzo[d] imidazole-4- carboxamide |
| 20 | 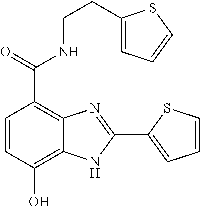 | 7-Hydroxy-2- (thiophen-2-yl)- N-[2-(thiophen- 2-yl)ethyl]-1H- benzo[d] imidazole-4- carboxamide |
| 21 | 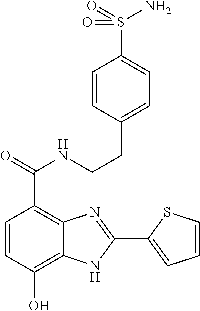 | 7-Hydroxy-N-(4- sulfamoyl- phenethyl)- 2-(thiophen-2- yl)-1H- benzo[d] imidazole-4- carboxamide |
| 22 | 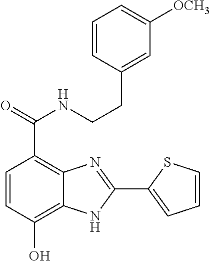 | 7-Hydroxy-N-(3- methoxyphenethyl)- 2-(thiophen-2- yl)-1H- benzo[d] imidazole-4- carboxamide |
| 23 | 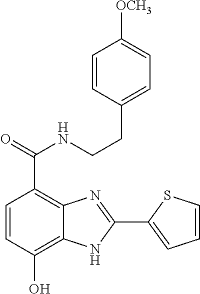 | 7-hydroxy-N-(3- methoxyphenethyl)- 2-(thiophen- 2-yl)-1H- benzo[d] imidazole-4- carboxamide |
| 35 | 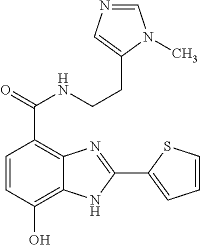 | 7-Hydroxy-N- [2-(1-methyl- 1H-imidazol-5- yl)ethyl]-2- (thiophen-2-yl)- 1H-benzo[d] imidazole-4- carboxamide |
| 37 | 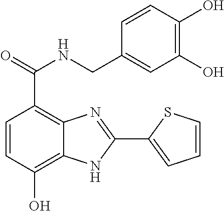 | N-(3,4- Dihydroxybenzyl)- 7-hydroxy-2- (thiophen- 2-yl)-1H- benzo[d] imidazole-4- carboxamide |
| 44 | 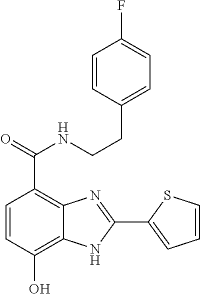 | N-(4- Fluorophenethyl)- 7-hydroxy-2- (thiophen-2-yl)- 1H-benzo [d]imidazole- 4-carboxamide |
| 45 | 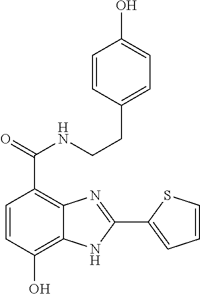 | 7-Hydroxy-N-(4- hydroxyphenethyl)- 2-(thiophen- 2-yl)-1H- benzo[d] imidazole-4- carboxamide |
| 57 | 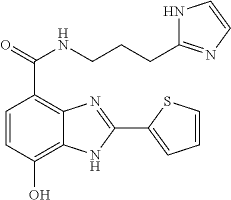 | N-3-(1H-Imidazol- 2-yl)propyl)-]7- hydroxy-2- (thiophen-2-yl)- 1H-benzo[d] imidazole-4- carboxamide |
| 62 | 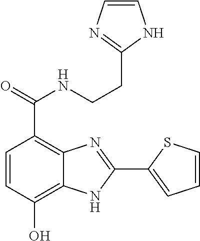 | N-[2-(1H-Imidazol- 2-yl)ethyl]-7- hydroxy-2- (thiophen-2- yl)-1H- benzo[d] imidazole-4- carboxamide |
| 76 | 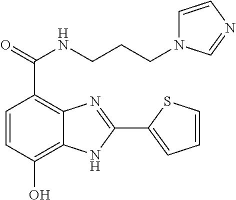 | N-[3-(1H- Imidazol-1-yl) propyl]-7- hydroxy-2- (thiophen-2-yl)- 1H-benzo[d] imidazole-4- carboxamide |
| 90 | 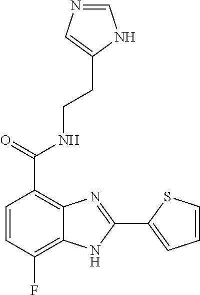 | N-[2-(1H- Imidazol-5-yl) ethyl]-7-fluoro- 2-(thiophen-2-yl)- 1H-benzo[d] imidazole-4- carboxamide |
| 77 | 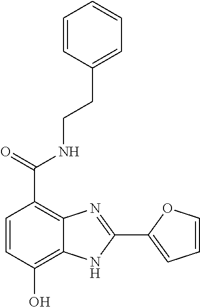 | 2-(Furan-2-yl)- 7-hydroxy-N- phenethyl-1H- benzo[d]imidazole- 4-carboxamide |
| 78 | 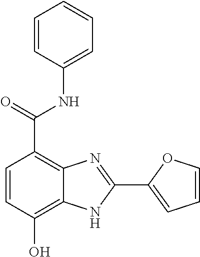 | 2-(Furan-2-yl)- 7-hydroxy-N- phenyl-1H- benzo[d] imidazole-4- carboxamide |
| 79 | 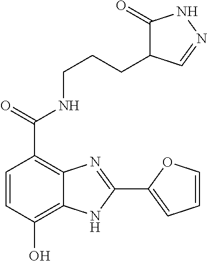 | 7-Hydroxy-N- [3-(5-oxo-4,5- dihydro-1H- pyrazol-4-yl) propyl]- 2-(thiophen- 2-yl)-1H-benzo [d]imidazole-4- carboxamide |
| 80 | 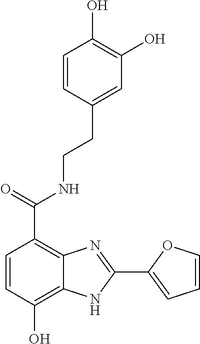 | N-(3,4- Dihydroxy- phenethyl)- 2-(furan-2-yl)- 7-hydroxy-1H- benzo[d] imidazole-4- carboxamide |
| 84 | 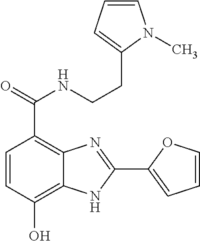 | 2-(Furan-2-yl)-7- hydroxy-N-[2- (1-methyl-1H- pyrrol-2-yl) ethyl]-1H-benzo [d]imidazole-4- carboxamide |
| 85 | 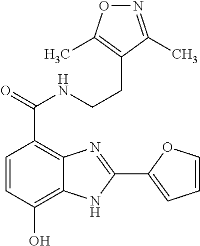 | N-(2-(3,5- dimethylisoxazol- 4-yl)ethyl)-2- (furan-2-yl)-7- hydroxy-1H-benzo [d]imidazole-4- carboxamide |
| 86 | 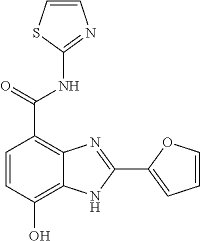 | 2-(Furan-2-yl)- 7-hydroxy-N- (thiazol-2-yl)- 1H-benzo[d] imidazole-4- carboxamide |
| 92 | 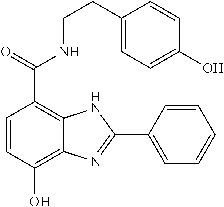 | 4-Hydroxy-N-(4- hydroxyphenethyl)- 2-phenyl- 1H-benzo[d] imidazole-7- carboxamide |
| 93 | 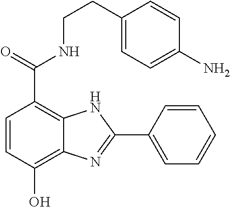 | N-(4- Aminophenethyl)- 4-hydroxy-2- phenyl-1H- benzo[d] imidazole-7- carboxamide |
| 94 | 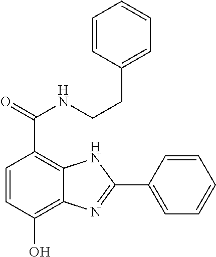 | 4-Hydroxy-N- phenethyl-2- phenyl-1H-benzo [d]imidazole-7- carboxamide |
| 91 | 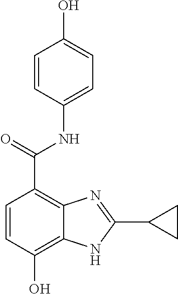 | 2-Cyclopropyl-N- (4-hydroxyphenyl)- 4-methoxy- 1H-benzo[d] imidazole-7- carboxamide |
| 101 | 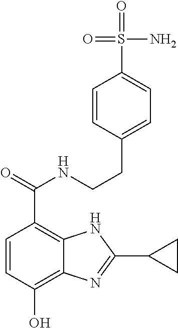 | 2-Cyclopropyl-4- hydroxy-N-(4- sulfamoyl- phenethyl)- 1H-benzo [d]imidazole-7- carboxamide |
| 102 | 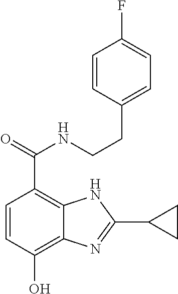 | 2-Cyclopropyl- N-(4- fluorophenethyl)- 4-hydroxy-1H- benzo[d] imidazole-7- carboxamide |
| 95 | 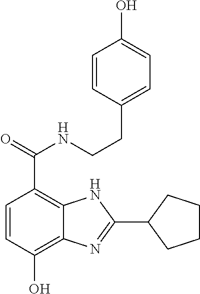 | 2-Cyclopentyl-4- hydroxy-N-(4- hydroxyphenethyl)- 1H-benzo [d]imidazole-7- carboxamide |
| 96 | 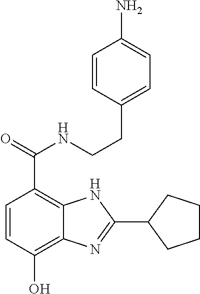 | N-(4- Aminophenethyl)- 2-cyclopentyl- 4-hydroxy-1H- benzo[d] imidazole-7- carboxamide |

In another preferred embodiment, the present invention provides a compound represented by following formula (I-II) or a salt, hydrate, solvate, or isomer thereof:

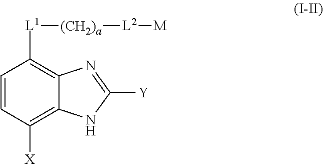

wherein
L¹ is —CONH—;
L² is —NH—;
M is C₁-C₄ alkyl, C₁-C₄ alkylcarbonyl, C₆-C₁₀ arylcarbonyl, C₆-C₁₀ arylsulfonyl, 5-10 membered unsaturated or aromatic heterocyclic group having 1-2 hetero atom(s) selected from the group consisting of N, O, and S or sulfonyl substituted by 5-10 membered unsaturated or aromatic heterocyclic group having 1-2 hetero atom(s) selected from the group consisting of N, O, and S, which are optionally substituted by 1 or 2 substituent(s) each independently selected form the group A; and
   X, Y, and a are defined as in the above embodiment represented by formula (I).

In this embodiment, M is selected from the group consisting of ethyl, isopropyl, methylcarbonyl, pyridine-2-yl, phenylcarbonyl, phenylsulfonyl, and 4-pyridilsulfonyl, which are optionally substituted by 1-2 substituent(s) each independently selected from following group C, and Y is selected from the group consisting of thiophen-2-yl and furan-2-yl.

Group C consists of chloro, hydroxyl, methyl, methylcarbonylamino, methylsulfonylamino, and p-toluenesulfonylamino.

In one preferred embodiment, the present invention provides the compound selected from the group consisting of: Example Nos. 11, 12, 38, 39, 40, 41, 42, 43, 69, 70 and 89 listed in Table 2 below, and the pharmaceutically acceptable salts, prodrugs, hydrates and solvates of the forgoing compounds.

TABLE 2

| Example | | |
|---|---|---|
| No. | structure | Compound |
| 11 | 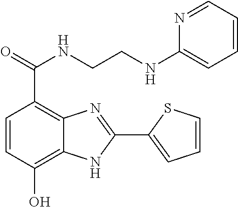 | 7-Hydroxy-N-[2-(pyridin-2-ylamino)ethyl]-2-(thiophen-2- yl)-1H-benzo[d]imidazole-4-carboxamide |
| 12 | 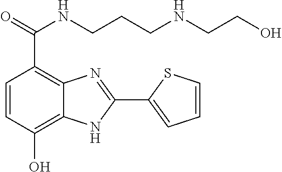 | 7-Hydroxy-N-[3-(2-hydroxyethylamino)propyl]-2- (thiophen-2-yl)-1H-benzo[d]imidazole-4-carboxamide |
| 38 | 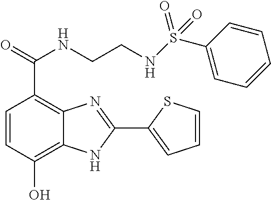 | 7-Hydroxy-N-[2-(phenylsulfonamido)ethyl]-2-(thiophen- 2-yl)-1H-benzo[d]imidazole-4-carboxamide |
| 39 | 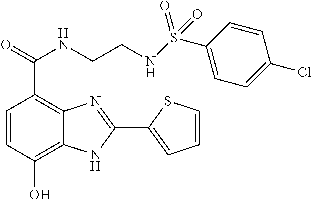 | N-[2-(4-Chlorophenylsulfonamido)ethyl]-7-hydroxy-2- (thiophen-2-yl)-1H-benzo[d]imidazole-4-carboxamide |
| 40 | 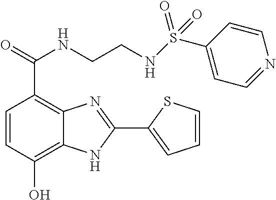 | 7-Hydroxy-N-[2-(pyridine-4-sulfonamido)ethyl]-2- (thiophen-2-yl)-1H-benzo[d]imidazole-4-carboxamide |
| 41 | 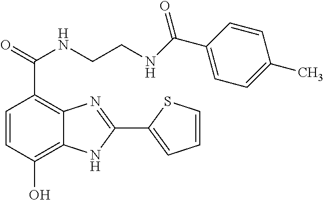 | 7-Hydroxy-N-[2-(4-methylbenzamido)ethyl]-2-(thiophen- 2-yl)-1H-benzo[d]imidazole-4-carboxamide |
| 42 | 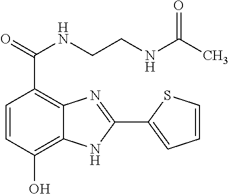 | N-(2-Acetamidoethyl)-7-hydroxy-2-(thiophen-2-yl)-1H- benzo[d]imidazole-4-carboxamide |
| 43 | 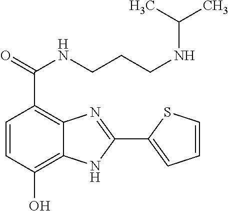 | N-[3-(Isopropylamino)propyl]-7-hydroxy-2-(thiophen-2- yl)-1H-benzo[d]imidazole-4-carboxamide |
| 69 | 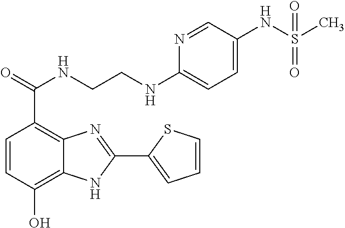 | 7-Hydroxy-N-{2-[5-(methylsulfonamido)pyridin-2- ylamino]ethyl}-2-(thiophen-2-yl)-1H-benzo[d]imidazole-4- carboxamide |
| 70 | 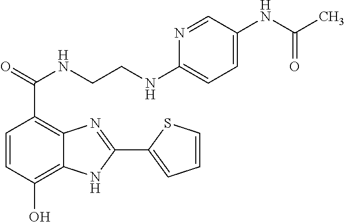 | N-[2-(5-Acetamidopyridin-2-ylamino)ethyl]-7-hydroxy-2- (thiophen-2-yl)-1H-benzo[d]imidazole-4-carboxamide |
| 89 | 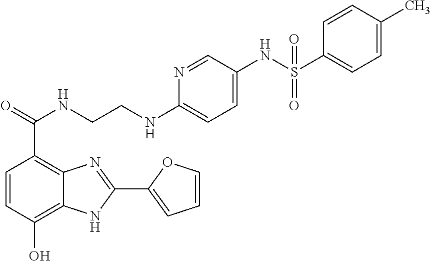 | 2-(Furan-2-yl)-7-hydroxy-N-{2-[5-(4- methylphenylsulfonamido)pyridin-2-ylamino]ethyl}- 1H-benzo[d]imidazole-4-carboxamide |

In another preferred embodiment, the present invention provides a compound represented by following formula (I-II) or a salt thereof:

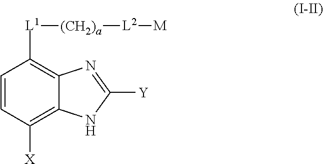

wherein
L¹ is —CONH—;
L² is —CH(COOR¹)—, wherein R¹ is hydrogen or C₁-C₄ alkyl;
M is C₁-C₄ alkyl, C₆-C₁₀ aryl C₁-C₄ alkyl or C₁-C₄ alkyl substituted by 5-10 membered unsaturated or aromatic heterocyclic group having 1-2 hetero atom(s) selected from the group consisting of N, O, and S, which are optionally substituted by 1 or 2 substituent(s) each independently selected from the group A; and
   X, Y, and a are defined as in the above embodiment represented by formula (I).

In this embodiment, M is selected from the group consisting of methyl, phenylmethyl, indole-3-ylmethyl, and imidazole-4-ylmethyl, which are optionally substituted by 1-2 hydroxyl, and Y is thiophen-2-yl.

In one preferred embodiment, the present invention provides the compound selected from the group consisting of: Example Nos. 13, 14, 15, 16, 71 and 72 listed in Table 3 below, and the pharmaceutically acceptable salts, prodrugs, hydrates and solvates of the forgoing compounds.

TABLE 3

| Example | | |
|---|---|---|
| No. | structure | compound |
| 13 | 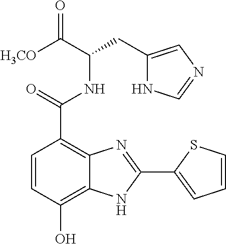 | (S)-Methyl 2-[7-Hydroxy-2- (thiophen-2-yl)-1H- benzo[d] imidazole-4- carboxamido]-3- (1H-imidazol- 5-yl)propanoate |
| 14 | 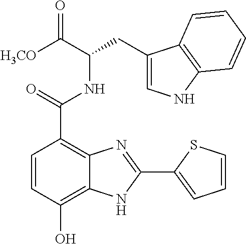 | (S)-Methyl 2-[7-Hydroxy-2- (thiophen-2-yl)- 1H-benzo[d] imidazole-4- carboxamido]-3- (1H-indol-3-yl) propanoate |
| 15 | 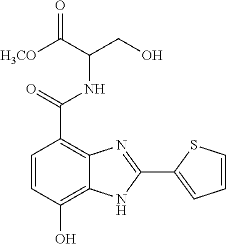 | Methyl 3-Hydroxy-2- [7-hydroxy-2- (thiophen-2-yl)- 1H-benzo[d] imidazole-4- carboxamido] propanoate |
| 16 | 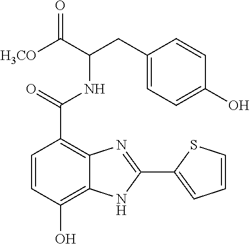 | Methyl 2-[7-Hydroxy-2- (thiophen-2-yl)- 1H-benzo[d] imidazole-4- carboxamido]-3- (4-hydroxyphenyl) propanoate |
| 71 | 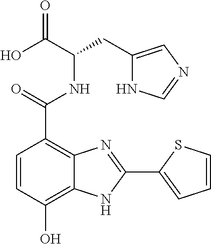 | (S)-2-[7-Hydroxy-2- (thiophen-2-yl)-1H- benzo[d]imidazole-4- carboxamido]-3- (1H-imidazol-5- yl)propanoic Acid |
| 72 | 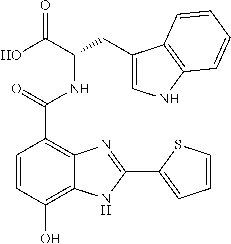 | (S)-2-[7-Hydroxy- 2-(thiophen-2-yl)- 1H-benzo[d] imidazole-4- carboxamido]-3- (1H-indol-3-yl) propanoic Acid |

In another preferred embodiment, the present invention provides a compound represented by following formula (I-II) or a salt, hydrate, solvate, or isomer thereof:

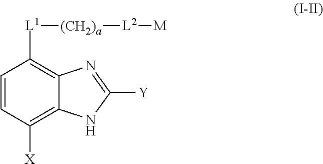

wherein
L¹ is —CONH—;
L² is —O—;
M is C₆-C₁₀ aryl or 5-10 membered unsaturated or aromatic heterocyclic group having 1-2 hetero atom(s) selected from the group consisting of N, O, and S, which are optionally substituted by 1 or 2 substituent(s) each independently selected from the group A;
   X, Y, and a are defined in the above embodiment represented by formula (I).

In this embodiment, M is phenyl or pyridine-2-yl, which is optionally substituted by 1 or 2 substituent(s) each independently selected from following group D, and Y preferably consists of thiophen-2-yl.

Group D consists of amide, nitro, trifluoromethyl, and p-toluenesulfonylamino.

In one preferred embodiment, the present invention provides the compound selected from the group consisting of: Example Nos. 49, 50, 73 and 74 listed in Table 4 below, and the pharmaceutically acceptable salts, prodrugs, hydrates and solvates of the forgoing compounds.

TABLE 4

| Example | | |
|---|---|---|
| No. | structure | compound |
| 49 | 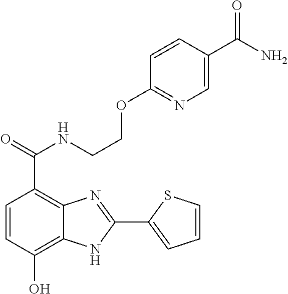 | N-[2-(5-Carbamoylpyridin-2- yloxy)ethyl]-7-hydroxy-2-(thiophen- 2-yl)-1H-benzo[d]imidazole-4-carboxamide |
| 50 | 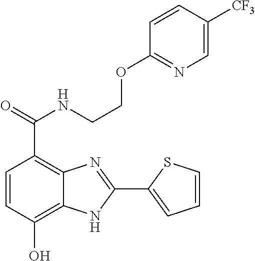 | 7-Hydroxy-2-(thiophen-2-yl)- N-[2-(5-(trifluoromethyl)pyridine-2- yloxy)ethyl]-1H-benzo[d] imidazole-4-carboxamide |
| 73 | 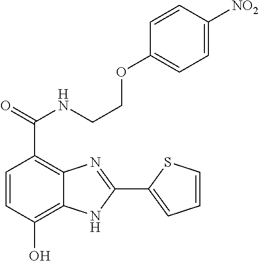 | 7-Hydroxy-N-[2-(4-nitrophenoxy) ethyl]-2-(thiophen-2-yl)-1H- benzo[d]imidazole-4-carboxamide |
| 74 | 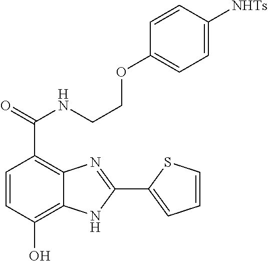 | 7-Hydroxy-N-{2-[4-(4- methylphenylsulfonamido)phenoxy]ethyl}- 2-(thiophen-2-yl)-1H-benzo [d]imidazole-4-carboxamide |

In another preferred embodiment, the present invention provides the compounds represented by following formula (I-II) or a salt, hydrate, solvate, or isomer thereof:

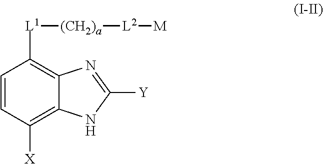

wherein
L¹ is —CONH—;
L² is —CH(CH₂OH)—;
M is selected from the group consisting of hydroxyl, C₁-C₄ alkyl, C₆-C₁₀ aryl C₁-C₄ alkyl, and C₁-C₄ alkyl substituted by 5-10 membered unsaturated or aromatic heterocyclic group having 1-2 hetero atom(s) selected from the group consisting of N, O, and S, the C₁-C₄ alkyl, C₆-C₁₀ aryl C₁-C₄ alkyl, and 5-10 membered unsaturated or aromatic heterocyclic group having 1-2 hetero atom(s) are optionally substituted by 1 or 2 substituent(s) each independently selected from the group A; and
   X, Y, and a are defined as in the above embodiment represented by formula (I).

In this embodiment, M is preferably hydroxyl, phenylmethyl, t-butyl, or imidazole-5-ylmethyl, and Y is selected from the group consisting of thiophen-2-yl and cyclopropyl.

In one preferred embodiment, the present invention provides the compound selected from the group consisting of: Example Nos. 17, 18, 19 and 97 listed in Table 5 below, and the pharmaceutically acceptable salts, prodrugs, hydrates and solvates of the forgoing compounds.

TABLE 5

| Example | | |
|---|---|---|
| No. | structure | compound |
| 17 | 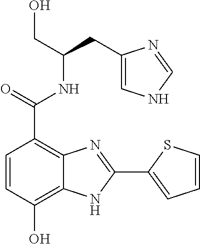 | (R)-7-Hydroxy-N-[1- hydroxy-3-(1H-imidazol- 4-yl)propan-2-yl]- 2-(thiophen-2-yl)-1H- benzo[d]imidazole-4- carboxamide |
| 18 | 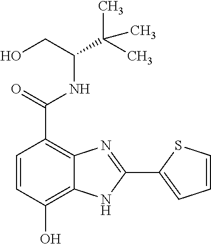 | (S)-7-Hydroxy-N-(1- hydroxy-3,3- dimethylbutan-2-yl)-2- (thiophen-2-yl)-1H- benzo[d]imidazole-4- carboxamide |
| 19 | 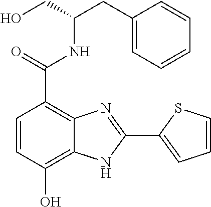 | (S)-7-Hydroxy-N-(1- hydroxy-3- phenylpropan-2-yl)- 2-(thiophen- 2-yl)-1H-benzo[d] imidazole-4- carboxamide |
| 97 | 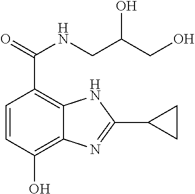 | 2-Cyclopropyl-N- (2,3-dihydroxypropyl)- 4-hydroxy-1H-benzo [d]imidazole-7- carboxamide |

In another preferred embodiment, the present invention provides a compound represented by following formula (I-II) or a salt, hydrate, solvate, or isomer thereof:

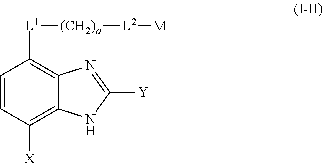

wherein
L¹ is —CONH—;
L² is a single bond;
M is —NR²R³;
   wherein R² and R³ are independently C₁-C₄ alkyl optionally substituted by 1 or 2 substituent(s) each independently selected from the group A; and
   X, Y, and a are defined in the above embodiment represented by formula (I).

In this embodiment, Y is selected from the group consisting of thiophen-2-yl and cyclopropyl.

In one preferred embodiment, the present invention provides the compound selected from the group consisting of: Example Nos. 36 and 98 listed in Table 6 below, and the pharmaceutically acceptable salts, prodrugs, hydrates and solvates of the forgoing compounds.

TABLE 6

| Example | | |
|---|---|---|
| No. | structure | compound |
| 36 | 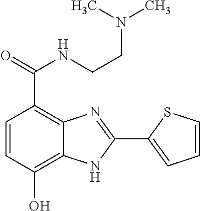 | N-[2-(dimethylamino)ethyl]- 7-hydroxy-2- (thiophen-2-yl)-1H- benzo[d]imidazole-4- carboxamide |
| 98 | 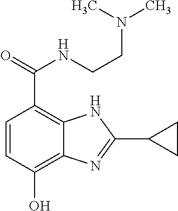 | 2-Cyclopropyl-N-(2- (dimethylamino)ethyl)-4- hydroxy-1H-benzo [d]imidazole-7-carboxamide |

In another preferred embodiment, the present invention provides a compound represented by following formula (I-II) or a salt, hydrate, solvate, or isomer thereof:

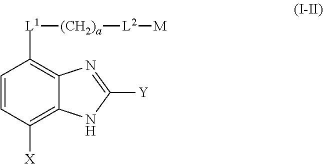

wherein
L¹ is —NHCO—;
L² is —NH—, —CH═CH— or a single bond;
M is C₆-C₁₀ aryl or 5-10 membered unsaturated or aromatic heterocyclic group having 1-2 hetero atom(s) selected from the group consisting of N, O, and S, which are optionally substituted by 1 or 2 substituent(s) each independently selected from the group A; and
   X, Y, and a are defined as in the above embodiment represented by formula (I).

In this embodiment, M is preferably phenyl optionally having 1 or 2 hydroxyl, or imidazol-5-yl and Y is cyclopropyl or thiophen-2-yl.

In one preferred embodiment, the present invention provides the compound selected from the group consisting of: Example Nos. 107, 108, 120 and 121 listed in Table 7 below, and the pharmaceutically acceptable salts, prodrugs, hydrates and solvates of the forgoing compounds.

TABLE 7

| Example | | |
|---|---|---|
| No. | structure | compound |
| 107 | 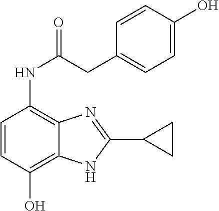 | N-(2-cyclopropyl-7-hydroxy-1H-benzo[d]imidazol-4-yl)-2-(4- hydroxyphenyl)acetamide |
| 108 | 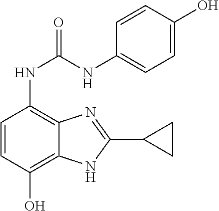 | 1-(2-Cyclopropyl-7-hydroxy-1H-benzo[d]imidazol-4-yl)-3-(4- hydroxyphenyl)urea |
| 120 | 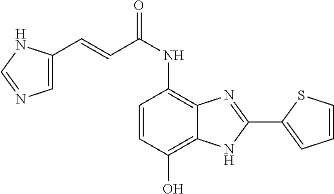 | (E)-3-(1H-imidazol-5-yl)-N-(7-methoxy-2-(thiophen-2-yl)-1H-benzo [d]imidazol-4-yl)acrylamide |
| 121 | 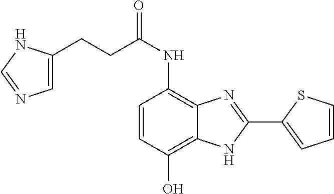 | N-(7-hydroxy-2-(thiophen-2-yl)-1H-benzo[d]imidazol-4-yl)-3-(1H- imidazol-5-yl)propanamide |

In another preferred embodiment, the present invention provides a compound represented by following formula (I-III) or a salt, hydrate, solvate, or isomer thereof:

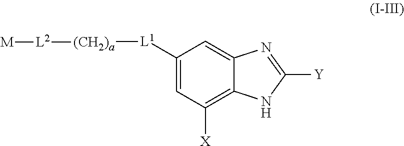

wherein
L¹ is —CONH— or a single bond;
L² is a single bond;
M is amide or 5-10 membered unsaturated or aromatic heterocyclic group having 1 or 2 hetero atom(s) selected from the group consisting of N, O, and S optionally substituted by 1 or 2 substituent(s) each independently selected from the group A; and
   X, Y, and a are defined in the above embodiment represented by formula (I).

In this embodiment, Y is thiophen-2-yl.

In one preferred embodiment, the present invention provides the compound selected from the group consisting of: Example Nos. 65 and 66 listed in Table 8 below, and the pharmaceutically acceptable salts, prodrugs, hydrates and solvates of the forgoing compounds.

TABLE 8

| Example | | |
|---|---|---|
| No. | structure | compound |
| 65 | 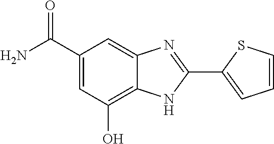 | 7-Hydroxy-2-(thiophen-2-yl)-1H-benzo[d]imidazole-5- carboxamide |
| 66 | 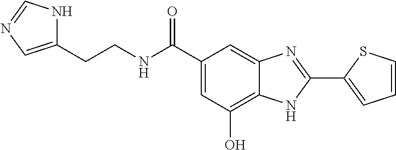 | N-[2-(1H-Imidazol-5-yl)ethyl]-7-hydroxy-2-(thiophen-2-yl)- 1H-benzo[d]imidazole-5-carboxamide |

In another preferred embodiment, the present invention provides a compound represented by following formula (I-IV) or a salt, hydrate, solvate, or isomer thereof:

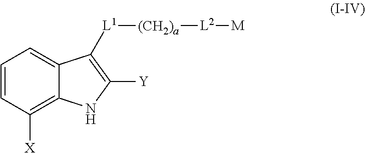

wherein
L¹ is —CONH—;
L² is a single bond;
M is 5-10 membered unsaturated or aromatic heterocyclic group having 1 or 2 hetero atom(s) selected from the group consisting of N, O, and S optionally substituted by 1 or 2 substituent(s) each independently selected from the group A; and
   X, Y, and a are defined as in the embodiment represented by formula (I).

In this embodiment, Y is hydrogen.

In one preferred embodiment, the present invention provides the compound of Example No. 110 listed in Table 9 below, and the pharmaceutically acceptable salts, prodrugs, hydrates and solvates of the forgoing compound.

TABLE 9

| Example | | |
|---|---|---|
| No. | structure | compound |
| 110 | 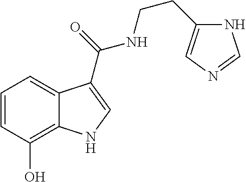 | N-[2-(1H-Imidazol- 5-yl)ethyl]-7- hydroxy-1H-indole-3- carboxamide |

In another preferred embodiment, the present invention provides compounds represented by following formula (I-V), (I-VI) or a salt, hydrate, solvate, or isomer thereof:

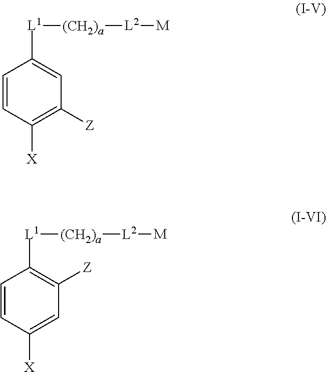

wherein
L¹ is —CONH—;
L² is a single bond;
M is 5-10 membered saturated, unsaturated or aromatic heterocyclic group having 1 or 2 hetero atom(s) selected from the group consisting of N, O, and S optionally substituted by 1 or 2 substituent(s) each independently selected from the group A; and
   X, Z, and a are defined as in the above embodiment represented by formula (I).

In this embodiment, Z is preferably thiophen-2-ylcarbonylamino.

In one preferred embodiment, the present invention provides the compound of Example Nos. 112 and 122 listed in Table 10 below, and the pharmaceutically acceptable salts, prodrugs, hydrates and solvates of the forgoing compound.

TABLE 10

| Example | | |
|---|---|---|
| No. | structure | compound |
| 112 | 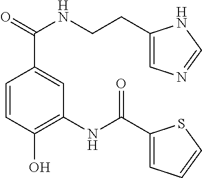 | N-{5-[2-(1H- Imidazol-5-yl) ethylcarbamoyl]- 2- hydroxyphenyl} thiophene-2- carboxamide |
| 122 | 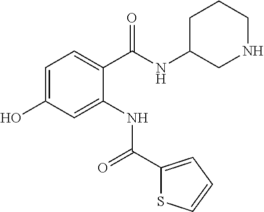 | N-(5-hydroxy- 2-(piperidin-3- ylcarbamoyl) phenyl)thiophene- 2-carboxamide |

In another preferred embodiment, the present invention provides a compound represented by formula (I-VI) or a salt, hydrate, solvate, or isomer thereof:

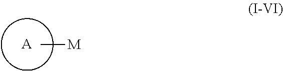

wherein
Ring A is represented by the formula below;

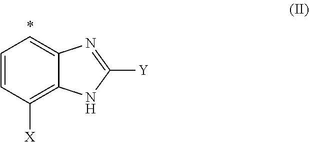

M is carboxyl;
   X, Y, Z and a are defined as in the above embodiment represented by formula (I).

In this embodiment, ring A is preferably the formula (II).

In one preferred embodiment, the present invention provides the compound of: Example No. 1 listed in Table 11 below, and the pharmaceutically acceptable salts, prodrugs, hydrates and solvates of the forgoing compounds.

TABLE 11

| Example | | |
|---|---|---|
| No. | Structure | Compound |
| 1 | 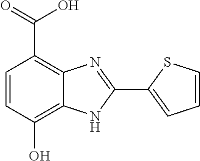 | 7-Hydroxy-2- (thiophen-2-yl)- 1H-benzo[d]imidazole-4- carboxylic acid |

The compound of formula (I) of the present invention may be in the form of a pharmaceutically acceptable salt derived from an inorganic or organic acid, and representative examples of the pharmaceutically acceptable salt derived from an inorganic or organic acid include salts obtained by adding an inorganic acid such as hydrochloric acid, hydrobromic acid, phosphoric acid or sulfonic acid, or organic carboxylic acids such as acetic acid, trifluoroacetic acid, citric acid, formic acid, maleic acid, oxalic acid, succinic acid, benzoic acid, tartaric acid, fumaric acid, mandelic acid, ascorbic acid or malic acid, methanesulfonic acid, or para toluenesulfonic acid, which do not limit its scope, to the compound of formula (I). Such acids may be prepared by the conventional processes, and other acids, which themselves are not pharmaceutically acceptable, including oxalic acid may be employed in the preparation of the bases.

Alternatively, the compound of formula (I) of the present invention may also be in the form of a pharmaceutically acceptable salt derived from an inorganic or organic base include salts obtained by adding an inorganic or organic base. For example, alkalis including sodium hydroxide or potassium hydroxide, or alkaline earth metal hydroxides including calcium hydroxide, magnesium hydroxide, aluminum hydroxide or ammonium hydroxide may be used for the preparation of inorganic salt of the compound. Further, organic bases including triethylamine or diisopropylethylamine may also be used for the preparation of organic salt of the compound.

The preferred inventive compound of formula (I-II) and (I-III) may be prepared as in Scheme (I).

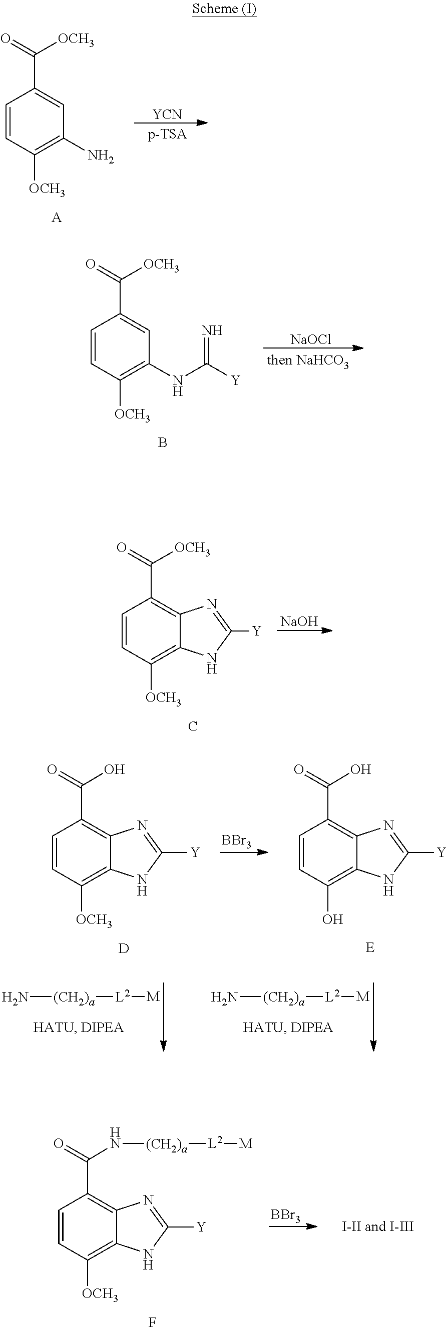

Wherein, p-TSA is p-toluenesulfonic acid, HATU is 2-(1H-7-Azabenzotriazol-1-yl)-1,1,3,3-tetramethyl uronium hexafluorophosphate Methanaminium, DIPEA is N,N-diisopropylethylamine and Y (except when Y is a hydrogen), a, L² and M have the same meaning as defined previously.

Aniline A is reacted with a nitrile in the presence of p-toluenesulfonic acid to afford amidine B. Amidine B is chlorinated with sodium hypochlorite and cyclized using sodium bicarbonate to form benzimidazole C. Intermediate C is saponified with sodium hydroxide to afford methoxy acid D. Compound D is treated with boron tribromide to afford hydroxy acid E. Hydroxy acid E is reacted with various amines using HATU to afford compounds of formula I-II. Compound D is also reacted with various amines in the presence of HATU to afford amides F. Amides F are treated with boron tribromide to afford compounds of formula (I-III).

The preferred inventive compound of formula (I-IV) can be prepared as shown in Scheme (II).

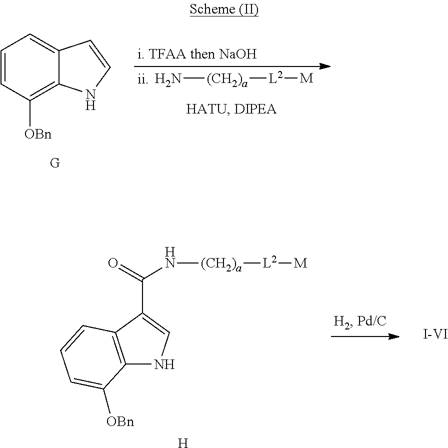

Compound G is reacted with TFAA (trifluoroacetic acid anhydride) followed by hydrolysis with base to afford the intermediate carboxylic acid, which is coupled using HATU to afford compound H. Compound H is hydrogenated to afford compounds of formula (I-VI).

The preferred inventive compound of formula (I-V) can be prepared as shown in Scheme (III).

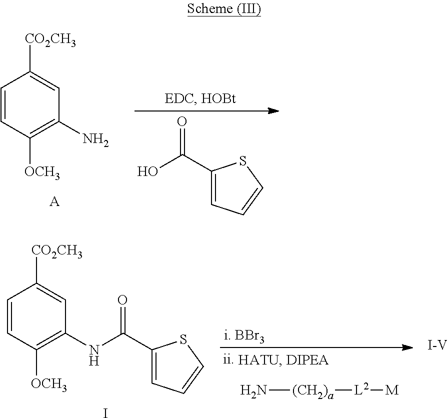

Aniline A is coupled with a carboxylic acid derivative to give the corresponding amide I. The ester and ether are cleaved with boron tribromide and the resulting acid is coupled with an amine derivative to give compounds of formula (I-V).

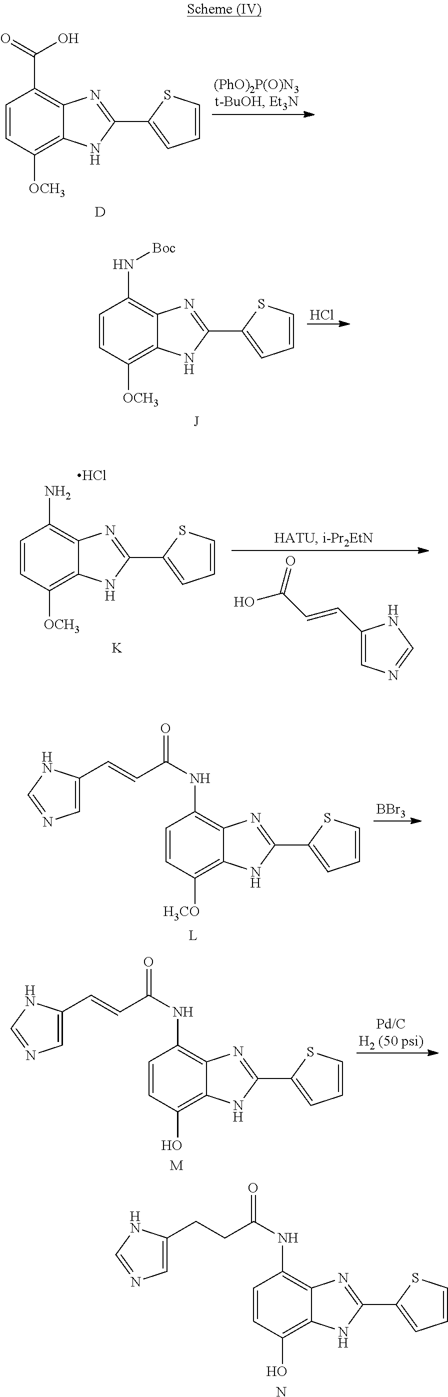

Acid D is treated with diphenylphosphoryl azide, triethyl amine and t-butanol to afford intermediate J. The boc-group is removed by treatment with hydrogen chloride to afford the amine K. Amine K is treated with the requisite acid in the presence of HATU to afford amide L. Compound L is reacted with boron tribromide to afford the phenol M. Compound M is treated with hydrogen in the presence of palladium to afford compound N (Scheme IV).

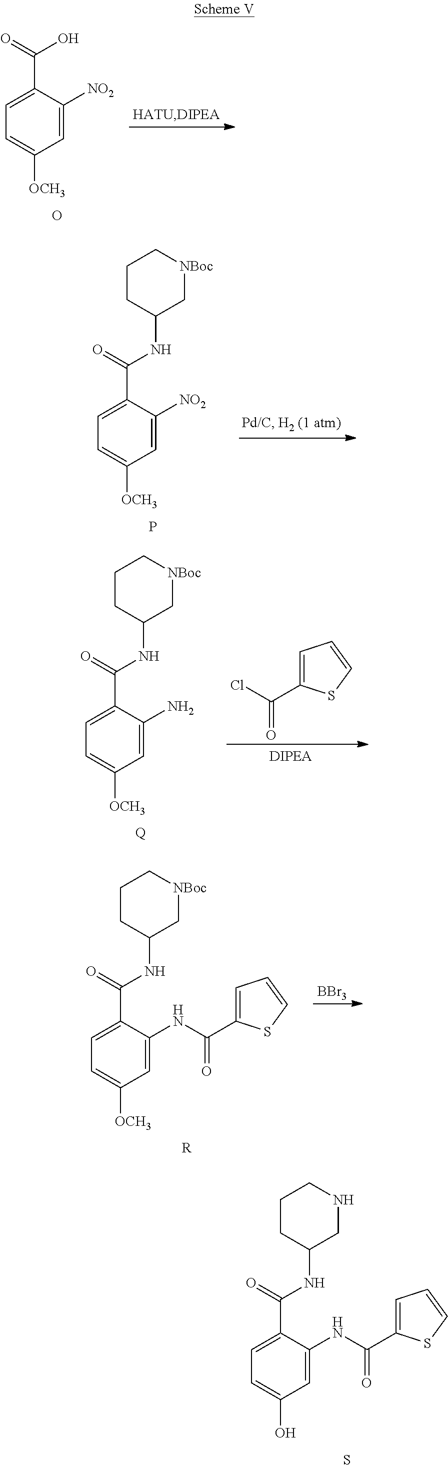

Acid O is coupled with the requisite amine to afford amide P. Compound P is reduced under standard hydrogenation conditions to afford aniline Q. The aniline is reacted with the requisite acid chloride to afford intermediate R. A final deprotection using boron tribromide affords compound S.

A salt, hydrate, solvate and isomer of the inventive compound of formula (I) may be prepared by employing any of the known methods. The inventive compound of formula (I), a salt, hydrate, solvate or isomer thereof may be used for the treatment of GSK3beta dependent diseases such as Alzheimer disease, mania, depression, migraine and type 2 diabetes, by way of inhibiting GSK3beta activity, the inventive compound having an IC₅₀ value (micro M), generally in the range of 0.0001 to 100, for example 0.001 to 50, preferably 0.001 to 10, more preferably 0.001 to 5.

Accordingly, the present invention includes a pharmaceutical composition which includes a therapeutically effective amount of the compound of formula (I), a salt, hydrate, solvate or isomer thereof as an active ingredient and a pharmaceutically acceptable carrier; therefore, the pharmaceutical composition of the present invention exerts superior preventive and treating effects on GSKbeta dependent diseases.

A pharmaceutical formulation may be prepared in accordance with any of the conventional procedures. In preparing the formulation, the active ingredient is preferably admixed or diluted with a carrier, or enclosed within a carrier, sachet or other container. When the carrier serves as a diluent, it may be a solid, semi-solid or liquid material acting as a vehicle, excipient or medium for the active ingredient. Thus, the formulations may be in the form of a tablet, pill, powder, sachet, elixir, suspension, emulsion, solution, syrup, aerosol, soft and hard gelatin capsule, sterile injectable solution, sterile packaged powder and the like.

Examples of suitable carriers, excipients, and diluents are lactose, dextrose, sucrose, sorbitol, mannitol, calcium silicate, cellulose, methyl cellulose, microcrystalline cellulose, polyvinylpyrrolidone, water, and mineral oil. The formulations may additionally include fillers, antiemulsifiers, preservatives and the like. The compositions of the invention may be formulated so as to provide quick, sustained or delayed release of the active ingredient after their administration to a mammal by employing any of the procedures well known in the art.

The pharmaceutical composition of the present invention can be administered via various routes including oral, transdermal, subcutaneous, intravenous and intramuscular introduction.

The dosage and method of administration vary according to the body-weight and age of a patient and the administration method; however, one skilled in the art can routinely select a suitable method of administration. If the compound is encodable by a DNA, the DNA can be inserted into a vector for gene therapy and the vector administered to a patient to perform the therapy. The dosage and method of administration vary according to the body-weight, age, and symptoms of the patient; however, one skilled in the art can suitably select them.

For example, although the dose of a compound of the present invention that regulates its activity depends on the symptoms, the dose is generally about 0.1 mg to about 100 mg per day, preferably about 1.0 mg to about 50 mg per day and more preferably about 1.0 mg to about 20 mg per day, when administered orally to a normal adult human (weight 60 kg).

When administering the compound parenterally, in the form of an injection to a normal adult human (weight 60 kg), although there are some differences according to the patient, target organ, symptoms and method of administration, it is convenient to intravenously inject a dose of about 0.01 mg to about 30 mg per day, preferably about 0.1 to about 20 mg per day, and more preferably about 0.1 to about 10 mg per day. In the case of other animals, the appropriate dosage amount may be routinely calculated by converting to 60 kg of body-weight.

### EXAMPLES

The following examples are intended to further illustrate the present invention without limiting its scope.

### Example 1

### Step 1: Synthesis of Methyl 4-Methoxy-3-(thiophene-2-carboximidamido)benzoate

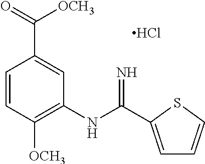

p-Toluenesulfonic acid monohydrate (42 g, 110 mmol) was heated at 120 degrees and once the solid completely melted, it was placed under high vacuum for 1 h to remove the water. The vacuum was released, aniline (20 g, 55 mmol) and 2-thiophenecarbonitrile (24 g, 110 mmol) were added, and the reaction mixture was heated at 160 degrees for 4 h. The reaction mixture was cooled to room temperature followed by addition of satd. aq NaHCO₃ (250 mL) and ethyl acetate (250 mL). The layers were separated, the aqueous layer was extracted with ethyl acetate (100 mL), and the combined organic layers were dried over Na₂SO₄, filtered, and concentrated. The crude residue was purified by column chromatography to obtain 16 g of the crude amidine intermediate. The crude intermediate was dissolved in ethyl acetate (350 mL) and HCl (2.0 M in diethyl ether, 55 mL, 110 mmol) was added. The resulting precipitate was filtered to obtain the desired product (16 g, 42% yield) as an off-white solid: ESI MS m/z 291 [C₁₄H₁₄N₃O₂S+H]⁺.

### Step 2: Synthesis of Methyl 7-Methoxy-2-(thiophen-2-yl)-1H-benzo[d]imidazole-4-carboxylate

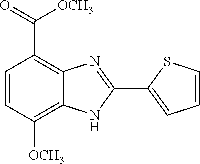

To a solution of the product from step 1 (16 g, 49 mmol) in methanol (100 mL) was added 5% aq NaOCl (75 mL, 55 mmol) and the reaction mixture was stirred at room temperature for 2 h. Next, satd. aq NaHCO₃ (150 mL) and methanol (150 mL) were added and the resulting reaction mixture was heated at 60 degrees for 2 d. The reaction mixture was cooled to room temperature and concentrated to remove methanol. The reaction mixture was acidified to pH 4 using 6 N HCl and the resulting precipitate was filtered and dried to obtain the desired product (8 g, 57% yield) as a brown solid: ¹H NMR (500 MHz, CDCl₃) delta 7.86 (d, J=8.5 Hz, 1H), 7.71-7.68 (m, 1H), 7.48-7.45 (m, 1H), 7.17-7.14 (m, 1H), 7.73 (d, J=8.5 Hz, 1H), 4.16 (m, 3H), 3.98 (m, 3H); ESI MS m/z 289 [C₁₄H₁₂N₂O₃S+H]⁺.

### Step 3: Synthesis of 7-Methoxy-2-(thiophen-2-yl)-1H-benzo[d]imidazole-4-carboxylic Acid

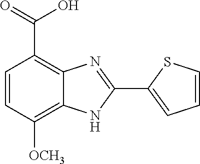

To a solution of the product from step 2 (4.2 g, 14 mmol) in ethanol (30 mL) and water (15 mL) was added 6 N NaOH (55 mL) and the reaction mixture was heated at 90 degrees for 2 h. The reaction mixture was cooled and concentrated to dryness. The crude residue was dissolved in water (30 ml) and acidified to pH 4 using 6 N HCl. The resulting precipitate was filtered and dried to obtain the desired product (2.2 g, 58% yield) as a brown solid: ¹H NMR (500 MHz, DMSO-d₆) delta 8.25 (d, J=3.0 Hz, 1H), 7.77 (d, J=8.0 Hz, 1H), 7.73-7.68 (m, 1H), 7.22-7.18 (m, 1H), 6.82 (d, J=8.5 Hz, 1H), 3.97 (m, 3H); ESI MS m/z 275 [C₁₃H₁₀N₂O₃S+H]⁺.

### Step 4: Synthesis of 7-Hydroxy-2-(thiophen-2-yl)-1H-benzo[d]imidazole-4-carboxylic Acid

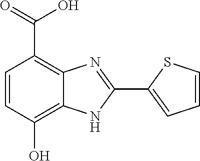

To a solution of the product from step 3 (2.5 g, 9.1 mmol) in dichloroethane (100 mL) was added BBr₃ (23 g, 91 mmol) and the reaction mixture was heated at 90 degrees for 2 d. The reaction mixture was cooled and poured onto ice. The resulting solids were filtered to obtain the desired product (0.45 g, 19% yield) as a brown solid. The filtrate was acidified to pH 4 using 6 N HCl and the resulting precipitate was filtered to obtain a second batch of the desired product (Example No. 1, 1.6 g, 88% yield) as a brown solid: ¹H NMR (300 MHz, CD₃OD) delta 7.93-7.90 (m, 1H), 7.75 (d, J=8.5 Hz, 1H), 7.62-7.58 (m, 1H), 7.19-7.14 (m, 1H), 6.65 (d, J=8.1 Hz, 1H); ESI MS m/z 261 [C₁₂H₈N₂O₃S+H]⁺.

### Example 2

### Step 1: Synthesis of Methyl 3-Methoxy-4-(thiophene-2-carboximidamido)benzoate Hydrochloride

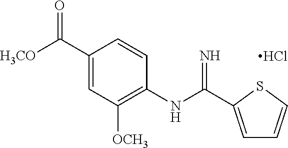

Following the procedure outlined for step 1 in Example 1, methyl 4-amino-3-methoxybenzoate (5.0 g, 27 mmol) was reacted with 2-thiophenecarbonitrile (4.4 g, 41 mmol) to afford the desired product (4.5 g, 50% yield) as a brown solid: ESI MS m/z 291 [C₁₄H₁₄N₂O₃S+H]⁺.

### Step 2: Synthesis of Methyl 7-methoxy-2-(thiophen-2-yl)-1H-benzo[d]imidazole-5-carboxylate

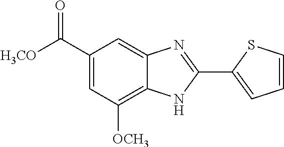

Following the procedure outlined for step 2 in Example 1, methyl 3-methoxy-4-(thiophene-2-carboximidamido)benzoate hydrochloride (4.5 g, 13 mmol) was reacted with NaOCl followed by satd. aq NaHCO₃ to afford the desired product (3.1 g, 78% yield) as a brown solid: ¹H NMR (300 MHz, DMSO-d₆) delta 13.50 (s, 1H), 13.27 (s, tautomer), 8.05-7.72 (m, 3H), 7.36-7.22 (m, 2H), 4.02 (s, 3H), 3.94 (s, 3H); ESI MS m/z 289 [C₁₄H₁₂N₂O₃S+H]⁺.

### Step 3: Synthesis of 7-Methoxy-2-(thiophen-2-yl)-1H-benzo[d]imidazole-5-carboxylic Acid

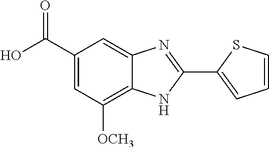

Following the procedure outlined for step 3 in Example 1, methyl 7-methoxy-2-(thiophen-2-yl)-1H-benzo[d]imidazole-5-carboxylate (1.5 g, 5.4 mmol) was reacted with sodium hydroxide to afford the desired product (quant.) as a brown solid: ¹H NMR (300 MHz, DMSO-d₆) delta 8.05 (s, J=3.0 Hz, 1H), 7.83 (d, J=4.8 Hz, 1H), 7.80 (s, 1H), 7.35 (s, 1H), 7.29-7.26 (m, 1H), 4.01 (s, 3H); ESI MS m/z 275 [C₁₃H₁₀N₂O₃S+H]⁺.

### Example 3

### Step 1: Synthesis of Methyl 3-(Furan-2-carboximidamido)-4-methoxybenzoate Hydrochloride

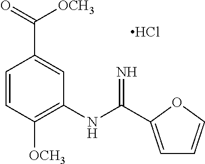

Following the procedure outlined for step 1 in Example 1, methyl 3-amino-4-methoxybenzoate (10 g, 55.2 mmol) was reacted with 2-furylcarbonitrile (8.0 g, 86 mmol) to afford the desired product (8.5 g, 49% yield) as an off-white solid: ESI MS m/z 275 [C₁₄H₁₄N₃O₄+H]⁺.

### Step 2: Synthesis of 2-(Furan-2-yl)-7-methoxy-1H-benzo[d]imidazole-4-carboxylic Acid

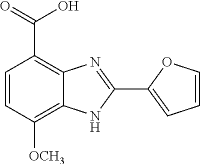

To a solution of methyl 3-(furan-2-carboximidamido)-4-methoxybenzoate Hydrochloride (8.5 g, 27 mmol) in methanol (60 mL) was added 5% aq NaOCl (60 mL, 41 mmol) and the reaction mixture was stirred at room temperature for 2 h. Next, satd. aq NaHCO₃ (70 mL) and methanol (60 mL) were added and the resulting reaction mixture was heated at 90 degrees for 16 h. Then, 6 N NaOH (50 mL, 300 mmol) was added and the reaction mixture was heated at 90 degrees for an additional 3 h. The reaction mixture was cooled to room temperature and concentrated to remove methanol. The reaction mixture was acidified to pH 5 using 6 N HCl and the resulting precipitate was filtered and dried to afford desired product (4.0 g, 57% yield) as a brown solid: ESI MS m/z 261 [C₁₃H₁₀N₂O₄+H]⁺.

### Step 3: Synthesis 2-(Furan-2-yl)-7-hydroxy-1H-benzo[d]imidazole-4-carboxylic Acid

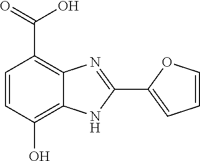

Following the procedure outlined for step 4 in Example 1, 2-(Furan-2-yl)-7-methoxy-1H-benzo[d]imidazole-4-carboxylic acid (2.0 g, 7.7 mmol) was reacted with boron tribromide (15 g, 60 mmol) to afford the desired product (1.2 g, 63% yield) as a brown solid: ESI MS m/z 245 [C₁₂H₈N₂O₄+H]⁺.

### Example 4

### Step 1: Synthesis of Methyl 4-fluoro-3-(thiophene-2-carboximidamido)benzoate Hydrochloride

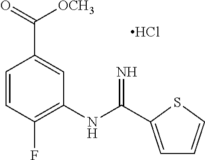

Following the procedure outlined for step 1 in Example, methyl 3-amino-4-fluorobenzoate (5 g, 29.6 mmol) was reacted with 2-thiophenecarbonitrile (6.5 g, 59.2 mmol) to afford the desired product (1.8 g) as a light brown solid: ESI MS m/z 279 [C₁₃H₁₁FN₂O₂S+H]⁺.

### Step 2: Synthesis of Methyl 7-fluoro-2-(thiophen-2-yl)-1H-benzo[d]imidazole-4-carboxylate

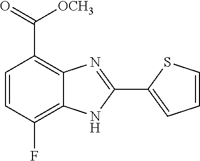

Following the procedure outlined for step 2 in Example 1, methyl 4-fluoro-3-(thiophene-2-carboximidamido)benzoate hydrochloride (1.7 g, 6.0 mmol) was reacted with 5% aq NaOCl and satd. aq NaHCO₃ to afford the desired product (0.21 g, 3% yield) as a yellow solid: ESI MS m/z 277 [C₁₃H₉FN₂O₂S+H]⁺.

### Step 3: Synthesis of 7-Fluoro-2-(thiophen-2-yl)-1H-benzo[d]imidazole-4-carboxylic Acid

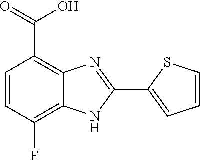

Following the procedure outlined for step 4 in Example 1, methyl 7-fluoro-2-(thiophen-2-yl)-1H-benzo[d]imidazole-4-carboxylate (0.2 g, 0.7 mmol) was reacted with 3 N NaOH (10 mL) to afford the desired product (0.1 g crude) as an off-white solid: ESI MS m/z 263 [C₁₂H₇FN₂O₂S+H]⁺.

### Example 5

### Step 1: Synthesis of Methyl 3-(Cyclopropanecarboximidamido)-4-methoxybenzoate Hydrochloride

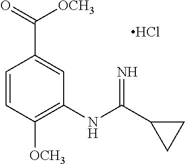

Following the procedure outlined for step 1 in Example 1, methyl 3-amino-4-methoxybenzoate (10 g, 55 mmol) was reacted with cyclopropanecarbonitrile (7.4 g, 110 mmol) to afford the desired product (16 g crude) as a black solid: ESI MS m/z 249 [C₁₃H₁₆N₂O₃+H]⁺.

### Step 2: Synthesis of Methyl 2-Cyclopropyl-7-methoxy-1H-benzo[d]imidazole-4-carboxylate

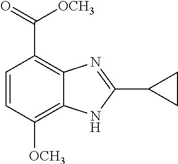

Following the procedure outlined for step 2 in Example 1, methyl 3-(cyclopropanecarboximidamido)-4-methoxybenzoate hydrochloride (15 g, 50 mmol) was reacted with aq NaOCl followed by satd. aq NaHCO₃ to afford the desired product (12 g crude) as a brown solid: ESI MS m/z 247 [C₁₃H₁₄N₂O₃+H]⁺.

### Step 3: Synthesis of 2-Cyclopropyl-7-methoxy-1H-benzo[d]imidazole-4-carboxylic Acid

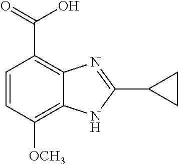

Following the procedure outlined for step 3 in Example 1, methyl 2-cyclopropyl-7-methoxy-1H-benzo[d]imidazole-4-carboxylate (2.0 g, 8.0 mmol) was reacted with sodium hydroxide to afford the desired product (1.7 g crude) as a black solid: ESI MS m/z 233 [C₁₂H₁₂N₂O₃+H]⁺.

### Step 4: Synthesis of 2-Cyclopropyl-7-hydroxy-1H-benzo[d]imidazole-4-carboxylic Acid

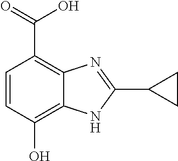

Following the procedure outlined for step 4 in Example 1, 2-cyclopropyl-7-methoxy-1H-benzo[d]imidazole-4-carboxylic acid (1.5 g, 6.1 mmol) was reacted with boron tribromide to afford the desired product (1.2 g crude) as a black solid: ESI MS m/z 219 [C₁₁H₁₀N₂O₃+H]⁺.

### Example 6

### Step 1: Synthesis of Methyl 3-(Cyclopentanecarboximidamido)-4-methoxybenzoate Hydrochloride

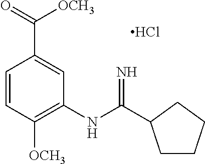

Following the procedure outlined for step 1 in Example 1, methyl 3-amino-4-methoxybenzoate (5.0 g, 27 mmol) was reacted with cyclopentanecarbonitrile (5.2 g, 55 mmol) to afford the desired product (7.7 g crude) as a brown solid: ESI MS m/z 277 [C₁₅H₂₀N₂O₃+H]⁺.

### Step 2: Synthesis of Methyl 2-Cyclopentyl-7-methoxy-1H-benzo[d]imidazole-4-carboxylate

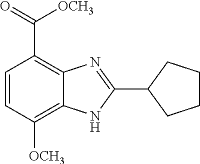

Following the procedure outlined for step 2 in Example 1, methyl 3-(cyclopentanecarboximidamido)-4-methoxybenzoate hydrochloride (5.6 g, 18 mmol) was reacted with aq NaOCl followed by satd. aq NaHCO₃ to afford the desired product (4.9 g crude) as a black solid: ESI MS m/z 275 [C₁₅H₁₈N₂O₃+H]⁺.

### Step 3: Synthesis of 2-Cyclopentyl-7-hydroxy-1H-benzo[d]imidazole-4-carboxylic Acid

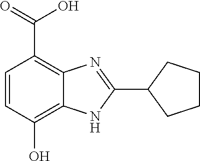

Following the procedure outlined for step 4 in Example 1, methyl 2-cyclopentyl-7-methoxy-1H-benzo[d]imidazole-4-carboxylate (1.1 g, 4.0 mmol) was reacted with boron tribromide to afford the desired product (0.92 g crude) as a black solid: ESI MS m/z 247 [C₁₃H₁₄N₂O₃+H]⁺.

### Example 7

### Step 1: Synthesis of Methyl 3-Benzimidamido-4-methoxybenzoate Hydrochloride

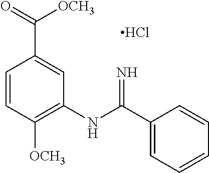

Following the procedure outlined for step 1 in Example 1, methyl 3-amino-4-methoxybenzoate (5.0 g, 27 mmol) was reacted with benzonitrile (5.7 g, 55 mmol) to afford the desired product (7.8 g crude) as a black solid: ESI MS m/z 285 [C₁₆H₁₆N₂O₃+H]⁺.

### Step 2: Synthesis of Methyl 7-Methoxy-2-phenyl-1H-benzo[d]imidazole-4-carboxylate

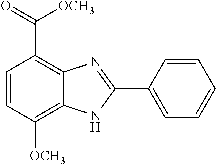

Following the procedure outlined for step 2 in Example 1, methyl 3-benzimidamido-4-methoxybenzoate hydrochloride (2.0 g, 8.0 mmol) was reacted with aq NaOCl followed by satd. aq NaHCO₃ to afford the desired product (1.7 g crude) as an off-white solid: ESI MS m/z 283 [C₁₆H₁₄N₂O₃+H]⁺.

### Step 3: Synthesis of 7-Hydroxy-2-phenyl-1H-benzo[d]imidazole-4-carboxylic Acid

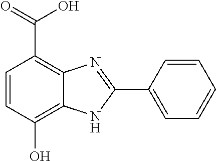

Following the procedure outlined for step 4 in Example 1, methyl 7-methoxy-2-phenyl-1H-benzo[d]imidazole-4-carboxylate (4.0 g, 12 mmol) was reacted with boron tribromide to afford the desired product (2.1 g, crude) as a black solid: ESI MS m/z 255 [C₁₄H₁₀N₂O₃+H]⁺.

### General Procedure A—Synthesis of Compounds of Formula I-II as Described in Scheme (1):

To a solution of acid (1.0 equiv) in DMF (5-10 mL) was added HATU (1.2-1.5 equiv), DIPEA (3.0-5.0 equiv), and the amine (1.5-2.0 equiv) and the reaction mixture was either stirred at room temperature for 16 h or heated at 50-70 degrees for 16 h. The reaction mixture was diluted with satd. aq NaHCO₃ (20 mL) and extracted with ethyl acetate (3×20 mL). The combined organic layer was dried over Na₂SO₄, concentrated, and purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products. In some instances the desired product was treated with TFA (1-2 mL) for 1 h, concentrated and purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products

### Example 8

### 7-Hydroxy-N-(4-sulfamoylbenzyl)-2-(thiophen-2-yl)-1H-benzo[d]imidazole-4-carboxamide

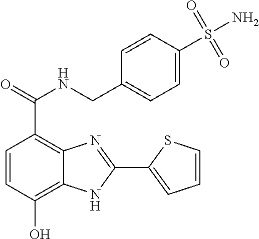

Following General Procedure A,

7-methoxy-2-(thiophen-2-yl)-1H-benzo[d]imidazole-4-carboxylic acid (125 mg, 0.36 mmol) was reacted with 4-(aminomethyl)benzenesulfonamide (0.13 g, 0.72 mmol) to afford the desired product (30 mg, 19% yield) as a light yellow solid: ¹H NMR (300 MHz, CD₃OD) delta 7.94-7.79 (m, 4H), 7.67-7.59 (m, 3H), 7.20-7.16 (m, 1H), 6.71 (d, J=8.1 Hz, 1H), 4.82 (s, 2H); ESI MS m/z 429 [C₁₉H₁₆N₄O₄S₂+H]⁺; HPLC 98.4% (AUC), t_{R}=11.94 min.

### Example 9

### N-(2,4-Difluorobenzyl)-7-hydroxy-2-(thiophen-2-yl)-1H-benzo[d]imidazole-4-carboxamide

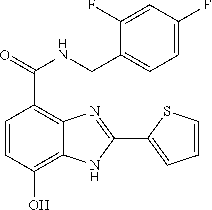

Following General Procedure A,

7-methoxy-2-(thiophen-2-yl)-1H-benzo[d]imidazole-4-carboxylic acid (125 mg, 0.36 mmol) was reacted with (2,4-difluorophenyl)methanamine (0.10 g, 0.72 mmol) to afford the desired product (33 mg, 24% yield) as an off-white solid: ¹H NMR (300 MHz, CD₃OD) delta 7.85-7.78 (m, 2H), 7.62-7.57 (m, 2H), 7.20-7.17 (m, 1H), 7.01-6.95 (m, 2H), 6.71 (d, J=8.4 Hz, 1H), 4.75 (s, 2H); ESI MS m/z 368 [C₁₉H₁₃F₂N₃O₂S+H]⁺; HPLC 96.2% (AUC), t_{R}=14.47 min.

### Example 10

### 7-Hydroxy-N-(thiazol-2-yl)-2-(thiophen-2-yl)-1H-benzo[d]imidazole-4-carboxamide

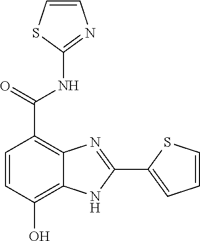

Following General Procedure A,

7-methoxy-2-(thiophen-2-yl)-1H-benzo[d]imidazole-4-carboxylic acid (146 mg, 0.43 mmol) was reacted with thiazol-2-amine (0.072 g, 0.72 mmol) to afford the desired product (15 mg, 10% yield) as a light brown solid: ¹H NMR (300 MHz, CD₃OD) delta 8.02-8.00 (m, 1H), 7.93 (d, J=8.4 Hz, 1H), 7.69 (d, J=5.1 Hz, 1H), 7.54-7.53 (m, 1H), 7.25-7.17 (m, 2H), 6.79 (d, J=8.4 Hz, 1H); ESI MS m/z 343 [C₁₅H₁₀N₄O₂S₂+H]⁺; HPLC>99% (AUC), t_{R}=14.10 min.

### Example 11

### 7-Hydroxy-N-[2-(pyridin-2-ylamino)ethyl]-2-(thiophen-2-yl)-1H-benzo[d]imidazole-4-carboxamide

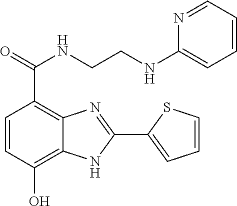

Following General Procedure A,

7-methoxy-2-(thiophen-2-yl)-1H-benzo[d]imidazole-4-carboxylic acid (0.24 g, 0.91 mmol) was reacted with N¹-(pyridin-2-yl)ethane-1,2-diamine (0.098 g, 0.72 mmol) to afford the desired product (114 mg, 33% yield) as a white solid: ¹H NMR (500 MHz, DMSO-d₆) 8.00-7.96 (m, 2H), 7.72-7.70 (m, 2H), 7.36 (dd, J=3.0, 1.5 Hz, 1H), 7.21 (t, J=4.0 Hz, 1H), 6.73 (d, J=8.5 Hz, 1H), 6.53 (d, J=8.5 Hz, 1H), 6.48 (t, J=1.0 Hz, 1H), 3.62 (t, J=6.5 Hz, 2H), 3.48 (t, J=6.5 Hz, 2H); ESI MS m/z 380 [C₁₉H₁₇N₅O₂S+H]⁺; HPLC>99% (AUC), t_{R}=11.12 min.

### Example 12

### 7-Hydroxy-N-[3-(2-hydroxyethylamino)propyl]-2-(thiophen-2-yl)-1H-benzo[d]imidazole-4-carboxamide

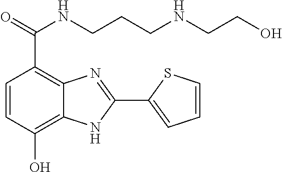

Following General Procedure A,

7-methoxy-2-(thiophen-2-yl)-1H-benzo[d]imidazole-4-carboxylic acid (0.15 g, 0.58 mmol) was reacted with 2-(3-aminopropylamino)ethanol (0.084 g, 0.72 mmol) to afford the desired product (31 mg, 15% yield) as a yellow-brown solid: ¹H NMR (500 MHz, CD₃OD) 7.86 (dd, J=3.5, 1.0 Hz, 1H), 7.76 (d, J=8.5 Hz, 1H), 7.62 (dd, J=5.0, 1.0 Hz, 1H), 6.66 (d, J=8.5 Hz, 1H), 3.73 (t, J=5.0 Hz, 2H), 3.64 (t, J=6.5 Hz, 2H), 2.99 (t, J=7.0 Hz, 2H), 2.94 (t, J=5.5 Hz, 2H), 2.02-1.99 (m, 2H); ESI MS m/z 361 [C₁₇H₂₀N₄O₃S+H]⁺.

### Example 13

### (S)-Methyl 2-[7-Hydroxy-2-(thiophen-2-yl)-1H-benzo[d]imidazole-4-carboxamido]-3-(1H-imidazol-5-yl)propanoate

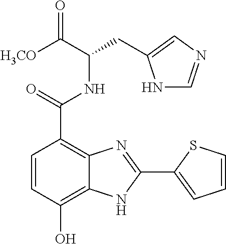

Following General Procedure A,

7-methoxy-2-(thiophen-2-yl)-1H-benzo[d]imidazole-4-carboxylic acid (150 mg, 0.57 mmol) was reacted with (S)-methyl 2-amino-3-(1H-imidazol-5-yl)propanoate (0.12 g, 0.72 mmol) to afford the desired product (66 mg, 23% yield) as a light yellow solid: ¹H NMR (500 MHz, CD₃OD) delta 7.85 (d, J=3.6 Hz, 1H), 7.74 (d, J=8.3 Hz, 1H), 7.62 (d, J=4.2 Hz, 1H) 7.58 (s, 1H) 7.19 (t, J=4.9 Hz, 1H), 7.03 (s, 1H), 6.69 (d, J=8.3 Hz, 1H) 5.01-4.99 (m, 1H), 3.77 (s, 3H); ESI MS m/z 412 [C₁₉H₁₇N₅O₄S+H]⁺; HPLC 96.3% (AUC), t_{R}=7.94 min.

### Example 14

### (S)-Methyl 2-[7-Hydroxy-2-(thiophen-2-yl)-1H-benzo[d]imidazole-4-carboxamido]-3-(1H-indol-3-yl)propanoate

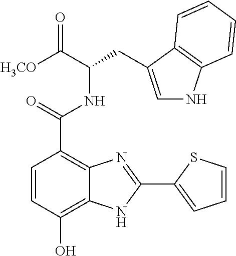

Following General Procedure A,

7-methoxy-2-(thiophen-2-yl)-1H-benzo[d]imidazole-4-carboxylic acid (150 mg, 0.57 mmol) was reacted with (S)-methyl 2-amino-3-(1H-indol-3-yl)propanoate (0.16 g, 0.72 mmol) to afford the desired product (65 mg, 13% yield) as a light yellow solid: ¹H NMR (500 MHz, CD₃OD) delta 7.77-7.58 (m, 2H), 7.58-7.56 (m, 2H), 7.28 (d, J=6.0 Hz, 2H), 7.16 (t, J=4.9 Hz, 1H), 7.04 (t, J=7.5 Hz, 1H), 6.94 (t, J=15.1 Hz, 1H), 6.68 (d, J=8.3 Hz, 1H), 5.04 (t, J=6.3 Hz, 1H), 3.69 (s, 3H) 3.45 (d, J=6.25 Hz, 2H); ESI MS m/z 461 [C₂₄H₂₀N₄O₄S+H]⁺; HPLC 98.7% (AUC), t_{R}=13.03 min.

### Example 15

### Methyl 3-Hydroxy-2-[7-hydroxy-2-(thiophen-2-yl)-1H-benzo[d]imidazole-4-carboxamido]propanoate

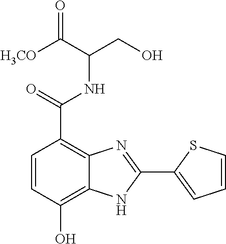

Following General Procedure A,

7-methoxy-2-(thiophen-2-yl)-1H-benzo[d]imidazole-4-carboxylic acid (150 mg, 0.57 mmol) was reacted with methyl 2-amino-3-hydroxypropanoate (0.084 g, 0.72 mmol) to afford the desired product (20 mg, 10% yield) as a light yellow solid: ¹H NMR (500 MHz, CD₃OD) delta 7.94 (d, J=3.2 Hz, 1H), 7.80 (d, J=8.3 Hz, 1H), 7.67 (d, J=4.8 Hz, 1H), 7.21 (d, J=4.8 Hz, 1H), 6.73 (d, J=8.3 Hz, 1H), 4.11-4.05 (m, 1H), 4.01-3.98 (m, 1H), 3.82 (s, 3H); ESI MS m/z 362 [C₁₆H₁₅N₃O₅S+H]⁺; HPLC 95.0% (AUC), t_{R}=11.36 min.

### Example 16

### Methyl 2-[7-Hydroxy-2-(thiophen-2-yl)-1H-benzo[d]imidazole-4-carboxamido]-3-(4-hydroxyphenyl)propanoate

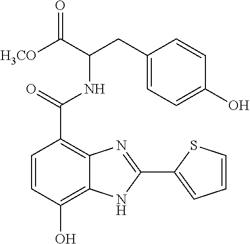

Following General Procedure A,

7-methoxy-2-(thiophen-2-yl)-1H-benzo[d]imidazole-4-carboxylic acid (150 mg, 0.57 mmol) was reacted with methyl 2-amino-3-(4-hydroxyphenyl)propanoate (0.14 g, 0.72 mmol) to afford the desired product (15 mg, 6% yield) as a light yellow solid: ¹H NMR (500 MHz, DMSO-d₆) delta 13.45 (s, 1H), 10.86 (s, 1H), 9.85 (d, J=6.8 Hz, 1H), 9.18 (s, 1H), 8.07 (d, J=3.6 Hz, 1H), 7.81 (d, J=5.0 Hz, 1H), 7.65 (d, J=8.3 Hz, 1H), 7.27 (t, J=4.9 Hz, 1H), 7.16 (d, J=8.4 Hz, 2H), 6.72 (d, J=8.2 Hz, 1H), 6.67 (d, J=8.4 Hz, 2H), 4.71-4.68 (m, 1H), 3.64 (s, 3H), 3.16-3.10 (m, 1H), 3.01-2.96 (m, 1H); ESI MS m/z 362 [C₁₆H₁₅N₃O₅S+H]⁺; HPLC 95.0% (AUC), t_{R}=11.36 min.

### Example 17

### (R)-7-Hydroxy-N-[1-hydroxy-3-(1H-imidazol-4-yl)propan-2-yl]-2-(thiophen-2-yl)-1H-benzo[d]imidazole-4-carboxamide

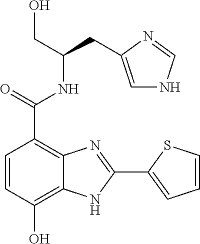

Following General Procedure A,

7-methoxy-2-(thiophen-2-yl)-1H-benzo[d]imidazole-4-carboxylic acid (150 mg, 0.57 mmol) was reacted with (R)-2-amino-3-(1H-imidazol-4-yl)propan-1-ol (0.10 g, 0.72 mmol) to afford the desired product (41 mg, 18% yield) as a light yellow solid: ¹H NMR (500 MHz, CD₃OD) delta 7.86 (d, J=3.6 Hz, 1H), 7.73 (d, J=8.3 Hz, 1H), 7.62 (d, J=5.0 Hz, 1H), 7.59 (s, 1H), 7.20-7.18 (m, 1H), 6.96 (s, 1H), 6.68 (d, 1H), 4.44-4.41 (m, 1H), 3.75 (d, J=4.8 Hz, 2H), 3.16-3.09 (m, 1H), 3.04-3.00 (m, 1H); ESI MS m/z 383 [C₁₈H₁₇N₅O₃S+H]⁺.

### Example 18

### (S)-7-Hydroxy-N-(1-hydroxy-3,3-dimethylbutan-2-yl)-2-(thiophen-2-yl)-1H-benzo[d]imidazole-4-carboxamide

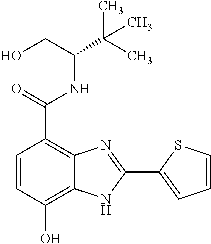

Following General Procedure A,

7-methoxy-2-(thiophen-2-yl)-1H-benzo[d]imidazole-4-carboxylic acid (150 mg, 0.57 mmol) was reacted with (S)-2-amino-3,3-dimethylbutan-1-ol (0.084 g, 0.72 mmol) to afford the desired product (24 mg, 12% yield) as a light yellow solid: ¹H NMR (500 MHz, CD₃OD) delta 7.86 (d, J=3.6 Hz, 1H), 7.80 (d, J=8.3 Hz, 1H), 7.60 (d, J=5.0 Hz, 1H), 7.19 (t, J=5.0 Hz, 1H), 6.71 (d, J=8.3 Hz, 1H), 4.08-4.06 (m, 1H), 3.96-3.93 (m, 1H), 3.73-3.69 (m, 1H), 1.14 (s, 9H); ESI MS m/z 362 [C₁₈H₂₁N₃O₃S+H]⁺; HPLC>99% (AUC), t_{R}=13.85 min.

### Example 19

### (S)-7-Hydroxy-N-(1-hydroxy-3-phenylpropan-2-yl)-2-(thiophen-2-yl)-1H-benzo[d]imidazole-4-carboxamide

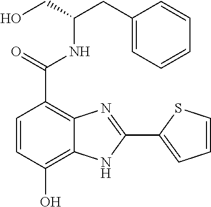

Following General Procedure A,

7-methoxy-2-(thiophen-2-yl)-1H-benzo[d]imidazole-4-carboxylic acid (200 mg, 0.73 mmol) was reacted with (S)-2-amino-3-phenylpropan-1-ol (0.11 g, 0.72 mmol) to afford the desired product (55 mg, 19% yield) as a light yellow solid: ¹H NMR (500 MHz, CD₃OD) delta 7.87 (d, J=3.7 Hz, 1H), 7.73 (d, J=8.3 Hz, 1H), 7.63 (d, J=4.0 Hz, 1H), 7.38 (d, J=7.1 Hz, 2H), 7.21-7.19 (m, 3H), 7.11 (t, J=13.6 Hz, 1H), 6.67 (d, J=8.3 Hz, 1H), 4.40-4.37 (m, 1H), 3.75-3.68 (m, 2H), 3.16-3.12 (m, 1H) 3.01-2.97 (m, 1H); ESI MS m/z 394 [C₂₁H₁₉N₃O₃S+H]⁺; HPLC>99% (AUC), t_{R}=13.67 min.

### Example 20

### 7-Hydroxy-2-(thiophen-2-yl)-N-[2-(thiophen-2-yl)ethyl]-1H-benzo[d]imidazole-4-carboxamide

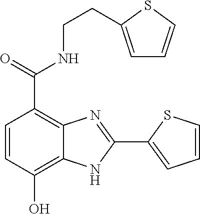

Following General Procedure A,

7-methoxy-2-(thiophen-2-yl)-1H-benzo[d]imidazole-4-carboxylic acid (150 mg, 0.577 mmol) was reacted with 2-(thiophen-2-yl)ethanamine (0.087 g, 0.72 mmol) to afford the desired product (42 mg, 20% yield) as a light yellow solid: ¹H NMR (500 MHz, CD₃OD) delta 7.82 (d, J=3.3 Hz, 1H) 7.79 (d, J=8.3 Hz, 1H), 7.62-7.61 (m, 1H), 7.18 (t, J=9.9 Hz, 2H), 6.99 (s, 1H), 6.91 (t, J=8.6 Hz, 1H), 6.69 (d, J=8.3 Hz, 1H), 3.81 (t, J=6.7 Hz, 2H), 3.23 (t, J=6.7 Hz, 2H); ESI MS m/z 370 [C₁₈H₁₅N₃O₂S₂+H]⁺; HPLC>99% (AUC), t_{R}=12.80 min.

### Example 21

### 7-Hydroxy-N-(4-sulfamoylphenethyl)-2-(thiophen-2-yl)-1H-benzo[d]imidazole-4-carboxamide

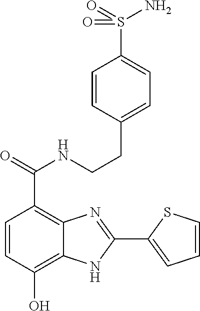

Following General Procedure A,

7-methoxy-2-(thiophen-2-yl)-1H-benzo[d]imidazole-4-carboxylic acid (150 mg, 0.577 mmol) was reacted with 4-(2-aminoethyl)benzenesulfonamide (0.14 g, 0.72 mmol) to obtain the desired product (18.3 mg, 7%) as an off-white solid: ¹H NMR (500 MHz, CD₃OD) delta 7.83-7.81 (m, 3H), 7.76 (d, J=8.1 Hz, 1H) 7.62 (d, J=4.8 Hz, 1H), 7.54 (d, J=7.6 Hz, 2H), 7.18 (t, J=8.8 Hz, 1H), 6.68 (d, J=8.2 Hz, 1H), 3.85 (t, J=11.5 Hz, 2H), 3.11 (t, J=6.1 Hz, 2H); ESI MS m/z 443 [C₂₀H₁₈N₄O₄S₂+H]⁺; HPLC>99% (AUC), t_{R}=12.14 min.

### Example 22

### 7-Hydroxy-N-(3-methoxyphenethyl)-2-(thiophen-2-yl)-1H-benzo[d]imidazole-4-carboxamide

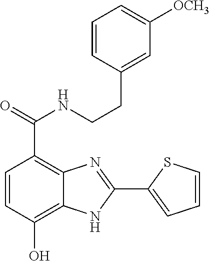

Following General Procedure A,

7-methoxy-2-(thiophen-2-yl)-1H-benzo[d]imidazole-4-carboxylic acid (150 mg, 0.58 mmol) was reacted with 2-(3-methoxyphenyl)ethanamine (0.11 g, 0.72 mmol) to obtain the desired product (24 mg, 11% yield) as a light yellow solid: ¹H NMR (500 MHz, DMSO-d₆) delta 8.14 (d, J=0.5 Hz, 1H), 8.00 (d, J=1.0 Hz, 1H), 7.78 (d, J=4.0 Hz, 1H), 7.77-7.17 (m, 2H), 6.93-6.82 (m, 2H), 6.75-6.70 (m, 1H). 3.73-3.65 (m, 5H), 2.92-2.82 (m, 2H); ESI MS m/z 394 [C₂₁H₁₉N₃O₃S+H]⁺; HPLC 95.7% (AUC), t_{R}=14.24 min.

### Example 23

### 7-hydroxy-N-(3-methoxyphenethyl)-2-(thiophen-2-yl)-1H-benzo[d]imidazole-4-carboxamide

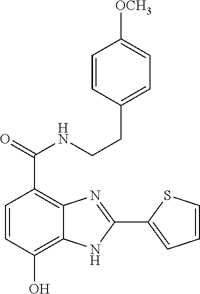

Following General Procedure A,

7-methoxy-2-(thiophen-2-yl)-1H-benzo[d]imidazole-4-carboxylic acid (150 mg, 0.58 mmol) was reacted with 2-(4-methoxyphenyl)ethanamine (0.11 g, 0.72 mmol) to obtain the desired product (30 mg, 9% yield) as a light yellow solid: ¹H NMR (500 MHz, DMSO-d₆) 8.01 (d, J=3.5 Hz, 1H), 7.81 (s, 1H), 7.77 (d, J=5.0, 1H), 7.26-7.23 (m, 3H), 6.86-6.82 (m, 2H), 6.73-6.70 (m, 2H), 3.71-3.63 (m, 5H), 2.87-2.63 (m, 2H); ESI MS m/z 394 [C₂₁H₁₉N₃O₃S+H]⁺; HPLC 94.5% (AUC), t_{R}=14.29 min.

### General Procedure B—Synthesis of Amides F as Described in Scheme (1):

To a suspension of 7-methoxy-2-(thiophen-2-yl)-1H-benzo[d]imidazole-4-carboxylic acid (1.0 equiv) in toluene (5-15 mL) was added thionyl chloride (4.0 equiv). After stirring at room temperature for 16 h, the reaction mixture was heated at 70 degrees for 2 h. The reaction mixture was cooled, and concentrated, and the residue was suspended in THF (10-20 mL) followed by the addition of pyridine (2.0 equiv) and the corresponding amine (2.0 equiv) and the reaction mixture was heated at 70 degrees for 16 h. The reaction mixture was concentrated and the residue was diluted with water (20 mL) and extracted with ethyl acetate (3×20 mL). The combined organic layers were washed with satd. aq NaHCO₃ (20 mL), concentrated, and purified by flash chromatography (silica, 0-15% methanol/dichloromethane) to afford amides F. In most cases these intermediates were isolated as crude products and were carried forward without extensive characterization or further purification.

### Example 24

### 7-Methoxy-N-[2-(1-methyl-1H-imidazol-5-yl)ethyl]-2-(thiophen-2-yl)-1H-benzo[d]imidazole-4-carboxamide

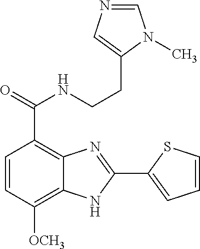

Following General Procedure B,

7-methoxy-2-(thiophen-2-yl)-1H-benzo[d]imidazole-4-carboxylic acid (170 mg, 0.62 mmol) was reacted with 2-(1-methyl-1H-imidazol-5-yl)ethanamine (0.15 g, 1.2 mmol) to afford the desired product (170 mg) as a yellow solid: ESI MS m/z 382 [C₁₉H₁₉N₅O₂S+H]⁺.

### Example 25

### N-[2-(Dimethylamino)ethyl]-7-methoxy-2-(thiophen-2-yl)-1H-benzo[d]imidazole-4-carboxamide

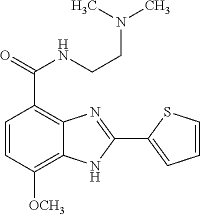

Following General Procedure B,

7-methoxy-2-(thiophen-2-yl)-1H-benzo[d]imidazole-4-carboxylic acid (160 mg, 0.58 mmol) was reacted with N¹,N¹-dimethylethane-1,2-diamine (0.10 g, 1.2 mmol) to afford the desired product (136 mg) as a brown glass: ESI MS m/z 345 [C₁₇H₂₀N₄O₂S+H]⁺.

### Example 26

### N-(3,4-Dimethoxybenzyl)-7-methoxy-2-(thiophen-2-yl)-1H-benzo[d]imidazole-4-carboxamide

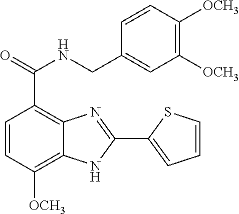

Following General Procedure B,

7-methoxy-2-(thiophen-2-yl)-1H-benzo[d]imidazole-4-carboxylic acid (158 mg, 0.58 mmol) was reacted with (3,4-dimethoxyphenyl)methanamine (0.20 g, 1.2 mmol) to afford the desired product (248 mg) as a brown solid: ESI MS m/z 424 [C₂₂H₂₁N₃O₄S+H]⁺.

### Example 27

### 7-Methoxy-N-[2-(phenylsulfonamido)ethyl]-2-(thiophen-2-yl)-1H-benzo[d]imidazole-4-carboxamide

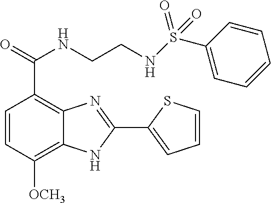

Following General Procedure B,

7-methoxy-2-(thiophen-2-yl)-1H-benzo[d]imidazole-4-carboxylic acid (0.18 g, 0.65 mmol) was reacted with N-(2-aminoethyl)benzenesulfonamide (0.26 g, 1.3 mmol) to afford the desired product (0.15 g, 51% yield) as an off-white solid: ESI MS m/z 457 [C₂₁H₂₀N₄O₄S₂+H]⁺.

### Example 28

### N-[2-(4-Chlorophenylsulfonamido)ethyl]-7-methoxy-2-(thiophen-2-yl)-1H-benzo[d]imidazole-4-carboxamide

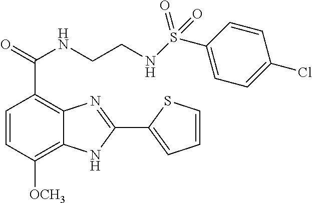

Following General Procedure B,

7-methoxy-2-(thiophen-2-yl)-1H-benzo[d]imidazole-4-carboxylic acid (0.20 g, 0.73 mmol) was reacted with N-(2-aminoethyl)-4-chlorobenzenesulfonamide (0.34 g, 1.5 mmol) to afford the desired product (0.16 g, 45% yield) as an off-white solid: ESI MS m/z 491 [C₂₁H₁₉ClN₄O₄S₂+H]⁺.

### Example 29

### 7-Methoxy-N-[2-(pyridine-4-sulfonamido)ethyl]-2-(thiophen-2-yl)-1H-benzo[d]imidazole-4-carboxamide

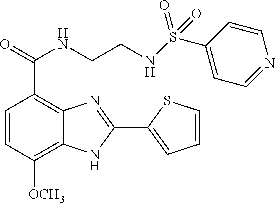

Following General Procedure B,

7-methoxy-2-(thiophen-2-yl)-1H-benzo[d]imidazole-4-carboxylic acid (0.20 g, 0.73 mmol) was reacted with N-(2-aminoethyl)pyridine-4-sulfonamide (0.29 g, 1.5 mmol) to afford the desired product (0.069 g, 21% yield) as an off-white solid: ESI MS m/z 458 [C₂₀H₁₉N₅O₄S₂+H]⁺.

### Example 30

### 7-Methoxy-N-[2-(4-methylbenzamido)ethyl]-2-(thiophen-2-yl)-1H-benzo[d]imidazole-4-carboxamide

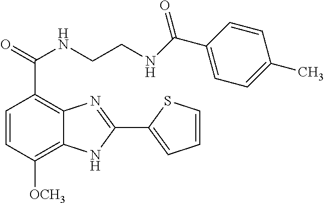

Following General Procedure B,

7-methoxy-2-(thiophen-2-yl)-1H-benzo[d]imidazole-4-carboxylic acid (0.20 g, 0.73 mmol) was reacted with N-(2-aminoethyl)-4-methylbenzamide (0.27 g, 1.5 mmol) to afford the desired product (0.24 g, 76% yield) as an off-white solid: ESI MS m/z 435 [C₂₃H₂₂N₄O₃S+H]⁺.

### Example 31

### N-(2-Acetamidoethyl)-7-methoxy-2-(thiophen-2-yl)-1H-benzo[d]imidazole-4-carboxamide

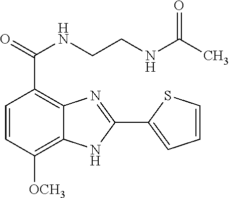

Following General Procedure B,

7-methoxy-2-(thiophen-2-yl)-1H-benzo[d]imidazole-4-carboxylic acid (0.10 g, 0.36 mmol) was reacted with N-(2-aminoethyl)acetamide (0.073 g, 0.72 mmol) to afford the crude desired product as an off-white solid: ESI MS m/z 356 [C₁₇H₁₈N₄O₃S+H]⁺.

### Example 32

### N-[3-(Isopropylamino)propyl]-7-methoxy-2-(thiophen-2-yl)-1H-benzo[d]imidazole-4-carboxamide

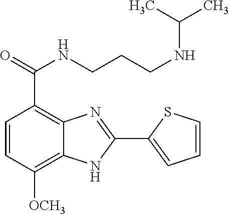

Following General Procedure B,

7-methoxy-2-(thiophen-2-yl)-1H-benzo[d]imidazole-4-carboxylic acid (0.20 g, 0.73 mmol) was reacted with N¹-isopropylpropane-1,3-diamine (0.17 g, 1.5 mmol) to afford the desired product as an off-white solid: ESI MS m/z 373 [C₁₉H₂₄N₄O₂S+H]⁺.

### Example 33

### N-(4-Fluorophenethyl)-7-methoxy-2-(thiophen-2-yl)-1H-benzo[d]imidazole-4-carboxamide

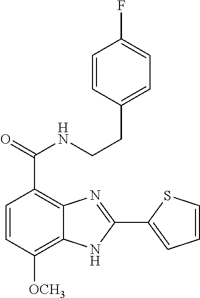

Following General Procedure B,

7-methoxy-2-(thiophen-2-yl)-1H-benzo[d]imidazole-4-carboxylic acid (0.20 g, 0.73 mmol) was reacted with 2-(4-fluorophenyl)ethanamine (0.21 g, 1.5 mmol) to afford the desired product (0.14 g) as an off-white solid: ESI MS m/z 396 [C₂₁H₁₈FN₃O₂S+H]⁺.

### Example 34

### N-(4-Hydroxyphenethyl)-7-methoxy-2-(thiophen-2-yl)-1H-benzo[d]imidazole-4-carboxamide

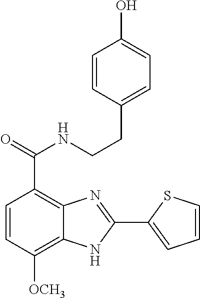

Following General Procedure B,

7-methoxy-2-(thiophen-2-yl)-1H-benzo[d]imidazole-4-carboxylic acid (0.20 g, 0.73 mmol) was reacted with 4-(2-aminoethyl)phenol (0.20 g, 1.5 mmol) to afford the desired product (0.31 g) as an off-white solid: ESI MS m/z 393 [C₂₁H₁₉N₃O₃S+H]⁺.

General Procedure C—synthesis of compounds of formula (I-III) as described in Scheme (1):

To a suspension of amides F (1.0 equiv) in dichloroethane (10-25 mL) was added boron tribromide (6.0-10 equiv) and the reaction mixture was heated at 80 degrees for 16 h. The reaction mixture was poured over ice and the resulting mixture was concentrated. The crude residue was eluted through an ion-exchange column (using methanol and 7 N methanol in ammonia) as a crude purification. The crude product was further purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to obtain the desired products.

### Example 35

### 7-Hydroxy-N-[2-(1-methyl-1H-imidazol-5-yl)ethyl]-2-(thiophen-2-yl)-1H-benzo[d]imidazole-4-carboxamide

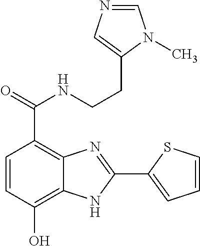

Following General Procedure C,

7-Methoxy-N-[2-(1-methyl-1H-imidazol-5-yl)ethyl]-2-(thiophen-2-yl)-1H-benzo[d]imidazole-4-carboxamide (170 mg) was reacted with boron tribromide to afford the desired product (36 mg, 16% yield) as a white solid: ¹H NMR (300 MHz, DMSO-d₆) delta 13.40 (s, 1H), 10.78 (s, 1H), 9.55 (s, 1H), 8.03 (s, 1H), 7.79-7.69 (m, 2H), 7.51 (s, 1H), 7.26-7.23 (m, 1H), 6.96 (s, 1H), 6.72 (d, J=8.1 Hz, 1H), 3.68-3.66 (m, 2H), 3.56 (s, 3H), 2.76 (t, J=6.9 Hz, 1H); ESI MS m/z 368 [C₁₈H₁₇N₅O₂S+H]⁺; HPLC>99% (AUC), t_{R}=10.67 min.

### Example 36

### N-[2-(dimethylamino)ethyl]-7-hydroxy-2-(thiophen-2-yl)-1H-benzo[d]imidazole-4-carboxamide

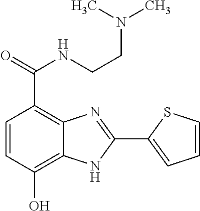

Following General Procedure C,

N-[2-(Dimethylamino)ethyl]-7-methoxy-2-(thiophen-2-yl)-1H-benzo[d]imidazole-4-carboxamide (136 mg) was reacted with boron tribromide to afford the desired product (69 mg, 35% yield) as a light yellow solid: ¹H NMR (300 MHz, CD₃OD) delta 7.88-7.87 (m, 1H), 7.76 (d, J=8.4 Hz, 1H), 7.64-7.62 (m, 1H), 7.22-7.19 (m, 1H), 6.68 (d, J=8.4 Hz, 1H), 3.69 (t, J=6.6 Hz, 2H), 2.75 (t, J=6.6 Hz, 1H), 2.43 (s, 6H); ESI MS m/z 331 [C₁₆H₁₈N₄O₂S+H]⁺; HPLC>99% (AUC), t_{R}=8.68 min.

### Example 37

### N-(3,4-Dihydroxybenzyl)-7-hydroxy-2-(thiophen-2-yl)-1H-benzo[d]imidazole-4-carboxamide

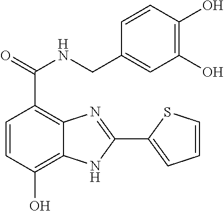

Following General Procedure C,

N-(3,4-Dimethoxybenzyl)-7-methoxy-2-(thiophen-2-yl)-1H-benzo[d]imidazole-4-carboxamide (248 mg) was reacted with boron tribromide to afford the desired product (18 mg, 8% yield) as a brown solid: ¹H NMR (300 MHz, CD₃OD) delta 7.98-7.97 (m, 1H), 7.82 (d, J=8.4 Hz, 1H), 7.77 (d, J=4.5 Hz, 1H), 7.28-7.25 (m, 1H), 6.90 (s, 1H), 6.83-6.77 (m, 3H), 4.55 (s, 2H); ESI MS m/z 382 [C₁₉H₁₅N₃O₄S+H]⁺; HPLC 97.0% (AUC), t_{R}=11.73 min.

### Example 38

### 7-Hydroxy-N-[2-(phenylsulfonamido)ethyl]-2-(thiophen-2-yl)-1H-benzo[d]imidazole-4-carboxamide

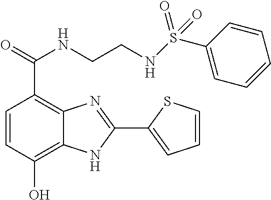

Following General Procedure C,

7-Methoxy-N-[2-(phenylsulfonamido)ethyl]-2-(thiophen-2-yl)-1H-benzo[d]imidazole-4-carboxamide (150 mg) was reacted with boron tribromide to afford the desired product (36 mg, 37% yield) as an off-white solid: ¹H NMR (500 MHz, CD₃OD) delta 7.91 (t, J=3.6 Hz, 1H), 7.82 (d, J=7.5 Hz, 2H), 7.74 (d, J=8.2 Hz, 1H), 7.64 (d, J=4.9 Hz, 1H), 7.40 (t, J=7.1 Hz, 1H), 7.34-7.31 (m, 2H), 7.21 (dd, J=5.0, 3.7 Hz, 1H), 6.69 (d, J=8.3 Hz, 1H), 3.60 (t, J=6.1 Hz, 2H), 3.19 (t, J=5.9 Hz, 2H); ESI MS m/z 443 [C₂₀H₁₈N₄O₄S₂+H]⁺; HPLC>99% (AUC), t_{R}=12.63 min.

### Example 39

### N-[2-(4-Chlorophenylsulfonamido)ethyl]-7-hydroxy-2-(thiophen-2-yl)-1H-benzo[d]imidazole-4-carboxamide

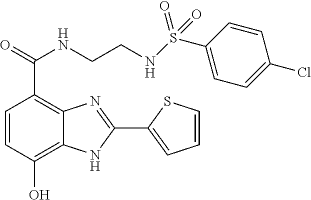

Following General Procedure C,

N-[2-(4-Chlorophenylsulfonamido)ethyl]-7-methoxy-2-(thiophen-2-yl)-1H-benzo[d]imidazole-4-carboxamide (160 mg) was reacted with boron tribromide to afford the desired product (17 mg, 18% yield) as an off-white solid: ¹H NMR (500 MHz, CD₃OD) delta 7.90 (d, J=0.9 Hz, 1H), 7.74-7.69 (m, 3H), 7.64 (d, J=4.8 Hz, 1H), 7.22 (t, J=3.9 Hz, 1H), 7.14 (d, J=8.4 Hz, 2H), 6.69 (d, J=8.4 Hz, 1H), 3.59-3.57 (m, 2H), 3.26-3.24 (m, 2H); ESI MS m/z 477 [C₂₀H₁₇ClN₄O₄S₂+H]⁺; HPLC>99% (AUC), t_{R}=13.39 min.

### Example 40

### 7-Hydroxy-N-[2-(pyridine-4-sulfonamido)ethyl]-2-(thiophen-2-yl)-1H-benzo[d]imidazole-4-carboxamide

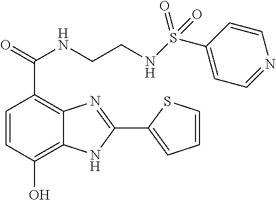

Following General Procedure C,

7-Methoxy-N-[2-(pyridine-4-sulfonamido)ethyl]-2-(thiophen-2-yl)-1H-benzo[d]imidazole-4-carboxamide (69 mg) was reacted with boron tribromide to afford the desired product (14 mg, 21% yield) as an off-white solid: ¹H NMR (500 MHz, CD₃OD) delta 8.93 (d, J=2.0 Hz, 1H), 8.52 (d, J=4.2 Hz, 1H), 8.19 (d, J=8.0 Hz, 1H), 8.02 (d, J=3.4 Hz, 1H), 7.80 (d, J=5.0 Hz, 1H), 7.72 (d, J=8.4 Hz, 1H), 7.39-7.35 (m, 1H), 7.29 (dd, J=4.9, 3.9 Hz, 1H), 6.78 (d, J=8.4 Hz, 1H), 3.58 (t, J=6.0 Hz, 2H), 3.27-3.25 (m, 2H); ESI MS m/z 444 [C₁₉H₁₇N₅O₄S₂+H]⁺; HPLC 98.5% (AUC), t_{R}=11.28 min.

### Example 41

### 7-Hydroxy-N-[2-(4-methylbenzamido)ethyl]-2-(thiophen-2-yl)-1H-benzo[d]imidazole-4-carboxamide

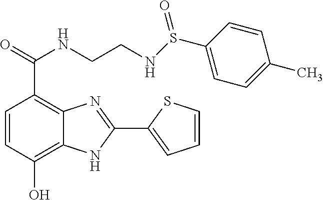

Following General Procedure C,

7-Methoxy-N-[2-(4-methylbenzamido)ethyl]-2-(thiophen-2-yl)-1H-benzo[d]imidazole-4-carboxamide (0.24 g) was reacted with boron tribromide to afford the desired product (165 mg, 71% yield) as an off-white solid: ¹H NMR (500 MHz, CD₃OD) delta 8.26 (dd, J=3.8, 1.0 Hz, 1H), 8.10 (dd, J=5.0, 1.0 Hz, 1H), 7.82 (d, J=8.4 Hz, 1H), 7.70 (d, J=8.2 Hz, 2H), 7.42 (dd, J=4.9, 3.9 Hz, 1H), 7.23 (d, J=8.0 Hz, 2H), 6.96 (d, J=8.4 Hz, 1H), 3.68 (s, 4H), 2.35 (s, 3H); ESI MS m/z 421 [C₁₉H₁₇N₅O₄S₂+H]⁺; HPLC 95.8% (AUC), t_{R}=12.69 min.

### Example 42

### N-(2-Acetamidoethyl)-7-hydroxy-2-(thiophen-2-yl)-1H-benzo[d]imidazole-4-carboxamide

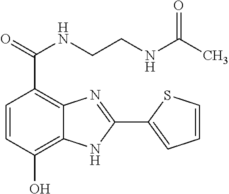

Following General Procedure C,

N-(2-Acetamidoethyl)-7-methoxy-2-(thiophen-2-yl)-1H-benzo[d]imidazole-4-carboxamide (73 mg) was reacted with boron tribromide to afford the desired product (28 mg, 25% yield) as a light brown solid: ¹H NMR (500 MHz, CD₃OD) 7.88 (d, J=3.5 Hz, 1H), 7.79 (d, J=8.5 Hz, 1H), 7.63 (d, J=5.0 Hz, 1H), 7.20 (t, J=4.5 Hz, 1H), 6.70 (d, J=8.0 Hz, 1H), 3.67 (t, J=6.0 Hz, 2H), 3.47 (t, J=6.0 Hz, 2H), 1.96 (s, 3H); ESI MS m/z 345 [C₁₆H₁₆N₄O₃S+H]⁺.

### Example 43

### N-[3-(Isopropylamino)propyl]-7-hydroxy-2-(thiophen-2-yl)-1H-benzo[d]imidazole-4-carboxamide

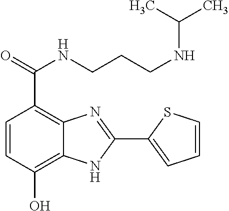

Following General Procedure C,

N-[3-(Isopropylamino)propyl]-7-methoxy-2-(thiophen-2-yl)-1H-benzo[d]imidazole-4-carboxamide (170 mg) was reacted with boron tribromide to afford the desired product (32 mg, 12% yield) as a light brown solid: ¹H NMR (500 MHz, DMSO-d₆) 9.50 (s, 1H), 8.06 (d, J=2.5 Hz, 1H), 7.76 (d, J=4.5 Hz, 1H), 7.66 (d, J=8.5 Hz, 1H), 7.23 (dd, J=5.0, 4.0 Hz, 1H), 6.68 (d, J=8.0 Hz, 1H), 3.46 (t, J=6.0 Hz, 2H), 2.81-2.78 (m, 1H), 2.72 (t, J=7.0 Hz, 2H), 1.75-1.72 (m, 6H); ESI MS m/z 359 [C₁₇H₁₈N₄O₃S+H]⁺; HPLC 98.2% (AUC), t_{R}=8.30 min.

### Example 44

### N-(4-Fluorophenethyl)-7-hydroxy-2-(thiophen-2-yl)-1H-benzo[d]imidazole-4-carboxamide

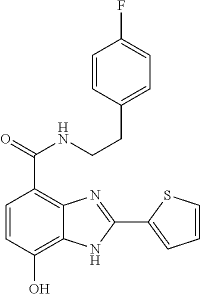

Following General Procedure C,

N-(4-Fluorophenethyl)-7-methoxy-2-(thiophen-2-yl)-1H-benzo[d]imidazole-4-carboxamide (140 mg) was reacted with boron tribromide to afford the desired product (17 mg, 13% yield) as a light brown solid: ¹H NMR (500 MHz, DMSO-d₆) 7.67 (bs, 1H), 7.41-7.36 (m, 4H), 7.10-7.07 (m, 3H), 6.17 (d, J=7.5 Hz, 1H), 3.63-3.60 (m, 2H), 2.90 (t, J=7.0 Hz, 2H); ESI MS m/z 382 [C₂₀H₁₆FN₃O₂S+H]⁺; HPLC 92.4% (AUC), t_{R}=14.48 min.

### Example 45

### 7-Hydroxy-N-(4-hydroxyphenethyl)-2-(thiophen-2-yl)-1H-benzo[d]imidazole-4-carboxamide

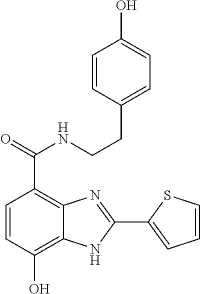

Following General Procedure C,

N-(4-Hydroxyphenethyl)-7-methoxy-2-(thiophen-2-yl)-1H-benzo[d]imidazole-4-carboxamide (310 mg) was reacted with boron tribromide to afford the desired product (19 mg, 7% yield) as a brown solid: ¹H NMR (500 MHz, CD₃OD) 7.79-7.74 (m, 2H), 7.61-7.60 (m, 1H), 7.19-7.15 (m, 3H), 6.71-6.65 (m, 3H), 3.74-3.71 (m, 2H), 2.92-2.89 (m, 2H); ESI MS m/z 380 [C₂₀H₁₇N₃O₃S+H]⁺; HPLC>99% (AUC), t_{R}=12.54 min.

### Example 46

### N-(2-Hydroxyethyl)-7-methoxy-2-(thiophen-2-yl)-1H-benzo[d]imidazole-4-carboxamide

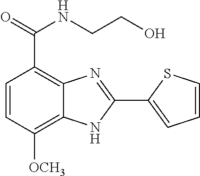

A suspension of methyl 7-methoxy-2-(thiophen-2-yl)-1H-benzo[d]imidazole-4-carboxylate (970 mg, 3.36 mmol) in ethanolamine (5 mL) was heated at 100 degrees for 18 h. The reaction mixture was cooled and diluted with water (50 mL). The resulting precipitate was filtered and washed with water to afford the desired product (850 mg, 80% yield) as a brown solid: ESI MS m/z 318 [C₁₅H₁₅N₃O₃S+H]⁺.

### Example 47

### N-[2-(5-Carbamoylpyridin-2-yloxy)ethyl]-7-methoxy-2-(thiophen-2-yl)-1H-benzo[d]imidazole-4-carboxamide

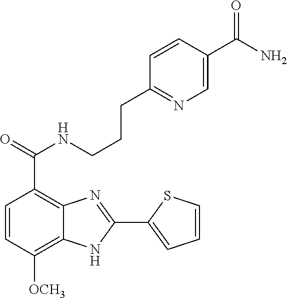

To a solution of 7-methoxy-2-(thiophen-2-yl)-1H-benzo[d]imidazole-4-carboxylate (100 mg, 0.31 mmol) in DMF (5 mL) was added NaH (60 mg, 1.5 mmol, 60% dispersion) and the suspension was stirred at room temperature for 1 h. Following the addition of 6-chloro-nicotinamide (74 mg, 0.47 mmol), the reaction mixture was heated at 85 degrees for 18 h. The reaction mixture was cooled and quenched with water (20 mL) and the pH was adjusted to 7. The resulting precipitate was filtered and washed with water to afford the desired product (105 mg, crude) as a brown solid: ESI MS m/z 438 [C₂₁H₁₉N₅O₄S+H]⁺.

### Example 48

### 7-methoxy-2-(thiophen-2-yl)-N-(2-(5-(trifluoromethyl)pyridin-2-yloxy)ethyl)-1H-benzo[d]imidazole-4-carboxamide

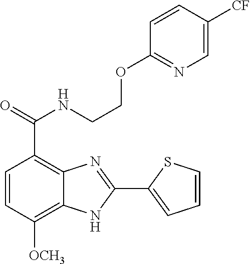

To a solution of 7-methoxy-2-(thiophen-2-yl)-1H-benzo[d]imidazole-4-carboxylate (125 mg, 0.39 mmol) in DMF (5 mL) was added NaH (75 mg, 1.95 mmol, 60% dispersion) and the suspension was stirred at room temperature for 1 h. Following the addition of 2-chloro-5-trifluoromethyl-pyridine (143 mg, 0.78 mmol), the reaction mixture was heated at 85 degrees for 18 h. The reaction mixture was cooled and quenched with water (20 mL) and the pH was adjusted to 7. The resulting precipitate was filtered and washed with water to afford the desired product (180 mg, crude) as a brown solid: ESI MS m/z 463 [C₂₁H₁₇F₃N₄O₃S+H]⁺.

### Example 49

### N-[2-(5-Carbamoylpyridin-2-yloxy)ethyl]-7-hydroxy-2-(thiophen-2-yl)-1H-benzo[d]imidazole-4-carboxamide

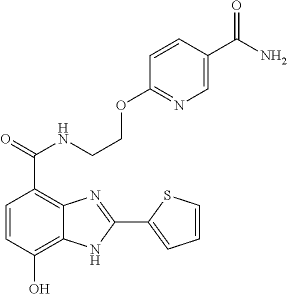

Following General Procedure C,

N-(2-(5-carbamoylpyridin-2-yloxy)ethyl)-7-methoxy-2-(thiophen-2-yl)-1H-benzo[d]imidazole-4-carboxamide (0.24 mmol) was reacted with boron tribromide to afford the desired product (28 mg, 28% yield) as a light yellow solid: ¹H NMR (300 MHz, CD₃OD) delta 8.69-8.68 (m, 1H), 8.12-8.09 (m, 1H), 7.74-7.69 (m, 2H), 7.49 (d, J=5.1 Hz, 1H), 7.15-7.13 (m, 1H), 6.98 (d, J=8.7 Hz, 1H), 6.48 (d, J=8.4 Hz, 1H), 4.64 (t, J=5.1 Hz, 2H), 3.93 (t, J=5.1 Hz, 2H); ESI MS m/z 424 [C₂₀H₁₇N₅O₄S+H]⁺; HPLC 98.9% (AUC), t_{R}=11.01 min.

### Example 50

### 7-Hydroxy-2-(thiophen-2-yl)-N-[2-(5-(trifluoromethyl)pyridin-2-yloxy)ethyl]-1H-benzo[d]imidazole-4-carboxamide

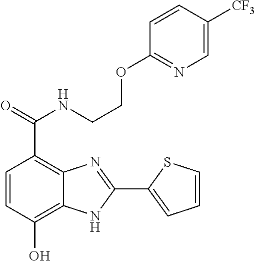

Following General Procedure C,

7-methoxy-2-(thiophen-2-yl)-N-(2-(5-(trifluoromethyl)pyridin-2-yloxy)ethyl)-1H-benzo[d]imidazole-4-carboxamide (0.39 mmol) was reacted with boron tribromide to obtain the desired product (33 mg, 19% yield over 2 steps) as a white solid: ¹H NMR (300 MHz, CD₃OD) delta 8.42 (s, 1H), 7.96-7.87 (m, 2H), 7.80-7.72 (m, 2H), 7.26-7.23 (m, 1H), 7.01 (d, J=9.0 Hz, 1H), 6.77 (d, J=8.4 Hz, 1H), 4.67 (t, J=5.1 Hz, 2H), 3.92 (t, J=5.1 Hz, 2H); ESI MS m/z 449 [C₂₀H₁₅F₃N₄O₃S+H]⁺; HPLC>99% (AUC), t_{R}=14.71 min.

### Example 51

### Methyl 3-(1-tosyl-1H-imidazol-2-yl)acrylate

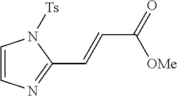

To a suspension of 1-tosyl-1H-imidazole-2-carbaldehyde (1.54 g, 6.2 mmol) in THF (75 mL) was added methyl(triphenylphosphoranylidene) acetate (2.46 g, 7.4 mmol) and the reaction mixture was heated at 75 degrees for 18 h. The reaction mixture was cooled, diluted with satd. aq NaHCO₃, extracted with ethyl acetate (100 mL), dried over Na₂SO₄, and purified by column chromatography (silica, 0-50% ethyl acetate/heptane) to afford the desired product (1.43 g, 76% yield) as a clear oil: ESI MS m/z 307 [C₁₄H₁₄N₂O₄S+H]⁺.

### Example 52

### Methyl 3-(1-tosyl-1H-imidazol-2-yl)propanoate

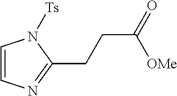

To a solution of methyl 3-(1-tosyl-1H-imidazol-2-yl)acrylate (1.43 g, 4.67 mmol) in MeOH (50 mL) was added cat. 10 wt % Pd/C (200 mg) and the reaction mixture was stirred under an atmosphere of hydrogen gas (1 atm) at room temperature for 18 h. The reaction mixture was filtered through diatomaceous earth, washed with MeOH, and concentrated to afford the desired product (1.35 g, 94% yield) as a waxy solid: ESI MS m/z 309 [C₁₄H₁₆N₂O₄S+H]⁺.

### Example 53

### Synthesis of 3-(1-Tosyl-1H-imidazol-2-yl)propan-1-ol

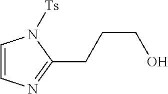

To a solution of methyl 3-(1-tosyl-1H-imidazol-2-yl)propanoate (1.35 g, 4.39 mmol) in THF (50 mL) at 0 degree was added DIBAL (11.8 mL, 11.8 mmol, 1.0 M) and the reaction mixture was stirred for 1.5 h. The reaction mixture was warmed to room temperature over 2 h, concentrated, and purified by column chromatography (silica gel, 0-75% ethyl acetate/heptane) to afford the desired product (492 mg, 40% yield) as a white solid: ESI MS m/z 281 [C₁₃H₁₆N₂O₃S+H]⁺.

### Example 54

### 2-[3-(1-Tosyl-1H-imidazol-2-yl)propyl]isoindoline-1,3-dione

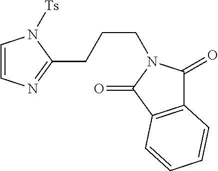

A solution of 3-(1-Tosyl-1H-imidazol-2-yl)propan-1-ol (492 mg, 1.75 mmol), triphenylphosphine (636 mg, 2.63 mmol), and phthalimide (386 mg, 2.63 mmol) in THF (20 mL) was cooled to 0 degree and diisopropyl azodicarboxylate (532 mg, 2.63 mmol) was added and the reaction mixture was stirred at room temperature for 18 h. The reaction mixture was diluted with ethyl acetate (75 mL), washed with water (30 mL) and brine (30 mL), dried over Na₂SO₄, and purified by column chromatography (silica gel, 0-75% ethyl acetate/heptane) to afford the desired product (698 mg, 97% yield) as a white foam: ESI MS m/z 410 [C₂₁H₁₉N₃O₄S+H]⁺.

### Example 55

### 3-(1H-Imidazol-2-yl)propan-1-amine

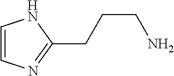

To a suspension of 2-[3-(1-tosyl-1H-imidazol-2-yl)propyl]isoindoline-1,3-dione (698 mg, 1.70 mmol) in EtOH (25 mL) was added hydrazine hydrate (1.9 mL, 34 mmol) and the reaction mixture was heated at reflux for 3 h. The reaction mixture was cooled and the resulting solids were filtered and washed with EtOH. The filtrate was concentrated and the crude product was eluted through an ion-exchange column (using methanol and 7 N methanol in ammonia) to afford the desired product (330 mg, crude) as a clear oil: ¹H NMR (300 MHz, CD₃OD) delta 6.90 (s, 2H), 2.82-2.65 (m, 4H), 1.86 (p, J=7.2 Hz, 2H).

### Example 56

### N-[3-(1H-Imidazol-2-yl)propyl]-7-methoxy-2-(thiophen-2-yl)-1H-benzo[d]imidazole-4-carboxamide

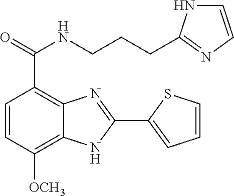

Following General Procedure B,

7-methoxy-2-(thiophen-2-yl)-1H-benzo[d]imidazole-4-carboxylic acid (0.15 g, 0.56 mmol) was reacted with 3-(1H-Imidazol-2-yl)propan-1-amine (0.14 g, 1.2 mmol) to afford the desired product (56 mg crude) as a tan solid: ESI MS m/z 382 [C₁₉H₁₉N₅O₂S+H]⁺.

### Example 57

### N-3-(1H-Imidazol-2-yl)propyl)-]7-hydroxy-2-(thiophen-2-yl)-1H-benzo[d]imidazole-4-carboxamide

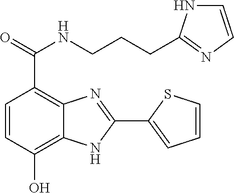

Following General Procedure C,

N-[3-(1H-Imidazol-2-yl)propyl]-7-methoxy-2-(thiophen-2-yl)-1H-benzo[d]imidazole-4-carboxamide (0.15 mmol) was reacted with boron tribromide to afford the desired product (20 mg, 37% yield) as a light brown solid: ¹H NMR (300 MHz, DMSO-d₆) delta 13.45 (s, 1H), 11.86 (s, 1H), 10.81 (s, 1H), 9.63 (s, 1H), 8.07 (s, 1H), 7.77-7.68 (m, 2H), 7.25-7.22 (m, 1H), 6.88 (s, 2H), 6.73 (d, J=8.1 Hz, 1H), 3.47-3.45 (m, 2H), 2.82-2.73 (m, 2H), 1.97 (p, J=7.2 Hz, 2H); ESI MS m/z 368 [C₁₈H₁₇N₅O₂S+H]⁺; HPLC>99% (AUC), t_{R}=9.21 min.

### Example 58

### tert-Butyl 3-oxopropylcarbamate

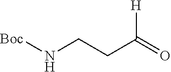

To a solution of tert-butyl 3-hydroxypropylcarbamate (0.50 g, 2.8 mmol) in CH₂Cl₂ (30 mL) was added Dess-Martin periodinane (1.3 g, 3.1 mmol) and pyridine (450 mg, 5.7 mmol) and the reaction mixture was stirred at room temperature for 18 h. The reaction mixture was quenched by the addition of satd. aq NaHCO₃ (20 mL) and solid Na₂S₂O₃ (1.0 g). The layers were separated and the aqueous layer was extracted with diethyl ether (25 mL). The combined organic layers were dried over Na₂SO₄, concentrated, and purified by column chromatography (silica gel, 0-75% ethyl acetate/heptane) to afford the desired product (360 mg, 73% yield) as an oil: ¹H NMR (300 MHz, CDCl₃) delta 9.83 (s, 1H), 4.91 (s, 1H), 3.44 (q, J=5.9 Hz, 2H), 2.73 (t, J=5.9 Hz, 2H), 1.45 (s, 9H).

### Example 59

### tert-Butyl 2-(4,5-dihydro-1H-imidazol-2-yl)ethylcarbamate

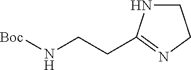

To a solution of tert-Butyl 3-oxopropylcarbamate (360 mg, 2.09 mmol) in t-BuOH (20 mL) was added ethylenediamine (138 mg, 2.3 mmol) and the reaction mixture was stirred at room temperature for 18 h. Potassium carbonate (867 mg, 6.27 mmol) and iodine (690 mg, 2.72 mmol) were added and the reaction mixture was heated at 70 degrees for 2 h. The reaction mixture was quenched by the addition of satd. aq Na₂S₂O₃ (20 mL) and the pH was adjusted to 12 with 1 M NaOH. The reaction mixture was extracted with 3:1 CHCl₃/IPA (50 mL) and concentrated to afford the desired product (370 mg, 83% yield) as an orange oil: ESI MS m/z 214 [C₁₀H₁₉N₃O₂+H]⁺.

### Example 60

### 2-(1H-imidazol-2-yl)ethanamine

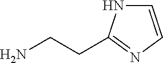

To a solution of tent-Butyl 2-(4,5-dihydro-1H-imidazol-2-yl)ethylcarbamate (370 mg, 1.73 mmol) in DMSO (5 mL) was added potassium carbonate (528 mg, 3.82 mmol) and iodobenzene diacetate (1.23 g, 3.82 mmol) and the reaction mixture was stirred at room temperature for 18 h. The reaction mixture was heated at 50 degrees for 3 h, cooled, diluted with water (25 mL) and extracted with 3:1 CHCl₃/i-propanol (50 mL). The organic layer was dried over Na₂SO₄, concentrated, and the crude product was dissolved in CH₂Cl₂ (10 mL) followed by the addition of trifluoroacetic acid (2 mL) and the reaction mixture was stirred at room temperature for 18 h. The reaction mixture was concentrated and the crude residue was eluted through an ion-exchange column (using methanol and 7 N methanol in ammonia) to afford the desired product (140 mg) as a brown solid: ¹H NMR (300 MHz, CD₃OD) delta 6.95 (s, 2H), 3.04-2.91 (m, 2H), 2.87-2.76 (m, 2H).

### Example 61

### N-[2-(1H-Imidazol-2-yl)ethyl]-7-methoxy-2-(thiophen-2-yl)-1H-benzo[d]imidazole-4-carboxamide

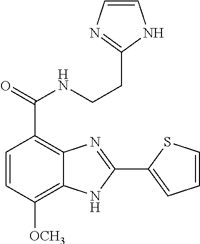

To a suspension of 7-methoxy-2-(thiophen-2-yl)-1H-benzo[d]imidazole-4-carboxylic acid (0.34 g, 1.3 mmol) in toluene (15 mL) was added thionyl chloride (0.61 g, 5.2 mmol). After stirring at room temperature for 16 h, the reaction mixture was heated at 70 degrees for 2 h. The reaction mixture was cooled to room temperature and concentrated. The residue was suspended in THF (20 mL) followed by the addition of pyridine (98 mg, 2.6 mmol) and 2-(1H-imidazol-2-yl)ethanamine (140 mg) and the reaction mixture was heated at 70 degrees for 16 h. The reaction mixture was concentrated and the residue was diluted with water (20 mL) and extracted with ethyl acetate (3×20 mL). The combined organic layers were washed with satd. aq NaHCO₃ (20 mL), concentrated, and purified by flash chromatography (silica, 0-15% methanol/dichloromethane) to afford the desired product: ESI MS m/z 368 [C₁₈H₁₇N₅O₂S+H]⁺.

### Example 62

### N-[2-(1H-Imidazol-2-yl)ethyl]-7-hydroxy-2-(thiophen-2-yl)-1H-benzo[d]imidazole-4-carboxamide

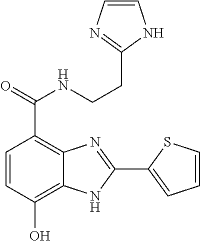

Following General Procedure C,

N-(2-(1H-imidazol-2-yl)ethyl)-7-methoxy-2-(thiophen-2-yl)-1H-benzo[d]imidazole-4-carboxamide from Example 59 was reacted with boron tribromide to afford the desired product (5 mg, 3% yield) as a light brown solid: ¹H NMR (300 MHz, CD₃OD) delta 7.85-7.84 (m, 1H), 7.74 (d, J=8.4 Hz, 1H), 7.63 (d, J=5.1 Hz, 1H), 7.21-7.18 (m, 1H), 7.06 (s, 2H), 6.69 (d, J=8.4 Hz, 1H), 3.90 (t, J=6.6 Hz, 2H), 3.15 (d, J=6.6 Hz, 1H); ESI MS m/z 354 [C₁₇H₁₅N₅O₂S+H]⁺; HPLC 97.7% (AUC), t_{R}=9.56 min.

### Example 63

### 7-Methoxy-2-(thiophen-2-yl)-1H-benzo[d]imidazole-5-carboxamide

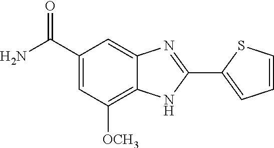

Following General Procedure B,

7-methoxy-2-(thiophen-2-yl)-1H-benzo[d]imidazole-5-carboxylic acid (150 mg, 0.55 mmol) was reacted with excess NH₄OH to afford the desired product (42 mg) as a brown solid: ESI MS m/z 274 [C₁₃H₁₁N₃O₂S+H]⁺.

### Example 64

### N-[2-(1H-Imidazol-5-yl)ethyl]-7-methoxy-2-(thiophen-2-yl)-1H-benzo[d]imidazole-5-carboxamide

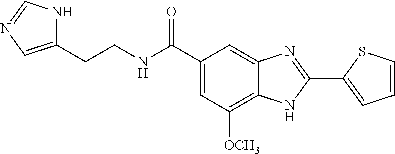

Following General Procedure B,

7-methoxy-2-(thiophen-2-yl)-1H-benzo[d]imidazole-5-carboxylic acid (150 mg, 0.55 mmol) was reacted with histamine (0.14 g, 1.1 mmol) to afford the desired product (92 mg) as a brown solid: ESI MS m/z 368 [C₁₈H₁₇N₅O₂S+H]⁺.

### Example 65

### 7-Hydroxy-2-(thiophen-2-yl)-1H-benzo[d]imidazole-5-carboxamide

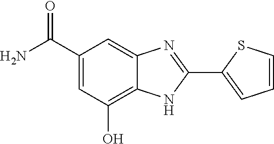

Following General Procedure C,

7-methoxy-2-(thiophen-2-yl)-1H-benzo[d]imidazole-5-carboxamide (40 mg) was reacted with boron tribromide to afford the desired product (13 mg, 32% yield) as an off-white solid: ¹H NMR (500 MHz, CD₃OD) delta 7.83-7.82 (m, 1H), 7.65-7.63 (m, 1H), 7.61 (s, 1H), 7.22-7.20 (m, 1H), 7.18 (s, 1H); ESI MS m/z 260 [C₁₂H₉N₃O₂S+H]⁺; HPLC>99% (AUC), t_{R}=9.32 min.

### Example 66

### N-[2-(1H-Imidazol-5-yl)ethyl]-7-hydroxy-2-(thiophen-2-yl)-1H-benzo[d]imidazole-5-carboxamide

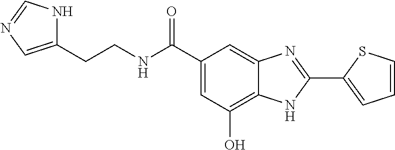

Following General Procedure C,

N-[2-(1H-imidazol-5-yl)ethyl]-7-methoxy-2-(thiophen-2-yl)-1H-benzo[d]imidazole-5-carboxamide was reacted with boron tribromide to afford the desired product (12 mg, 14% yield) as a light yellow solid: ¹H NMR (500 MHz, CD₃OD) delta 7.82 (s, 1H), 7.69 (s, 1H), 7.64-7.63 (m, 1H), 7.53 (s, 1H), 7.21-7.20 (m, 1H), 7.10 (s, 1H), 6.93 (s, 1H), 3.64 (t, J=7.0 Hz, 2H), 2.93 (t, J=7.0 Hz, 2H); ESI MS m/z 354 [C₁₇H₁₅N₅O₂S+H]⁺; HPLC 98.9% (AUC), t_{R}=7.57 min.

### Example 67

### 7-Hydroxy-N-[2-(5-nitropyridin-2-ylamino)ethyl]-2-(thiophen-2-yl)-1H-benzo[d]imidazole-4-carboxamide

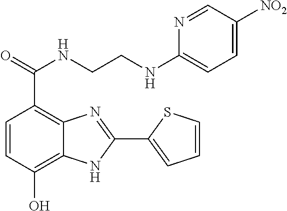

To a suspension of 7-hydroxy-2-(thiophen-2-yl)-1H-benzo[d]imidazole-4-carboxylic acid (0.65 g, 2.5 mmol) in dichloromethane (25 mL) was added N¹-(5-nitropyridin-2-yl)ethane-1,2-diamine (0.50 g, 2.75 mmol), EDC (0.58 g, 3.0 mmol), HOBt (0.40 g, 3.0 mmol), and DIPEA (0.97 g, 7.5 mmol) and the reaction mixture was stirred at room temperature for 16 h. Analysis by LC-MS indicated that the reaction was not complete, therefore, the dichloromethane was removed under reduced pressure, the residue was dissolved in DMF (5 mL), and the reaction mixture was heated at 50 degrees for 16 h. The reaction mixture was cooled, concentrated under reduced pressure, and triturated with water (20 mL) to afford the desired product (0.45 g, 42% yield) as a yellow-brown solid: ESI MS m/z 425 [C₁₉H₁₆N₆O₄S+H]⁺. This intermediate was used without further purification or characterization.

### Example 68

### N-[2-(5-Aminopyridin-2-ylamino)ethyl]-7-hydroxy-2-(thiophen-2-yl)-1H-benzo[d]imidazole-4-carboxamide Hydrochloride

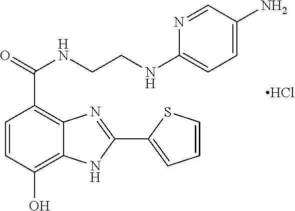

To a solution of

7-hydroxy-N-(2-(5-nitropyridin-2-ylamino)ethyl)-2-(thiophen-2-yl)-1H-benzo[d]imidazole-4-carboxamide (0.22 g, 0.52 mmol) in ethanol (5 mL) and 6 N HCl (5 mL) was added iron powder (0.12 g, 2.1 mmol) and the reaction mixture was heated at reflux for 4 h. The reaction mixture was cooled to room temperature and concentrated to provide the desired product which was immediately carried forward without further purification or characterization: ESI MS m/z 395 [C₁₉H₁₈N₆O₂S+H]⁺.

### Example 69

### 7-Hydroxy-N-{2-[5-(methylsulfonamido)pyridin-2-ylamino]ethyl}-2-(thiophen-2-yl)-1H-benzo[d]imidazole-4-carboxamide

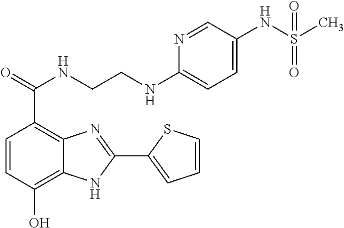

To a solution of crude

N-(2-(5-aminopyridin-2-ylamino)ethyl)-7-hydroxy-2-(thiophen-2-yl)-1H-benzo[d]imidazole-4-carboxamide hydrochloride (0.29 g, 0.68 mmol) in DMF (5 mL) was added DIPEA (0.44 g, 3.4 mmol) and methanesulfonyl chloride (0.085 g, 0.75 mmol) and the reaction mixture was stirred for 16 h at room temperature. The reaction mixture was concentrated under reduced pressure and purified by flash chromatography (silica, 0-20% methanol/dichloromethane) to afford the desired product (59 mg, 18% yield) as a white solid: ¹H NMR (500 MHz, CD₃OD) delta 7.87 (d, J=2.5 Hz, 1H), 7.82-7.78 (m, 2H), 7.59 (d, J=4.5 Hz, 1H), 7.37 (dd, J=8.5, 2.5 Hz, 1H), 7.17 (s, 1H), 6.69 (d, J=8.5 Hz, 1H), 6.62 (d, J=8.5 Hz, 1H), 3.78 (bs, 2H), 3.64 (bs, 2H), 2.83 (s, 3H); ESI MS m/z 473 [C₂₀H₂₀N₆O₄S₂+H]⁺; HPLC>99% (AUC), t_{R}=10.42 min.

### Example 70

### N-[2-(5-Acetamidopyridin-2-ylamino)ethyl]-7-hydroxy-2-(thiophen-2-yl)-1H-benzo[d]imidazole-4-carboxamide

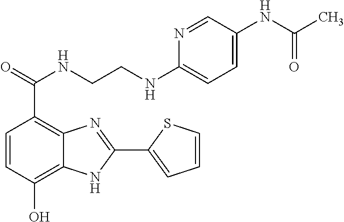

To a solution of

N-(2-(5-aminopyridin-2-ylamino)ethyl)-7-hydroxy-2-(thiophen-2-yl)-1H-benzo[d]imidazole-4-carboxamide hydrochloride (0.22 g, 0.52 mmol) in DMF (5 mL) was added DIPEA (0.34 g, 2.6 mmol) and acetyl chloride (0.045 g, 0.57 mmol) and the reaction mixture was stirred for 16 h at room temperature. The reaction mixture was concentrated under reduced pressure and purified by flash chromatography (silica, 0-20% methanol/dichloromethane) to afford the desired product (55 mg, 24% yield) as an off-white solid: ¹H NMR (500 MHz, CD₃OD) delta 8.42 (s, 1H), 7.85 (d, J=3.5 Hz, 1H), 7.80 (d, J=6.5 Hz, 1H), 7.69 (dd, J=9.5, 2.0 Hz, 1H), 7.61 (d, J=4.5 Hz, 1H), 7.21 (d, J=4.0 Hz, 1H), 7.07 (d, J=9.0 Hz, 1H), 6.71 (d, J=8.5 Hz, 1H), 3.82-3.81 (m, 2H), 3.71-3.68 (m, 2H) 2.12 (s, 3H); ESI MS m/z 437 [C₂₁H₂₀N₆O₃S+H]⁺; HPLC 96.0% (AUC), t_{R}=9.97 min.

### Example 71

### (S)-2-[7-Hydroxy-2-(thiophen-2-yl)-1H-benzo[d]imidazole-4-carboxamido]-3-(1H-imidazol-5-yl)propanoic Acid

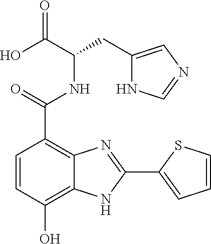

A solution of (S)-methyl

2-(7-hydroxy-2-(thiophen-2-yl)-1H-benzo[d]imidazole-4-carboxamido)-3-(1H-imidazol-5-yl) propanoate (30 mg, 0.062 mmol) in 3 M NaOH (10 ml) was heated at 80 degrees for 4 h. The reaction mixture was cooled to room temperature and acidified to pH 5 with 3 M HCl. The resulting precipitate was filtered and the crude solid was purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to afford the desired product (9.1 mg, 31% yield) as a yellow solid: ¹H NMR (500 MHz, CD₃OD) delta 8.22 (s, 1H), 7.86 (d, J=2.9 Hz, 1H), 7.70 (d, J=8.2 Hz, 1H), 7.58 (d, J=4.7 Hz, 1H), 7.22 (s, 1H), 7.15 (t, J=4.2 Hz, 1H), 6.66 (d, J=8.3 Hz, 1H), 3.38-3.33 (m, 2H); ESI MS m/z 398 [C₁₈H₁₅N₅O₄S+H]⁺; HPLC 97.8% (AUC), t_{R}=7.07 min.

### Example 72

### (S)-2-[7-Hydroxy-2-(thiophen-2-yl)-1H-benzo[d]imidazole-4-carboxamido]-3-(1H-indol-3-yl)propanoic Acid

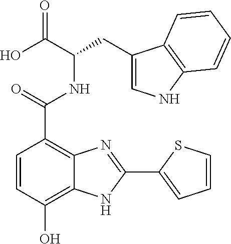

A solution of (S)-methyl 2-(7-hydroxy-2-(thiophen-2-yl)-1H-benzo[d]imidazole-4-carboxamido)-3-(1H-indol-3-yl) propanoate (40 mg, 0.060 mmol) in 3 M NaOH (10 ml) was heated at 80 degrees for 4 h. The reaction mixture was cooled to room temperature and acidified to pH 5 with 3 M HCl. The resulting precipitate was filtered and the crude solid was purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to afford the desired product (25 mg, 64% yield) as a yellow solid: ¹H NMR (500 MHz, CD₃OD) delta 7.76 (bs, 1H), 7.63 (bs, 2H), 7.49 (bs, 1H), 7.29 (bs, 1H), 7.21 (d, J=4.2 Hz, 1H), 7.10 (bs, 1H), 6.99 (t, J=7.5 Hz, 1H), 6.89 (d, J=3.2 Hz, 1H), 6.56 (bs, 1H), 3.51 (bs, 1H), 3.51-3.38 (m, 1H); ESI MS m/z 447 [C₂₃H₁₈N₄O₄S+H]⁺; HPLC 97.8% (AUC), t_{R}=7.07 min.

### Example 73

### 7-Hydroxy-N-[2-(4-nitrophenoxy)ethyl]-2-(thiophen-2-yl)-1H-benzo[d]imidazole-4-carboxamide

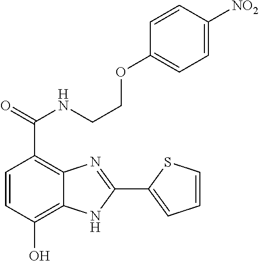

Following General Procedure A,

7-hydroxy-2-(thiophen-2-yl)-1H-benzo[d]imidazole-4-carboxylic acid (0.25 g, 0.95 mmol) was reacted with 2-(4-nitrophenoxy)ethanamine (0.34 g, 1.9 mmol) to obtain the desired product (230 mg, 57%) as a yellow solid: ¹H NMR (500 MHz, DMSO-d₆) delta 13.44 (s, 1H), 10.83 (s, 1H), 9.85 (s, 1H), 8.23-8.20 (m, 2H), 8.03-8.02 (m, 1H), 7.75-7.71 (m, 2H), 7.28-7.21 (m, 3H), 6.73 (d, J=8.3 Hz, 1H), 4.35 (t, J=10.0 Hz, 2H), 3.88 (t, J=11.0 Hz, 2H); ESI MS m/z 425 [C₂₀H₁₆N₄O₅S+H]⁺; HPLC 98.2% (AUC), t_{R}=13.25 min.

### Example 74

### 7-Hydroxy-N-{2-[4-(4-methylphenylsulfonamido)phenoxy]ethyl}-2-(thiophen-2-yl)-1H-benzo[d]imidazole-4-carboxamide

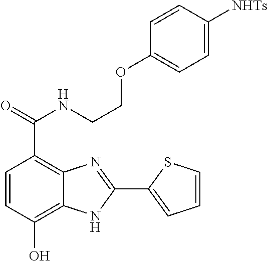

To a solution of

7-hydroxy-N-(2-(4-nitrophenoxy)ethyl)-2-(thiophen-2-yl)-1H-benzo[d]imidazole-4-carboxamide (0.20 g, 0.48 mmol) in EtOH (20 mL) was added iron filings (160 mg, 2.8 mmol) and 6 N HCl (15 mL, 90 mmol) and the reaction mixture was heated at reflux for 16 h. The reaction mixture was cooled to room temperature and concentrated. The resulting crude aniline was dissolved in DMF (5 mL) followed by the addition of p-toluenesulfonyl chloride (0.13 g, 0.72 mmol) and DIPEA (0.16 g, 1.3 mmol). The reaction mixture was stirred at room temperature for 16 h, quenched with satd. aq NaCl (50 mL), and extracted with ethyl acetate (2×50 mL). The combined organic layers were washed with satd. aq NaCl (50 mL), dried over Na₂SO₄, concentrated, and purified by preparatory HPLC (C18 silica, 10-90% acetonitrile/water with 0.05% TFA) to afford the desired product (15 mg, 6% yield) as a light yellow solid: ¹H NMR (300 MHz, CD₃OD) delta 7.81-7.76 (m, 2H), 7.54-7.51 (m, 3H), 7.21 (d, J=8.0 Hz, 2H), 7.13 (t, J=8.3 Hz, 1H), 6.92 (q, J=8.8 Hz, 4H), 6.69 (d, J=8.3 Hz, 1H), 4.18 (t, J=5.0 Hz, 2H), 3.86 (t, J=5.1 Hz, 2H), 2.32 (s, 3H); ESI MS m/z 549 [C₂₇H₂₄N₄O₅S₂+H]⁺; HPLC>99% (AUC), t_{R}=14.76 min.

Wherein, NHTs means p-toluenesulfonamido.

### Example 75

### N-[3-(1H-Imidazol-1-yl)propyl]-7-methoxy-2-(thiophen-2-yl)-1H-benzo[d]imidazole-4-carboxamide

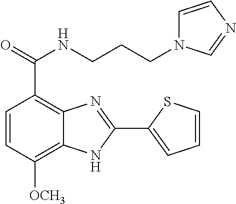

Following General Procedure B,

7-Methoxy-2-(thiophen-2-yl)-1H-benzo[d]imidazole-4-carboxylic Acid (150 mg, 0.55 mmol) was reacted with 3-(1H-imidazol-1-yl)propan-1-amine (0.14 g, 1.1 mmol) to afford the desired product (117 mg) as a light yellow oil: ESI MS m/z 382 [C₁₉H₁₉N₅O₂S+H]⁺.

### Example 76

### N-[3-(1H-Imidazol-1-yl)propyl]-7-hydroxy-2-(thiophen-2-yl)-1H-benzo[d]imidazole-4-carboxamide

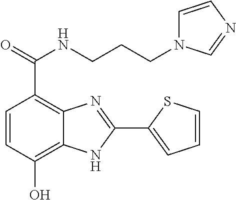

Following General Procedure C,

N-(3-(1H-imidazol-1-yl)propyl)-7-methoxy-2-(thiophen-2-yl)-1H-benzo[d]imidazole-4-carboxamide (115 mg) was reacted with boron tribromide to afford the desired product (41 mg, 20% yield) as a light yellow-brown solid: ¹H NMR (300 MHz, CD₃OD) delta 7.89-7.88 (m, 1H), 7.80-7.77 (m, 1H), 7.64-7.63 (m, 1H), 7.24-7.20 (m, 1H), 6.99 (s, 1H), 6.71 (d, 1H, J=8.3 Hz), 4.29-4.25 (m, 2H), 3.53-3.49 (m, 2H), 2.24-2.19 (m, 2H); ESI MS m/z 368 [C₁₈H₁₇N₅O₂S+H]⁺; HPLC 97.3% (AUC), t_{R}=9.69 min.

### Example 77

### 2-(Furan-2-yl)-7-hydroxy-N-phenethyl-1H-benzo[d]imidazole-4-carboxamide

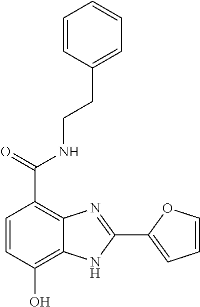

To a solution of 2-(furan-2-yl)-7-hydroxy-1H-benzo[d]imidazole-4-carboxylic acid (150 mg, 0.58 mmol) in DMF (5 mL) was added HATU (0.26 g, 0.69 mmol) 2-phenylethanamine (0.14 g, 1.2 mmol), and DIPEA (0.22 g, 1.7 mmol) and the reaction mixture was stirred at 80 degrees for 16 h. The reaction mixture was cooled to room temperature, diluted with satd. aq NaHCO₃ (50 mL), and extracted with ethyl acetate (3×30 mL). The combined organic layers were washed with satd. aq NaCl (50 mL), concentrated, and purified by flash chromatography (silica, 0-15% methanol/dichloromethane) to afford the desired product (15 mg, 6.7% yield) as a light yellow solid: ¹H NMR (500 MHz, CD₃OD) delta 7.80 (d, J=4.3 Hz, 1H), 7.75 (s, 1H), 7.35 (d, J=3.7 Hz, 1H), 7.27 (m, J=7.5 Hz, 2H), 7.19 (t, J=7.6 Hz, 1H), 7.08 (d, J=3.0 Hz, 1H), 6.70-6.66 (m, 2H), 3.79 (t, J=7.0 Hz, 2H), 3.00 (t, J=7.0 Hz, 2H); ESI MS m/z 348 [C₂₀H₁₇N₃O₃+H]⁺; HPLC 98.6% (AUC), t_{R}=13.12 min.

### Example 78

### Synthesis of 2-(Furan-2-yl)-7-hydroxy-N-phenyl-1H-benzo[d]imidazole-4-carboxamide

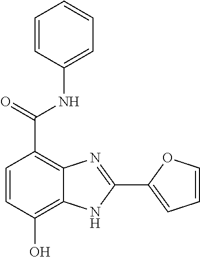

To a solution of 2-(furan-2-yl)-7-hydroxy-1H-benzo[d]imidazole-4-carboxylic acid (150 mg, 0.58 mmol) in DMF (5 mL) was added HATU (0.26 g, 0.69 mmol) aniline (0.11 g, 1.2 mmol), and DIPEA (0.22 g, 1.7 mmol) and the reaction mixture was stirred at 80 degrees for 16 h. The reaction mixture was cooled to room temperature, diluted with satd. aq NaHCO₃ (50 mL), and extracted with ethyl acetate (3×30 mL). The combined organic layers were washed with satd. aq NaCl (50 mL), concentrated, and purified by flash chromatography (silica, 0-15% methanol/dichloromethane) to afford the desired product (21 mg, 10% yield) as a light yellow solid: ¹H NMR (500 MHz, CD₃OD) delta 7.90 (d, J=8.6 Hz, 1H), 7.82 (s, 1H), 7.81 (d, J=5.8 Hz, 2H), 7.40 (t, J=14.9 Hz, 2H), 7.33 (d, J=3.3 Hz, 1H), 7.13 (t, J=14.6 Hz, 1H), 6.76 (d, J=8.2 Hz, 1H), 6.71 (s, 1H); ESI MS m/z 320 [C₁₈H₁₃N₃O₃+H]⁺; HPLC 92.7% (AUC), t_{R}=13.27 min.

### Example 79

### 7-Hydroxy-N-[3-(5-oxo-4,5-dihydro-1H-pyrazol-4-yl)propyl]-2-(thiophen-2-yl)-1H-benzo[d]imidazole-4-carboxamide

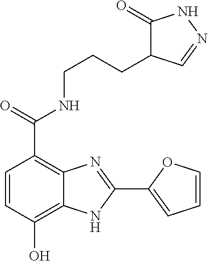

To a solution of 2-(furan-2-yl)-7-hydroxy-1H-benzo[d]imidazole-4-carboxylic acid (150 mg, 0.58 mmol) in DMF (5 mL) was added HATU (0.26 g, 0.69 mmol) 4-(3-aminopropyl)-1H-pyrazol-5(4H)-one (0.17 g, 1.2 mmol) and DIPEA (0.22 g, 1.7 mmol) and the reaction mixture was stirred at 80 degrees for 16 h. The reaction mixture was cooled to room temperature, diluted with satd. aq NaHCO₃ (50 mL), and extracted with ethyl acetate (3×30 mL). The combined organic layers were washed with satd. aq NaCl (50 mL), concentrated, and purified by flash chromatography (silica, 0-15% methanol/dichloromethane) to afford the desired product (15 mg, 5% yield) as a light yellow solid: ¹H NMR (300 MHz, CD₃OD) delta 7.76-7.59 (m, 2H), 7.35 (s, 1H), 7.27 (d, J=3.4 Hz, 1H), 6.71-6.66 (m, 2H) 3.54 (t, J=15.7 Hz, 2H), 2.57 (t, J=14.5 Hz, 2H), 1.96-1.92 (m, 2H); ESI MS m/z 368 [C₁₈H₁₇N₅O₄+H]⁺; HPLC>95.9% (AUC), t_{R}=9.59 min.

### Example 80

### N-(3,4-Dihydroxyphenethyl)-2-(furan-2-yl)-7-hydroxy-1H-benzo[d]imidazole-4-carboxamide

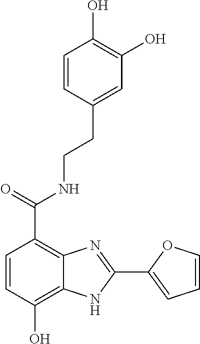

To a solution of 2-(furan-2-yl)-7-hydroxy-1H-benzo[d]imidazole-4-carboxylic acid (150 mg, 0.58 mmol) in DMF (5 mL) was added HATU (0.26 g, 0.69 mmol), 4-(2-aminoethyl)benzene-1,2-diol (0.18 g, 1.2 mmol), and DIPEA (0.22 g, 1.7 mmol) and the reaction mixture was stirred at 80 degrees for 16 h. The reaction mixture was cooled to room temperature, diluted with satd. aq NaHCO₃ (50 mL), and extracted with ethyl acetate (3×30 mL). The combined organic layers were washed with satd. aq NaCl (50 mL), concentrated, and purified by flash chromatography (silica, 0-15% methanol/dichloromethane) to afford the desired product (20 mg, 6% yield) as an off-white solid: ¹H NMR (500 MHz, CD₃OD) delta 7.80 (d, J=8.1 Hz, 1H), 7.74 (s, 1H), 7.08 (s, 1H), 6.76 (s, 1H), 6.68-6.66 (m, 4H), 3.74 (t, J=6.5 Hz, 2H), 2.84 (t, J=6.6 Hz, 2H); ESI MS m/z 380 [C₂₀H₁₇N₃O₅+H]⁺.

### Example 81

### 2-(Furan-2-yl)-7-methoxy-N-(2-(1-methyl-1H-pyrrol-2-yl)ethyl)-1H-benzo[d]imidazole-4-carboxamide

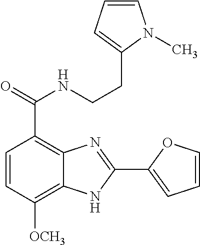

Following General Procedure B, 2-(furan-2-yl)-7-methoxy-1H-benzo[d]imidazole-4-carboxylic acid (0.15 g, 0.57 mmol) was reacted with 2-(1-methyl-1H-pyrrol-2-yl)ethanamine (0.14 g, 1.2 mmol) to afford the desired product (135 mg) as a light yellow oil: ESI MS m/z 365 [C₂₀H₂₀N₄O₃+H]⁺.

### Example 82

### N-[2-(3,5-Dimethylisoxazol-4-yl)ethyl]-2-(furan-2-yl)-7-methoxy-1H-benzo[d]imidazole-4-carboxamide

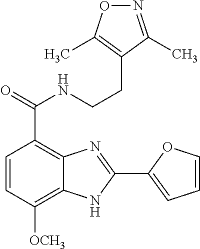

Following General Procedure B, 2-(furan-2-yl)-7-methoxy-1H-benzo[d]imidazole-4-carboxylic acid (0.15 g, 0.57 mmol) was reacted with 2-(3,5-dimethylisoxazol-4-yl)ethanamine (0.17 g, 1.2 mmol) to afford the desired product (230 mg) as a light yellow oil: ESI MS m/z 381 [C₂₀H₂₀N₄O₄+H]⁺.

### Example 83

### 2-(Furan-2-yl)-7-methoxy-N-(thiazol-2-yl)-1H-benzo[d]imidazole-4-carboxamide

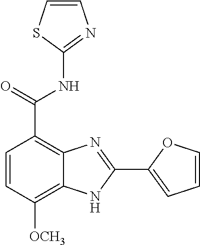

Following General Procedure B, 2-(furan-2-yl)-7-methoxy-1H-benzo[d]imidazole-4-carboxylic acid (17 mg, 0.64 mmol) was reacted with thiazol-2-amine (0.12 g, 1.2 mmol) to afford the desired product (194 mg) as a brown solid: ESI MS m/z 341 [C₁₆H₁₂N₄O₃S+H]⁺.

### Example 84

### 2-(Furan-2-yl)-7-hydroxy-N-[2-(1-methyl-1H-pyrrol-2-yl)ethyl]-1H-benzo[d]imidazole-4-carboxamide

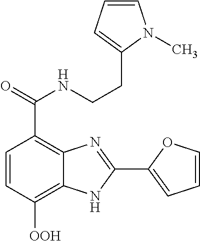

Following General Procedure C,

2-(Furan-2-yl)-7-methoxy-N-(2-(1-methyl-1H-pyrrol-2-yl)ethyl)-1H-benzo[d]imidazole-4-carboxamide (135 mg) was reacted with boron tribromide to afford the desired product (18 mg, 7% yield) as a light yellow-brown solid: ¹H NMR (300 MHz, CD₃OD) delta 7.81 (d, J=8.3 Hz, 1H), 7.75 (s, 1H), 7.19 (d, J=3.3 Hz, 1H), 6.71-6.66 (m, 2H), 6.56 (t, J=4.3 Hz, 1H), 6.02-5.97 (m, 2H), 3.77 (t, J=6.9 Hz, 2H), 3.60 (s, 3H), 2.96, (t, J=6.8 Hz, 2H) (ESI MS m/z 351 [C₁₉H₁₈N₄O₃+H]⁺; HPLC 96.7% (AUC), t_{R}=12.53 min.

### Example 85

### N-(2-(3,5-dimethylisoxazol-4-yl)ethyl)-2-(furan-2-yl)-7-hydroxy-1H-benzo[d]imidazole-4-carboxamide

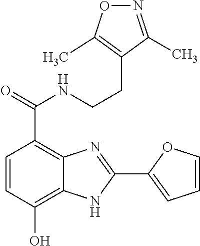

Following General Procedure C,

N-[2-(3,5-Dimethylisoxazol-4-yl)ethyl]-2-(furan-2-yl)-7-methoxy-1H-benzo[d]imidazole-4-carboxamide (230 mg) was reacted with boron tribromide to afford the desired product (18 mg, 7% yield) as an off-white solid: ¹H NMR (500 MHz, CD₃OD) delta 7.77-7.75 (m, 2H), 7.31 (bs, 1H), 6.75-6.69 (m, 2H) 3.62 (t, J=12.6 Hz, 2H), 2.74 (t, J=11.8 Hz, 2H), 2.27 (s, 3H), 2.24 (s, 3H) ESI MS m/z 367 [C₁₉H₁₈N₄O₄+H]⁺; HPLC 96.8% (AUC), t_{R}=11.65 min.

### Example 86

### 2-(Furan-2-yl)-7-hydroxy-N-(thiazol-2-yl)-1H-benzo[d]imidazole-4-carboxamide

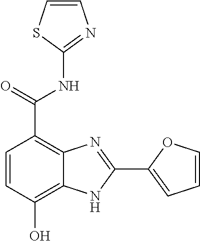

Following General Procedure C,

2-(Furan-2-yl)-7-methoxy-N-(thiazol-2-yl)-1H-benzo[d]imidazole-4-carboxamide (194 mg) was reacted with boron tribromide to afford the desired product (25 mg, 12% yield) as a light yellow solid: ¹H NMR (300 MHz, CD₃OD) delta 7.95 (d, J=8.4 Hz, 1H), 7.63 (d, J=3.6 Hz, 1H), 7.53 (d, J=3.6 Hz, 1H), 7.19 (d, J=3.6 Hz, 1H), 6.80 (d, J=8.4 Hz, 1H), 6.73-6.71 (m, 1H); ESI MS m/z 327 [C₁₅H₁₀N₄O₃S+H]⁺; HPLC>99% (AUC), t_{R}=12.88 min.

### Example 87

### 2-(Furan-2-yl)-7-hydroxy-N-[2-(5-nitropyridin-2-ylamino)ethyl]-1H-benzo[d]imidazole-4-carboxamide

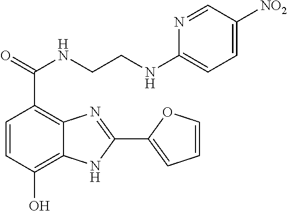

To a suspension of 2-(furan-2-yl)-7-hydroxy-1H-benzo[d]imidazole-4-carboxylic acid (0.50 g, 2.0 mmol) in DMF (4 mL) was added N¹-(5-nitropyridin-2-yl)ethane-1,2-diamine (0.41 g, 2.2 mmol), HATU (0.93 g, 2.5 mmol), DMAP (0.025 g, 0.20 mmol), and diisopropylethylamine (0.79 g, 6.2 mmol) and the reaction mixture stirred for 16 h at 50 degrees. The reaction mixture was quenched by the addition of water (25 mL) and extracted with dichloromethane (3×50 mL). The combined organic layers were dried over NaSO₄, filtered, and concentrated under reduced pressure. The crude residue was triturated in dichloromethane (10 mL) to afford the first batch of 50. The filtrate was concentrated and purified by flash chromatography (silica, 0-20% methanol/dichloromethane) and combined with the first batch to afford the desired product (0.30 g, 36% yield) as a yellow solid: ESI MS m/z 409 [C₁₉H₁₆N₆O₅+H]⁺.

### Example 88

### N-[2-(5-Aminopyridin-2-ylamino)ethyl]-2-(furan-2-yl)-7-hydroxy-1H-benzo[d]imidazole-4-carboxamide hydrochloride

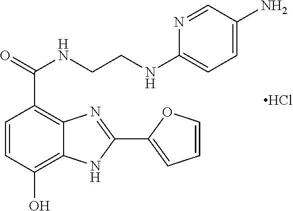

To a solution of 2-(Furan-2-yl)-7-hydroxy-N-[2-(5-nitropyridin-2-ylamino)ethyl]-1H-benzo[d]imidazole-4-carboxamide (0.30 g, 0.73 mmol) in ethanol (7 mL) and 6 N HCl (7 mL) was added iron powder (0.20 g, 3.7 mmol) and the reaction mixture was heated at reflux for 4 h. The reaction mixture was concentrated under reduced pressure to afford the desired product which was used immediately in the next step without further purification: ESI MS m/z 379 [C₁₉H₁₈N₆O₃+H]⁺.

### Example 89

### 2-(Furan-2-yl)-7-hydroxy-N-{2-[5-(4-methylphenylsulfonamido)pyridin-2-ylamino]ethyl}-1H-benzo[d]imidazole-4-carboxamide

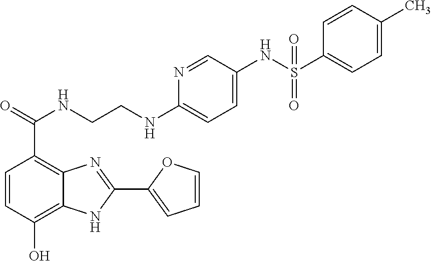

To a solution of N-[2-(5-Aminopyridin-2-ylamino)ethyl]-2-(furan-2-yl)-7-hydroxy-1H-benzo[d]imidazole-4-carboxamide hydrochloride (0.73 mmol) in DMF (7 mL) was added DIPEA (0.47 g, 3.7 mmol) and p-toluenesulfonyl chloride (0.15 g, 0.80 mmol) and the reaction mixture was stirred for 16 h at room temperature. The reaction mixture was quenched by the addition of water (25 mL) and the black solid was removed by vacuum filtration. The filtrate was concentrated under reduced pressure and the crude residue was triturated in methanol and filtered. The filtrate was concentrated and purified by flash chromatography (silica, 0-20% methanol/dichloromethane) to afford crude product. The crude product was purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to afford the desired product (42 mg, 11% yield) as a white solid: ¹H NMR (500 MHz, CD₃OD) delta 7.74 (bs, 1H), 7.51-7.49 (m, 3H), 7.25-7.23 (m, 2H), 7.15 (dd, J=9.0, 2.5 Hz, 2H), 6.69 (d, J=8.5 Hz, 1H), 6.66 (d, J=1.5 Hz, 2H), 6.49 (d, J=9.0 Hz, 1H), 3.71 (bs, 2H), 3.54 (bs, 2H), 2.35 (s, 3H); ESI MS m/z 533 [C₂₆H₂₄N₆O₅S+H]⁺; HPLC>99% (AUC), t_{R}=11.73 min.

### Example 90

### N-[2-(1H-Imidazol-5-yl)ethyl]-7-fluoro-2-(thiophen-2-yl)-1H-benzo[d]imidazole-4-carboxamide

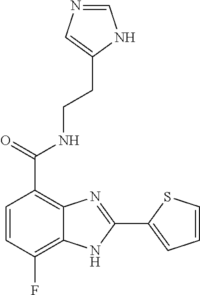

To a solution of 7-fluoro-2-(thiophen-2-yl)-1H-benzo[d]imidazole-4-carboxylic acid (100 mg, 0.38 mmol) in DMF (3 mL) was added HATU (160 mg, 0.41 mmol), DIPEA (0.35 mL, 1.9 mmol), and 2-(1H-imidazol-4-yl)ethanamine (100 mg, 0.57 mmol) and the reaction mixture stirred at 60 degrees for 5 h. The reaction mixture was cooled to room temperature, concentrated, and the crude residue was purified by column chromatography (silica, 5:95 methanol/methylene chloride) to afford the desired product (110 mg, 43%) as an off-white solid: ¹H NMR (500 MHz, DMSO) delta 9.42 (bs, 1H), 8.10 (s, 1H), 7.82 (d, J=4.6 Hz, 1H), 7.78 (s, 1H), 7.57 (s, 1H), 7.26 (t, J=8.40 Hz, 1H), 7.15 (t, J=9.3 Hz, 1H), 6.91 (s, 1H), 3.67-3.65 (m, 2H), 2.84 (t, J=6.9 Hz, 2H); ESI MS m/z 356 [C₁₇H₁₄FN₅OS+H]⁺; HPLC 98.8% (AUC), t_{R}=9.41 min.

### Example 91

### 2-Cyclopropyl-N-(4-hydroxyphenyl)-4-methoxy-1H-benzo[d]imidazole-7-carboxamide

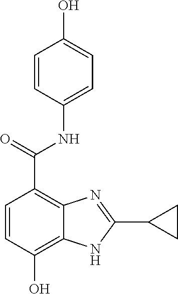

Following General procedure A, 2-cyclopropyl-7-hydroxy-1H-benzo[d]imidazole-4-carboxylic acid (40 mg, 0.18 mmol) was reacted with 4-aminophenol (31 mg, 0.28 mmol) to afford the desired product 1 (18 mg, 32% yield) as a brown yellow solid: ¹H NMR (500 MHz, CD₃OD) delta 7.76 (d, J=8.5 Hz, 1H), 7.51 (d, J=8.5 Hz, 2H), 6.80 (m, 2H), 6.65 (d, J=8.5 Hz, 1H), 2.21 (m, 1H), 1.23-1.18 (m, 4H); ESI MS m/z 310 [C₁₇H₁₅N₃O₃+H]⁺; HPLC>99% (AUC), t_{R}=9.06 min.

### Example 92

### 4-Hydroxy-N-(4-hydroxyphenethyl)-2-phenyl-1H-benzo[d]imidazole-7-carboxamide

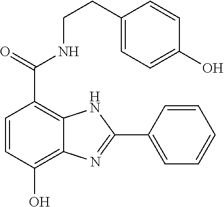

Following General procedure A, 7-hydroxy-2-phenyl-1H-benzo[d]imidazole-4-carboxylic acid (63 mg, 0.25 mmol) was reacted 4-aminophenol (52 mg, 0.38 mmol) to afford the desired product (20 mg, 21% yield) as a light brown solid: ¹H NMR (500 MHz, DMSO-d₆) delta 13.30 (s, 1H), 10.71 (s, 1H), 9.69-9.67 (m, 1H), 9.20 (s, 1H), 8.13-8.12 (m, 2H), 7.73 (d, 8.5 Hz, 1H), 7.58-7.55 (m, 3H), 7.15 (d, J=8.5 Hz, 2H), 6.74-6.71 (m, 3H), 3.72-3.69 (m, 2H), 3.32 (bs, 1H), 2.51-2.50 (m, 2H); ESI MS m/z 374 [C₂₂H₁₉N₃O₃+H]⁺; HPLC>99% (AUC), t_{R}=11.91 min.

### Example 93

### N-(4-Aminophenethyl)-4-hydroxy-2-phenyl-1H-benzo[d]imidazole-7-carboxamide

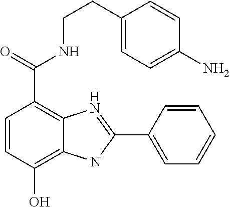

Following General procedure A, 7-hydroxy-2-phenyl-1H-benzo[d]imidazole-4-carboxylic acid (63 mg, 0.25 mmol) was reacted with benzene-1,4-diamine (52 mg, 0.38 mmol) to afford the desired product (15 mg, 16% yield) as a light brown solid: ¹H NMR (500 MHz, DMSO-d₆) delta 13.28 1 (s, 1H), 10.70 (s, 1H), 9.68 (s, 1H), 8.16 (d, J=7.5 Hz, 2H), 7.72 (d, J=6.0 Hz, 1H), 7.60-7.57 (m, 2H), 7.52 (d, J=7.5 Hz, 1H), 7.02 (d, J=6.0 Hz, 2H), 6.72 (d, J=8.5 Hz, 1H), 6.54 (d, J=8.5 Hz, 2H), 3.66 (d, J=6.0 Hz, 2H), 2.78-2.75 (m, 2H); ESI MS m/z 373 [C₂₂H₂₀N₄O₂+H]⁺; HPLC 95.7% (AUC), t_{R}=9.07 min.

### Example 94

### 4-Hydroxy-N-phenethyl-2-phenyl-1H-benzo[d]imidazole-7-carboxamide

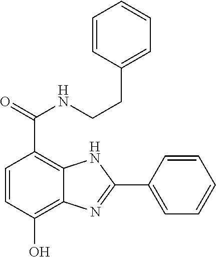

Following General procedure A, 7-hydroxy-2-phenyl-1H-benzo[d]imidazole-4-carboxylic acid (63 mg, 0.25 mmol) was reacted with 4-(2-aminoethyl)aniline (46 mg, 0.38 mmol) to afford the desired product (28 mg, 27% yield) as a white solid: ¹H NMR (500 MHz, DMSO-d₆) delta 13.30 (s, 1H), 10.71 (s, 1H), 9.70 (s, 1H), 8.12 (d, J=5.5 Hz, 2H), 7.72 (d, J=8.0 Hz, 1H), 7.56-7.52 (m, 3H), 7.37-7.22 (m, 5H), 6.72 (d, J=8.0 Hz, 1H), 3.76 (d, J=5.5 Hz, 2H), 2.96-2.93 (m, 2H); ESI MS m/z 358 [C₂₂H₁₉N₃O₂+H]⁺; HPLC>99% (AUC), t_{R}=14.39 min.

### Example 95

### 2-Cyclopentyl-4-hydroxy-N-(4-hydroxyphenethyl)-1H-benzo[d]imidazole-7-carboxamide

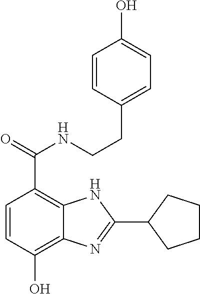

Following General procedure A, 2-cyclopentyl-7-hydroxy-1H-benzo[d]imidazole-4-carboxylic acid (80 mg, 0.28 mmol) was reacted with 4-(2-aminoethyl)phenol (58 mg, 0.42 mmol) to afford the desired product (28 mg, 27% yield) as a white solid: ¹H NMR (500 MHz, DMSO-d₆) delta 12.56c (s, 1H), 10.46 (s, 1H), 9.71 (s, 1H), 9.13 (s, 1H), 7.61 (d, J=8.5 Hz, 1H), 7.08 (d, J=8.5 Hz, 2H), 6.67-6.62 (m, 3H), 3.59-3.31 (m, 2H), 3.25-3.23 (m, 1H), 2.51-2.49 (m, 2H), 2.05-2.01 (m, 2H), 1.85-1.66 (m, 6H); ESI MS m/z 366 [C₂₁H₂₃N₃O₃+H]⁺; HPLC>99% (AUC), t_{R}=10.44 min.

### Example 96

### N-(4-Aminophenethyl)-2-cyclopentyl-4-hydroxy-1H-benzo[d]imidazole-7-carboxamide

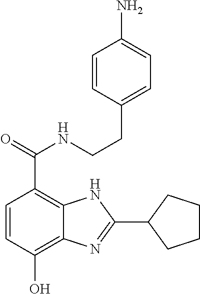

Following General procedure A, 2-cyclopentyl-7-hydroxy-1H-benzo[d]imidazole-4-carboxylic acid (80 mg, 0.28 mmol) was reacted with 4-(2-aminoethyl)aniline (58 mg, 0.42 mmol) to afford the desired product (25 mg, 25% yield) as an off-white solid: ¹H NMR (500 MHz, DMSO-d₆) delta 12.56 (s, 1H), 10.45 (s, 1H), 9.72-9.70 (m, 1H), 7.61 (d, J=8.5 Hz, 1H), 6.94 (d, J=8.5 Hz, 2H), 6.62 (d, J=8.5 Hz, 1H), 6.49-6.47 (m, 2H), 4.83 (bs, 2H), 3.56-3.52 (m, 2H), 3.33-3.25 (m, 1H), 2.51-2.49 (m, 2H), 2.07-2.02 (m, 2H), 1.89-1.79 (m, 4H), 1.68-1.66 (m, 2H); ESI MS m/z 365 [C₂₁H₂₄N₄O₂+H]⁺; HPLC>99% (AUC), t_{R}=7.97 min.

### Example 97

### 2-Cyclopropyl-N-(2,3-dihydroxypropyl)-4-hydroxy-1H-benzo[d]imidazole-7-carboxamide

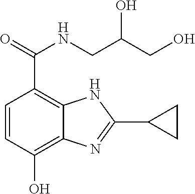

Following General procedure A, 2-cyclopropyl-7-hydroxy-1H-benzo[d]imidazole-4-carboxylic acid (55 mg, 0.25 mmol) was reacted with 3-aminopropane-1,2-diol (33 mg, 0.38 mmol) to afford the desired product (23 mg, 32% yield) as a light brown-yellow solid: ¹H NMR (500 MHz, CD₃OD) delta 7.69 (bs, 1H), 6.62-6.60 (m, 1H), 3.85-3.82 (m, 1H), 3.68 (bs, 1H), 3.60-3.59 (m, 2H), 3.51-3.47 (m, 1H), 2.15 (bs, 1H), 1.21-1.10 (m, 4H); ESI MS m/z 292 [C₁₄H₁₇N₃O₄+H]⁺; HPLC>99% (AUC), t_{R}=7.55 min.

### Example 98

### 2-Cyclopropyl-N-(2-(dimethylamino)ethyl)-4-hydroxy-1H-benzo[d]imidazole-7-carboxamide

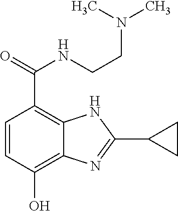

Following General procedure A, 2-cyclopropyl-7-hydroxy-1H-benzo[d]imidazole-4-carboxylic acid (55 mg, 0.25 mmol) was reacted with N¹,N¹-dimethylethane-1,2-diamine (33 mg, 0.38 mmol) to afford the desired product (35 mg, 49% yield) as a light brown-yellow solid: ¹H NMR (500 MHz, CD₃OD) delta 7.66 (d, J=8.5 Hz, 1H), 6.60 (d, J=8.5 Hz, 1H), 3.65 (t, J=6.5 Hz, 2H), 2.77 (t, J=6.5 Hz, 2H), 2.46 (s, 6H), 2.17 (bs, 1H), 1.19-1.12 (m, 4H); ESI MS m/z 289 [C₁₅H₂₀N₄O₂+H]⁺; HPLC>99% (AUC), t_{R}=6.47 min.

### Example 99

### 2-Cyclopropyl-4-methoxy-N-(4-sulfamoylphenethyl)-1H-benzo[d]imidazole-7-carboxamide

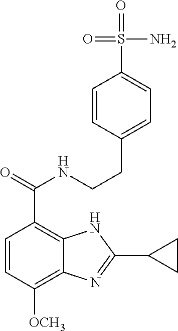

Following General procedure B, 2-cyclopropyl-7-methoxy-1H-benzo[d]imidazole-4-carboxylic acid (80 mg, 0.34 mmol) was reacted with 4-(2-aminoethyl)benzenesulfonamide (103 mg, 0.52 mmol) to afford the desired product (66 mg, 46% yield) as a brown solid: ESI MS m/z 415 [C₂₀H₂₂N₄O₄S+H]⁺.

### Example 100

### 2-Cyclopropyl-N-(4-fluorophenethyl)-4-methoxy-1H-benzo[d]imidazole-7-carboxamide

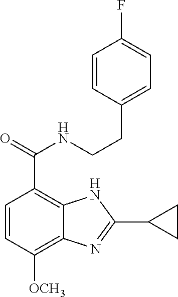

Following General procedure B, 2-cyclopropyl-7-methoxy-1H-benzo[d]imidazole-4-carboxylic acid (80 mg, 0.34 mmol) was reacted with 2-(4-fluorophenyl)ethanamine (72 mg, 0.52 mmol) to afford the desired product (80 mg, 66% yield) as a white solid: ESI MS m/z 354 [C₂₀H₂₀FN₃O₂+H]⁺.

### Example 101

### 2-Cyclopropyl-4-hydroxy-N-(4-sulfamoylphenethyl)-1H-benzo[d]imidazole-7-carboxamide

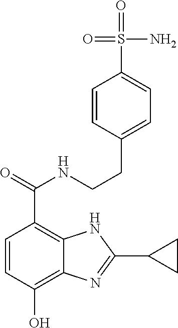

Following General procedure C,

2-Cyclopropyl-4-methoxy-N-(4-sulfamoylphenethyl)-1H-benzo[d]imidazole-7-carboxamide (60 mg, 0.15 mmol) was reacted with boron tribromide to afford the desired product (15 mg, 26% yield) as a off-white solid: ¹H NMR (500 MHz, CD₃OD) delta 7.84-7.82 (m, 2H), 7.69 (d, J=8.0 Hz, 1H), 7.48 (d, J=8.0 Hz, 2H), 6.59 (d, J=8.0 Hz, 1H), 3.81-3.79 (m, 2H), 3.06-3.03 (m, 2H), 2.08 (bs, 1H), 1.12-1.00 (m, 4H); ESI MS m/z 401 [C₁₉H₂₀N₄O₄S+H]⁺; HPLC 96.5% (AUC), t_{R}=9.05 min.

### Example 102

### 2-Cyclopropyl-N-(4-fluorophenethyl)-4-hydroxy-1H-benzo[d]imidazole-7-carboxamide

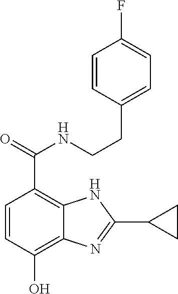

Following General procedure C,

2-Cyclopropyl-N-(4-fluorophenethyl)-4-methoxy-1H-benzo[d]imidazole-7-carboxamide (60 mg, 0.17 mmol) was reacted with boron tribromide to afford the desired product (15 mg, 26% yield) as a off-white solid: ¹H NMR (500 MHz, CD₃OD) delta 7.69 (bs, 1H), 7.30-7.27 (m, 2H), 7.02-6.99 (m, 2H), 6.60 (d, J=8.0 Hz, 1H), 3.72 (bs, 2H), 2.94-2.91 (m, 2H), 2.11 (bs, 1H), 1.00-1.00 (m, 4H); ESI MS m/z 340 [C₁₉H₁₈FN₃O₂+H]⁺; HPLC 98.9% (AUC), t_{R}=11.49 min.

### Example 103

### tert-Butyl 2-cyclopropyl-7-methoxy-1H-benzo[d]imidazol-4-ylcarbamate and 1,3-bis(2-cyclopropyl-7-methoxy-1H-benzo[d]imidazol-4-yl)urea

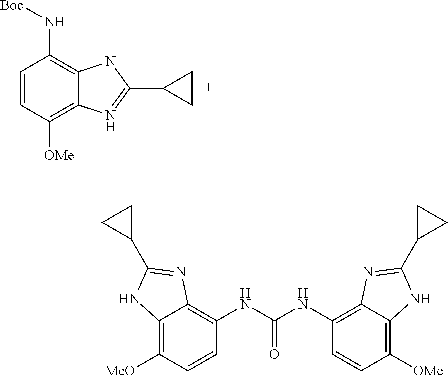

To the solution of 2-cyclopropyl-7-methoxy-1H-benzo[d]imidazole-4-carboxylic acid (1.4 g, 6.0 mmol) in 1,4-dioxane (100 mL) was added t-butanol (4 mL), triethylamine (2.0 mL, 15 mmol), and DPPA (2.5 g, 9.0 mmol) and the reaction mixture was stirred at room temperature for 2 h. Additional t-butanol (4 mL) was added and the reaction mixture was heated at 100 degrees for 18 h. The reaction mixture was cooled to room temperature, concentrated, diluted with ice water (40 mL), and the mixture was extracted with EtOAc (3×60 mL). The combined organic layers were washed with 5% aq NaHCO₃ (50 mL), brine (50 mL), dried over Na₂SO₄, and concentrated to afford a mixture of products (1.4 g) as dark blue solid which was carried forward without further purification: ESI MS m/z 304 [C₁₆H₂₁N₃O₃+H]⁺ and ESI MS m/z 433 [C₂₃H₂₄N₆O₃+H]⁺.

### Example 104

### 2-Cyclopropyl-7-methoxy-1H-benzo[d]imidazol-4-amine

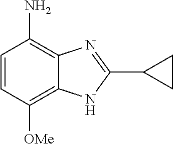

To a solution of tert-butyl 2-cyclopropyl-7-methoxy-1H-benzo[d]imidazol-4-ylcarbamate and 1,3-bis(2-cyclopropyl-7-methoxy-1H-benzo[d]imidazol-4-yl)urea (1.4 g) in 1,4-dioxane (30 mL) was added a solution of KOH (1.3 g, 24 mmol) in water (5 mL) and the reaction mixture was heated at reflux for 3 h. The reaction mixture was cooled to room temperature, concentrated, and diluted with ice water (30 mL). The pH of the mixture was adjusted to 7 using glacial acetic acid followed by extraction with EtOAc (3×80 mL). The combined organic layers were dried over Na₂SO₄ and concentrated. The crude residue was dissolved in CH₂Cl₂ (5 mL) and cooled to 0 degree followed by the addition of TFA (2 mL). The reaction mixture was stirred at room temperature for 2 h, concentrated, and diluted with ice water (20 mL). The pH of the mixture was adjusted to 7 using glacial acetic acid followed by extraction with EtOAc (3×60 mL). The combined organic layers were dried over Na₂SO₄, concentrated, and the residue was purified by flash chromatography (silica gel, 33-50% EtOAc/Hexanes) to afford the desired product (0.88 g, 72% yield) as dark blue solid: ESI MS m/z 204 [C₁₁H₁₃N₃O+H]⁺.

### Example 105

### N-(2-Cyclopropyl-7-methoxy-1H-benzo[d]imidazol-4-yl)-2-(4-methoxyphenyl)acetamide

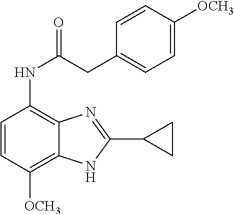

To the solution of 2-cyclopropyl-7-methoxy-1H-benzo[d]imidazol-4-amine (50 mg, 0.24 mmol) in THF (5 mL) was added triethylamine (48 micro L, 0.36 mmol) and 2-(4-methoxyphenyl)acetyl chloride (44 mg, 0.24 mmol) and the reaction mixture was stirred at room temperature for 30 min. The reaction mixture was diluted with EtOAc (20 mL) and washed with 5% aq NaHCO₃ (50 mL) and brine (50 mL). The layers were separated and the organic layer was dried over Na₂SO₄, concentrated, and the residue was purified by preparative HPLC (C18 silica, 10-90% acetonitrile/water with 0.05% TFA) to afford the desired product (60 mg, 71% yield) as dark purple-blue solid: ESI MS m/z 352 [C₂₀H₂₁N₃O₃+H]⁺.

### Example 106

### 1-(2-Cyclopropyl-7-methoxy-1H-benzo[d]imidazol-4-yl)-3-(4-methoxyphenyl)urea

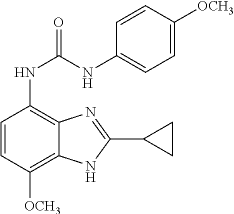

To the solution of 2-cyclopropyl-7-methoxy-1H-benzo[d]imidazol-4-amine (50 mg, 0.24 mmol) in THF (5 mL) was added triethylamine (48 micro L, 0.36 mmol) and 4-methoxyphenylcarbamic chloride (44 mg, 0.24 mmol) and the reaction mixture was stirred at room temperature for 30 min. The reaction mixture was diluted with EtOAc (20 mL) and washed with 5% aq NaHCO₃ (50 mL) and brine (50 mL). The layers were separated and the organic layer was dried over Na₂SO₄, concentrated, and the residue was purified by preparative HPLC (C18 silica, 10-90% acetonitrile/water with 0.05% TFA) to afford the desired product (66 mg, 78% yield) as dark purple-blue solid: ESI MS m/z 353 [C₁₉H₂₀N₄O₃+H]⁺.

### Example 107

### N-(2-cyclopropyl-7-hydroxy-1H-benzo[d]imidazol-4-yl)-2-(4-hydroxyphenyl)acetamide

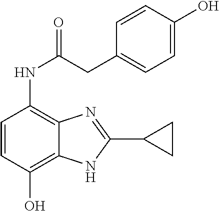

To the solution of

N-(2-cyclopropyl-7-methoxy-1H-benzo[d]imidazol-4-yl)-2-(4-methoxyphenyl)acetamide (45 mg, 0.13 mmol) in CH₂Cl₂ (15 mL) was added BBr₃ (2.1 mL, 1 M in CH₂Cl₂) and the reaction mixture was stirred at room temperature for 18 h. The reaction mixture was poured into ice water (15 mL) and the pH was adjusted to 6 using conc. NH₄OH. The reaction mixture was extracted with EtOAc (3×20 mL) and the combined organic layers were washed with 5% aq NaHCO₃ (50 mL) and brine (50 mL). The layers were separated and the organic layer was dried over Na₂SO₄, concentrated, and the residue was purified by preparative HPLC (C18 silica, 10-90% acetonitrile/water with 0.05% TFA) to afford the desired product (22 mg, 53% yield) as light purple-blue solid: ¹H NMR (500 MHz, CD₃OD) delta 7.28-7.20 (m, 3H), 6.77-6.75 (m, 2H), 6.50 (d, J=8.5 Hz, 1H), 3.64 (s, 2H), 2.15 (bs, 1H), 1.13-1.11 (m, 4H); ESI MS m/z 324 [C₁₈H₁₇N₃O₃+H]⁺; HPLC 95.7% (AUC), t_{R}=8.40 min.

### Example 108

### 1-(2-Cyclopropyl-7-hydroxy-1H-benzo[d]imidazol-4-yl)-3-(4-hydroxyphenyl)urea

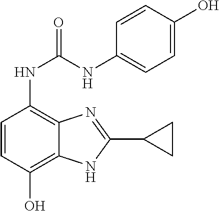

To the solution of

1-(2-cyclopropyl-7-methoxy-1H-benzo[d]imidazol-4-yl)-3-(4-methoxyphenyl)urea (50 mg, 0.13 mmol) in CH₂Cl₂ (15 mL) was added BBr₃ (2.13 mL, 1 M in CH₂Cl₂) and the reaction mixture was stirred at room temperature for 18 h. The reaction mixture was poured into ice water (15 mL) and the pH was adjusted to 6 using conc. NH₄OH. The reaction mixture was extracted with EtOAc (3×20 mL) and the combined organic layers were washed with 5% NaHCO₃ (50 mL), brine (50 mL), dried over Na₂SO₄, concentrated, and the residue was purified by preparative HPLC (C18 silica, 10-90% acetonitrile/water with 0.05% TFA) to afford the desired product (19 mg, 42% yield) as a light blue solid: ¹H NMR (500 MHz, CD₃OD) delta 7.21 (d, J=9.0 Hz, 2H), 7.05 (bs, 1H), 6.73 (d, J=9.0 Hz, 2H), 6.52 (d, J=8.0 Hz, 1H), 2.17-2.13 (m, 1H), 1.13-1.09 (m, 4H); ESI MS m/z 325 [C₁₇H₁₆N₄O₃+H]⁺; HPLC 95.6% (AUC), t_{R}=8.99 min.

### Example 109

### N-[2-(1H-Imidazol-5-yl)ethyl]-7-(benzyloxy)-1H-indole-3-carboxamide

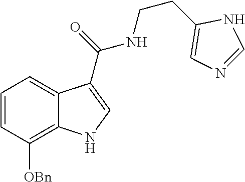

To a solution of 7-(benzyloxy)-1H-indole (1.0 g, 4.5 mmol) in DMF (10 mL) was added TFAA (2.0 g, 9.0 mmol) and the reaction mixture was stirred at room temperature for 1 h. The reaction mixture was quenched by the addition of water (50 mL) and extracted with EtOAc (3×30 mL). The combined organic layers were dried over Na₂SO₄, concentrated, and the crude residue was diluted in 6 N NaOH (15 mL) and ethanol (15 ml) and heated at reflux for 18 h. The reaction mixture was cooled to room temperature and acidified to pH 2 using 6 N HCl. The resulting solids were filtered and dried to obtain the crude acid (1.0 g) as an off-white solid. The crude acid intermediate (0.5 g) was dissolved in DMF (5 mL) followed by the addition of HATU (0.84 g, 2.2 mmol), DIPEA (1.2 mL, 6.6 mmol), histamine (0.50 g, 4.5 mmol) and the reaction mixture was stirred at room temperature for 18 h. The reaction mixture was diluted with water (20 mL) and extracted with EtOAc (3×30 mL). The combined organic layers were dried over Na₂SO₄, concentrated, and the residue was triturated with CH₂Cl₂ (20 mL). The solids were filtered to afford the desired product (0.15 g, 19% for two steps): ¹H NMR (300 MHz, CD₃OD) delta 7.79 (s, 1H), 7.59-7.51 (m, 4H), 7.47-7.28 (m, 3H), 7.04 (t, J=7.8 Hz, 1H), 6.88 (bs, 1H), 6.79 (d, J=7.8 Hz, 1H), 5.24 9s, 2H), 3.62 (t, J=7.2 Hz, 2H), 2.91 (t, J=7.2 Hz, 2H); ESI MS m/z 361 [C₂₁H₂₀N₄O₂+H]⁺.

### Example 110

### N-[2-(1H-Imidazol-5-yl)ethyl]-7-hydroxy-1H-indole-3-carboxamide

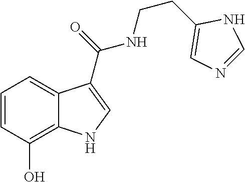

To a solution of N-(2-(1H-imidazol-5-yl)ethyl)-7-(benzyloxy)-1H-indole-3-carboxamide (0.15 g, 0.42 mmol) in methanol (20 mL) was added 10 wt % Pd on carbon (cat.) and the reaction mixture was stirred under an atmosphere (1 atm) of hydrogen at room temperature for 18 h.

The reaction mixture was filtered over diatomaceous earth and the filtrate was concentrated and purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to afford the desired product (24 mg, 22% yield): ¹H NMR (300 MHz, DMSO-d₆) delta 7.92-7.84 (m, 2H), 7.58-7.53 (m, 2H), 6.89-6.82 (m, 2H), 6.54 (d, J=7.5 Hz, 1H), 3.48-3.42 (m, 2H), 2.74 (t, J=7.2 Hz, 2H); ESI MS m/z 271 [C₁₄H₁₄N₄O₂+H]⁺.

### Example 111

### Methyl 4-Methoxy-3-(thiophene-2-carboxamido)benzoate

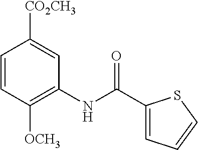

To a solution of methyl 3-amino-4-methoxybenzoate (0.31 g, 1.7 mmol) in CH₂Cl₂ (15 mL) was added EDC (0.48 g, 2.6 mmol), HOBt (0.23 g, 1.7 mmol), and thiophene-2-carboxylic acid (0.27 g, 2.1 mmol) and the reaction mixture was stirred at room temperature for 2 h. The reaction mixture was concentrated and purified by chromatography (silica gel, 0-70% EtOAc/Heptane) to afford the desired product (0.19 g, 39% yield) as an off-white solid: ESI MS m/z 292 [C₁₄H₁₃NO₄S+H]⁺.

### Example 112

### N-[5-[2-(1H-Imidazol-5-yl)ethylcarbamoyl]-2-hydroxyphenyl]thiophene-2-carboxamide

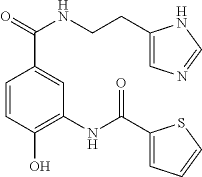

To a solution of methyl 4-methoxy-3-(thiophene-2-carboxamido)benzoate (0.19 g, 0.65 mmol) in dichloroethane (20 mL) was added boron tribromide (6.5 mL, 1.0 M in CH₂Cl₂) and the reaction mixture was heated at 80 degrees for 16 h. The reaction was incomplete by LCMS analysis, therefore, additional boron tribromide (3.3 mL, 1.0 M in CH₂Cl₂) was added and the reaction mixture was heated at 80 degrees for 24 h. The reaction mixture was cooled to room temperature, quenched by the addition of water (15 mL) and the resulting solids were filtered to afford crude hydroxy acid. The crude acid was dissolved in DMF (5 mL) followed by the addition of HATU (0.15 g, 0.46 mmol), DIPEA (0.20 mL, 1.1 mmol), and histamine (0.051 g, 0.46 mmol) and the reaction mixture was heated at 80 degrees for 18 h. The reaction mixture was cooled to room temperature, diluted with water (20 mL), and extracted with EtOAc (3×30 mL). The combined organic layers were dried over Na₂SO₄, concentrated, and the residue was purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to afford the desired product (36 mg, 27% yield for two steps): ¹H NMR (300 MHz, CD₃OD) delta 8.48 (s, 1H), 8.25 (d, J=3.0 Hz, 1H), 7.86 (d, J=3.9 Hz, 1H), 7.75-7.73 (m, 1H), 7.52 (dd, J=8.4, 2.1 Hz, 1H), 7.23-7.18 (m, 2H), 6.95 (d, J=8.4 Hz, 1H), 3.65 (t, J=6.9 Hz, 2H), 2.98 (t, J=6.9 Hz); ESI MS m/z 357 [C₁₇H₁₆N₄O₃S+H]⁺; HPLC 96.3% (AUC), t_{R}=9.15 min.

General Procedure D—synthesis of amines: To a solution of tert-butyl 2-aminoethylcarbamate (1.0 equiv) in dichloromethane (10 mL) was added triethylamine (3.0 equiv) and the requisite sulfonyl chloride or acid chloride (1.2 equiv) and the reaction mixture was stirred at room temperature for 4 h. The reaction mixture was concentrated to afford the crude intermediate were dissolved in ethyl acetate (40 mL) followed by the addition of 2 N HCl in diethyl ether (2.85 equiv). The reaction mixture was stirred for 16 h at room temperature. The resulting solids were collected by filtration to afford the desired products. These amines were used in subsequent reactions without further purification.

### Example 113

### N-(2-Aminoethyl)benzenesulfonamide Trifluoroacetic Acid Salt

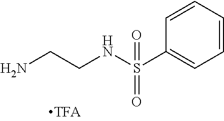

Following General Procedure D, tert-butyl 2-aminoethylcarbamate (0.50 g, 2.8 mmol) was reacted with benzenesulfonylchloride (0.54 g, 3.4 mmol) to afford the intermediate (ESI MS m/z 201 [C₁₃H₂₀N₂O₄S-Boc+H]⁺) which was treated with 2 N HCl. The reaction did not go to completion by LCMS analysis and was concentrated, dissolved in trifluoroacetic acid (5 mL) and stirred for 4 h at room temperature. The reaction mixture was concentrated under reduced pressure to afford the desired product (1.2 g, 99% yield) as a tan solid: ¹H NMR (500 MHz, DMSO-d₆) delta 7.90 (t, J=5.9 Hz, 1H), 7.83-7.81 (m, 4H), 7.69-7.63 (m, 3H), 2.94 (d, J=6.2 Hz, 2H), 2.86 (d, J=5.6 Hz, 2H).

### Example 114

### N-(2-Aminoethyl)-4-chlorobenzenesulfonamide Hydrochloride

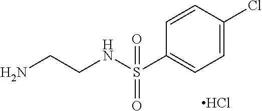

Following General Procedure D, tert-butyl 2-aminoethylcarbamate (0.50 g, 2.8 mmol) was reacted with 4-chlorobenzenesulfonylchloride (0.72 g, 3.4 mmol) to afford the intermediate (ESI MS m/z 235 [C₁₃H₁₉ClN₂O₄S-Boc+H]⁺) which was treated with 2 N HCl to afford the desired product (0.60 g, 79% yield) as a white solid: ESI MS m/z 235 [C₈H₁₁ClN₂O₂S+H]⁺.

### Example 115

### N-(2-Aminoethyl)pyridine-4-sulfonamide Dihydrochloride

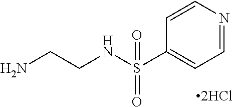

Following General Procedure D, tert-butyl 2-aminoethylcarbamate (0.54 g, 2.8 mmol) was reacted with 4-pyridylsulfonylchloride (0.73 g, 3.4 mmol) to afford the intermediate which was reacted with 2 N HCl to afford the desired product (0.72 g, 94% yield) as a white solid: ¹H NMR (500 MHz, CD₃OD) delta 9.19 (d, J=2.0 Hz, 1H), 8.97 (dd, J=5.3, 1.4 Hz, 1H), 8.66-8.64 (m, 1H), 7.99 (dd, J=8.2, 5.3 Hz, 1H), 3.24-3.19 (m, 2H), 3.11-3.09 (m, 2H).

### Examples 116

### N-(2-aminoethyl)benzamide Hydrochloride

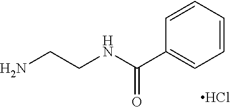

Following General Procedure D, tert-butyl 2-aminoethylcarbamate (0.50 g, 2.8 mmol) was reacted with 4-tolylbenzoylchloride (0.47 g, 3.4 mmol) to afford the intermediate (ESI MS m/z 179 [C₁₅H₂₂N₂O₃— Boc+H]⁺) which was reacted with 2 N HCl to afford the desired product (0.40 g, 67% yield) as a white solid: ¹H NMR (500 MHz, CD₃OD) delta 7.86 (t, J=8.5 Hz, 2H), 7.56-7.55 (m, 1H), 7.48 (t, J=7.5 Hz, 2H), 3.67 (t, J=5.5 Hz, 2H), 3.17 (t, J=6.0 Hz, 2H).

### Example 117

### tert-Butyl 7-methoxy-2-(thiophen-2-yl)-1H-benzo[d]imidazol-4-ylcarbamate

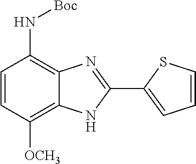

A solution 7-methoxy-2-(thiophen-2-yl)-1H-benzo[d]imidazole-4-carboxylic acid (0.60 g, 2.2 mmol), (PhO)₂P(O)N₃ (0.78 g, 3.0 mmol) and triethylamine (0.70 mL, 5.0 mmol) in 1,4-dioxane (35 mL) were stirred for 4 h at room temperature. Following the addition of t-BuOH (2 mL) the reaction mixture was stirred at 100 degrees for 16 h. The reaction mixture was cooled, concentrated, and the residue was purified by column chromatography (silica gel, methanol/methylene chloride gradient) to afford the desired product (360 mg, 47% yield) as a yellow solid: ESI MS m/z 346 [C₁₇H₁₉N₃O₃S+H]⁺.

### Example 118

### 7-methoxy-2-(thiophen-2-yl)-1H-benzo[d]imidazol-4-amine Hydrochloride

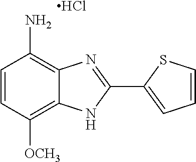

To a solution of tert-Butyl 7-methoxy-2-(thiophen-2-yl)-1H-benzo[d]imidazol-4-ylcarbamate (0.44 g, 1.2 mmol) in CH₂Cl₂ (5 mL) was added a 2.0 M HCl in diethyl ether (3.5 mL) and the reaction mixture was stirred at room temperature for 5 h. The resulting precipitate was filtered and washed with CH₂Cl₂ (2×10 mL) to afford the desired product (290 mg, 855 yield) as a white solid: ESI MS m/z 246 [C₁₂H₁₁N₃₀S+H]⁺.

### Example 119

### (E)-3-(1H-imidazol-5-yl)-N-(7-methoxy-2-(thiophen-2-yl)-1H-benzo[d]imidazol-4-yl)acrylamide

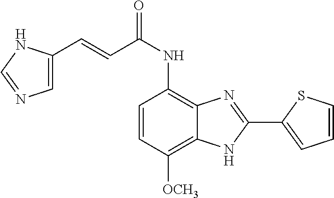

A solution of (E)-3-(1H-imidazol-5-yl)acrylic acid (0.13 g, 0.94 mmol) and HATU (0.36 g, 1.1 mmol) in THF (4 mL) was stirred at room temperature for 30 min. A solution of 7-methoxy-2-(thiophen-2-yl)-1H-benzo[d]imidazol-4-amine hydrochloride (0.18 g, 0.63 mmol) and DIPEA (0.33 mL, 1.9 mmol) in THF (4 mL) was added and the reaction mixture was heated at 60 degrees for 64 h. The reaction mixture cooled, diluted with water (50 mL), and extracted with EtOAc (2×30 mL). The combined organic layers were washed with brine (2×50 mL), dried over sodium sulfate, and concentrated. The residue was purified by column chromatography (silica gel, methanol/methylene chloride gradient) to afford the desired product (200 mg, 87% yield) as an off-white solid: ESI MS m/z 366 [C₁₈H₁₅N₅O₂S+H]⁺.

### Example 120

### (E)-3-(1H-imidazol-5-yl)-N-(7-methoxy-2-(thiophen-2-yl)-1H-benzo[d]imidazol-4-yl)acrylamide

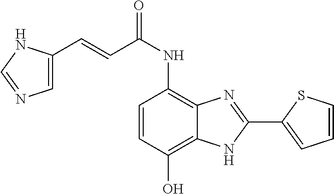

A solution of

(E)-3-(1H-imidazol-5-yl)-N-(7-methoxy-2-(thiophen-2-yl)-1H-benzo[d]imidazol-4-yl)acrylamide (0.20 g, 0.55 mmol) in CH₂Cl₂ (12 mL) was cooled to 0 degree and BBr₃ (1.6 g, 6.5 mmol) was added dropwise and the reaction mixture was warmed to room temperature and stirred for 16 h. The reaction mixture was concentrated, the residue was stirred in methanol (5 mL) and purified by preparative HPLC (C18 silica, 10-90% acetonitrile/water with 0.05% TFA). The desired fractions were concentrated and eluted through an ion-exchange column (using methanol and 7 N methanol in ammonia) to obtain the desired product (25 mg, 13% yield) as an off-white solid: ¹H NMR (500 MHz, CD₃OD) 7.81 (d, J=4.5 Hz, 1H) 7.78 (s, 1H), 7.6 (d, J=8.5 Hz, 1H), 7.59 (s, 1H), 7.51 (d, J=8.5 Hz, 1H), 7.4 (s, 1H), 7.18, (t, J=4.25 Hz, 1H), 6.78 (d, J=15.5, 1H), 6.58 (d, J=8.5 Hz, 1H); ESI MS m/z 352 [C₁₇H₁₃N₅O₂S+H]⁺.

### Example 121

### N-(7-hydroxy-2-(thiophen-2-yl)-1H-benzo[d]imidazol-4-yl)-3-(1H-imidazol-5-yl)propanamide

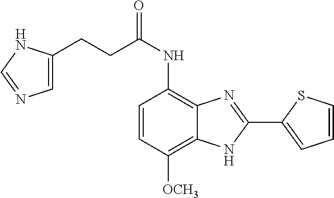

A solution of

(E)-3-(1H-imidazol-5-yl)-N-(7-methoxy-2-(thiophen-2-yl)-1H-benzo[d]imidazol-4-yl)acrylamide (19 mg, 0.054 mmol) and 10 wt % Palladium upon carbon (50 mg) in ethanol (20 mL) was placed in a parr shaker with hydrogen gas (50 psi) for 2 h. The reaction mixture was transferred into a round bottom flask, placed under an atmosphere of hydrogen gas (1 atm), and stirred for 16 h. The reaction mixture was filtered through diatomaceous earth, the filtrate was concentrated, and the residue was suspended in CH₂Cl₂ (10 mL). The resulting precipitate was filtered and dried to afford the desired product (12 mg, 63% yield) as a green solid: ¹H NMR (500 MHz, CD₃OD); 8.16 (s, 1H), 7.82, (d, J=4.0 Hz, 1H), 7.38 (d, J=5.5 Hz, 1H), 7.33 (d, J=8.0 Hz, 1H), 7.19 (t, J=4.2 Hz, 1H), 7.13 (s, 1H), 6.58 (d, J=8.5 Hz, 1H), 3.09 (t, J=7.3 Hz, 2H), 2.83 (t, J=7.5 Hz, 2H); ESI MS m/z 354 [C₁₇H₁₅N₅O₂S+H]⁺.

### Example 122

### Step 1: Synthesis of tert-butyl 3-(4-methoxy-2-nitrobenzamido)piperidine-1-carboxylate

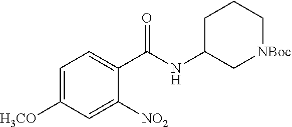

To a solution of 4-methoxy-2-nitrobenzoic acid (200 mg, 1.0 mmol) and tert-butyl 3-aminopiperidine-1-carboxylate (200 mg, 1.0 mmol) in DMF (2 mL) was added DIPEA (0.20 mL, 1.2 mmol) and HATU (460 mg, 1.2 mmol). The reaction mixture was stirred at room temperature for 18 h, diluted with water (10 mL) and ethyl acetate (30 mL), and the layers were separated. The organic phase was washed with water (20 mL), brine (20 mL), dried over Mg₂SO₄, and purified by flash chromatography (silica gel, ethyl acetate/hexanes gradient) to provide the desired product (330 mg, 88%) as a white solid: ESI MS m/z 402 [C₁₈H₂₅N₃O₆+Na]⁺.

### Step 2: Synthesis of tert-butyl 3-(2-amino-4-methoxybenzamido)piperidine-1-carboxylate

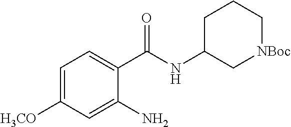

To a solution of tert-butyl 3-(4-methoxy-2-nitrobenzamido)piperidine-1-carboxylate (190 mg, 0.50 mmol) in EtOH/EtOAc (5 mL each) was added 10 wt % palladium upon carbon (20 mg) and the reaction mixture was stirred under an atmosphere of hydrogen for 3 h. The reaction mixture was filtered through diatomaceous earth and the filtrate was concentrated to afford the desired product (170 mg, quant.) as a white solid: ESI MS m/z 351 [C₁₈H₂₇N₃O₄+H]⁺.

### Step 3: Synthesis of tert-butyl 3-(4-methoxy-2-(thiophene-2-carboxamido)benzamido) piperidine-1-carboxylate

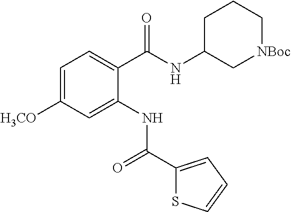

To a solution of tert-butyl 3-(2-amino-4-methoxybenzamido)piperidine-1-carboxylate (170 mg, 0.50 mmol) and DIPEA (120 mg, 1.0 mmol) in CH₂Cl₂ (5 mL) and pyridine (1 mL) at 0 degree was added thiophene-2-carbonyl chloride (88 mg, 0.60 mmol) dropwise. The reaction mixture was stirred for 18 h, concentrated, purified by flash chromatography (silica gel, ethyl acetate/hexanes gradient) to afford the desired product (200 mg, 89%) as a white solid: ESI MS m/z 460 [C₂₃H₂₉N₃O₅S+H]⁺.

### Step 4: Synthesis of N-(5-hydroxy-2-(piperidin-3-ylcarbamoyl)phenyl)thiophene-2-carboxamide

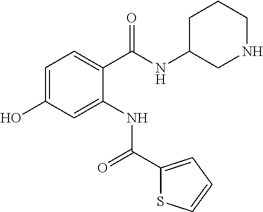

To a solution of tert-butyl 3-(4-methoxy-2-(thiophene-2-carboxamido) benzamido) piperidine-1-carboxylate (91 mg, 0.20 mmol) in CH₂Cl₂ (3 mL) at −78 degrees was added BBr₃ (2.0 mL, 1.2 mmol, 1 M in CH₂Cl₂) and the reaction mixture was warmed to room temperature and stirred for 18 h. The reaction mixture was quenched by the addition of ice and methanol (2 mL) and concentrated. The residue was purified by preparative HPLC (C18 silica, 10-90% acetonitrile/water with 0.05% TFA). The desired product was obtained as the trifluoroacetic acid salt which was eluted through an ion-exchange column (using methanol and 7 N methanol in ammonia) to afford the desired product (12 mg, 94%) as a white solid: ¹H NMR (500 MHz, DMSO-d₆) delta 12.93 (s, 1H), 10.28 (s, 1H), 8.57 (d, J=12.5 Hz, 1H), 8.09 (d, J=3.5 Hz, 1H), 7.89 (d, J=8.0 Hz, 1H), 7.82 (d, J=15.0 Hz, 1H), 7.70 (d, J=4.5 Hz, 1H), 7.28-7.25 (m, 1H), 6.58-6.55 (m, 1H), 4.19 (s, 1H), 3.57-3.55 (m, 1H), 3.46-3.33 (m, 2H), 1.91-1.87 (m, 2H), 1.68-1.30 (m, 5H); ESI MS m/z 346 [C₁₇H₁₉N₃O₃S+H]⁺; HPLC>99% (AUC), t_{R}=9.16 min.

### Examples 123

### Kinase Assay

GSK3beta activity was measured in the presence or absence of compounds using Z′-LYTE kinase assay (Rodems S M, et al., Assay Drug Dev Technol. 1: 9-19, 2002.) kit with SER/THR 9 peptide (Invitrogen) following the manufacturer's instruction. The Z′-LYTE kinase assay kit employs a fluorescence resonance energy transfer (FRET) between two fluorophores, coumarin and fluorescein, attached to each end of a substrate peptide.

Test compounds were dissolved in DMSO at 12.5 mM and then serially diluted as the DMSO concentration in the assays to be 1%. The serially diluted compounds, 0.04 ng/mcl GSKbeta (Invitrogen) and 2 mcM SER/THR 9 peptide were reacted in a reaction buffer (50 mM HEPES pH 7.5, 0.01% Brij-35, 10 mM MgCl₂, 1 mM EGTA, 15 mcM ATP). For 0% phosphorylation control, ATP was omitted from the reaction mixture. For 100% phosphorylation control, SER/THR 9 phosphopeptide was used in place of the SER/THR 9 peptide. Following 1 hour incubation at room temperature, the reaction was stopped by the addition of half assay volume of development solution and further incubated for 1 hour at room temperature. After adding the half assay volume of stop reagent, emission signals of coumarin and fluorescein were measured by Wallac EnVision 2103 multilabel reader (PerkinElmer). The extent of phosphorylation was determined according to the 0% and 100% phosphorylation control samples using the following equation:

$$\% phosphorylation = 1 - \frac{( emission ratio \times {F}_{100 \%} )}{( {C}_{0 \%} - {C}_{100 \%} ) + [ emission ratio \times ( {F}_{100 \%} - {F}_{0 \%} ) ]}$$

where:

$$emission ratio = \frac{coumarin emission signal ( 445 nm )}{fluorescein emission signal ( 520 nm )}$$

C_{100%}=coumarin emission signal of the 100% phosphorylation control
C_{0%}=coumarin emission signal of the 0% phosphorylation control
F_{100%}=fluorescein emission signal of the 100% phosphorylation control
F_{0%}=fluorescein emission signal of the 0% phosphorylation control

IC₅₀ values were calculated by nonlinear four parameter fit using SigmaPlot, version 10.0 (Systat Software, Inc.).

IC₅₀ values of the typical compounds of the present invention are shown in following table 12:

TABLE 12

| | | IC₅₀ |
|---|---|---|
| Example | | (micro |
| No. | compound | M) |
| 76 | N-[3-(1H-Imidazol-1-yl)propyl]-7-hydroxy-2-(thiophen-2-yl)-1H-benzo[d]imidazole- | 0.0017 |
| | 4-carboxamide | |
| 80 | N-(3,4-Dihydroxyphenethyl)-2-(furan-2-yl)-7-hydroxy-1H-benzo[d]imidazole-4- | 0.019 |
| | carboxamide | |
| 69 | 7-Hydroxy-N-{2-[5-(methylsulfonamido)pyridin-2-ylamino]ethyl}-2-(thiophen- | 0.022 |
| | 2-yl)-1H-benzo[d]imidazole-4-carboxamide | |
| 15 | Methyl3- | 0.022 |
| | Hydroxy-2-[7-hydroxy-2-(thiophen-2-yl)-1H-benzo[d]imidazole-4-carboxamido]propanoate | |
| 79 | 7-Hydroxy-N-[3-(5-oxo-4,5-dihydro-1H-pyrazol-4-yl)propyl]-2-(thiophen-2-yl)- | 0.03 |
| | 1H-benzo[d]imidazole-4-carboxamide | |
| 37 | N-(3,4-Dihydroxybenzyl)-7-hydroxy-2-(thiophen-2-yl)-1H-benzo[d]imidazole-4- | 0.038 |
| | carboxamide | |
| 8 | 7-Hydroxy-N-(4-sulfamoylbenzyl)-2-(thiophen-2-yl)-1H-benzo[d]imidazole-4-carboxamide | 0.052 |
| 49 | N-[2-(5-Carbamoylpyridin-2-yloxy)ethyl]-7-hydroxy-2-(thiophen-2-yl)-1H-benzo[d]imidazole- | 0.073 |
| | 4-carboxamide | |
| 42 | N-(2-Acetamidoethyl)-7-hydroxy-2-(thiophen-2-yl)-1H-benzo[d]imidazole-4-carboxamide | 0.074 |
| 70 | N-[2-(5-Acetamidopyridin-2-ylamino)ethyl]-7-hydroxy-2-(thiophen-2-yl)-1H-benzo[d]imidazole- | 0.081 |
| | 4-carboxamide | |
| 101 | 2-Cyclopropyl-4-hydroxy-N-(4-sulfamoylphenethyl)-1H-benzo[d]imidazole-7-carboxamide | 0.12 |
| 35 | 7-Hydroxy-N-[2-(1-methyl-1H-imidazol-5-yl)ethyl]-2-(thiophen-2-yl)-1H-benzo[d]imidazole- | 0.13 |
| | 4-carboxamide | |
| 11 | 7-Hydroxy-N-[2-(pyridin-2-ylamino)ethyl]-2-(thiophen-2-yl)-1H-benzo[d]imidazole- | 0.13 |
| | 4-carboxamide | |
| 97 | 2-Cyclopropyl-N-(2,3-dihydroxypropyl)-4-hydroxy-1H-benzo[d]imidazole-7-carboxamide | 0.15 |
| 17 | (R)-7-Hydroxy-N-[1-hydroxy-3-(1H-imidazol-4-yl)propan-2-yl]-2-(thiophen-2-yl)- | 0.17 |
| | 1H-benzo[d]imidazole-4-carboxamide | |
| 12 | 7-Hydroxy-N-[3-(2-hydroxyethylamino)propyl]-2-(thiophen-2-yl)-1H-benzo[d]imidazole- | 0.18 |
| | 4-carboxamide | |
| 36 | N-[2-(dimethylamino)ethyl]-7-hydroxy-2-(thiophen-2-yl)-1H-benzo[d]imidazole- | 0.18 |
| | 4-carboxamide | |
| 43 | N-[3-(Isopropylamino)propyl]-7-hydroxy-2-(thiophen-2-yl)-1H-benzo[d]imidazole- | 0.22 |
| | 4-carboxamide | |
| 62 | N-[2-(1H-Imidazol-2-yl)ethyl]-7-hydroxy-2-(thiophen-2-yl)-1H-benzo[d]imidazole- | 0.26 |
| | 4-carboxamide | |
| 21 | 7-Hydroxy-N-(4-sulfamoylphenethyl)-2-(thiophen-2-yl)-1H-benzo[d]imidazole- | 0.28 |
| | 4-carboxamide | |
| 71 | (S)-2-[7-Hydroxy-2-(thiophen-2-yl)-1H-benzo[d]imidazole-4-carboxamido]-3-(1H- | 0.34 |
| | imidazol-5-yl)propanoic Acid | |
| 40 | 7-Hydroxy-N-[2-(pyridine-4-sulfonamido)ethyl]-2-(thiophen-2-yl)-1H-benzo[d]imidazole- | 0.39 |
| | 4-carboxamide | |
| 93 | N-(4-Aminophenethyl)-4-hydroxy-2-phenyl-1H-benzo[d]imidazole-7-carboxamide | 0.4 |
| 91 | 2-Cyclopropyl-N-(4-hydroxyphenyl)-4-methoxy-1H-benzo[d]imidazole-7-carboxamide | 0.4 |
| 120 | (E)-3-(1H-imidazol-5-yl)-N-(7-methoxy-2-(thiophen-2-yl)-1H-benzo[d]imidazol- | 3 |
| | 4-yl) acrylamide | |
| 121 | N-(7-hydroxy-2-(thiophen-2-yl)-1H-benzo[d]imidazol-4-yl)-3-(1H-imidazol-5-yl)propanamide | 1.7 |
| 122 | N-(5-hydroxy-2-(piperidin-3-ylcarbamoyl)phenyl)thiophene-2-carboxamide | 8.1 |

### INDUSTRIAL APPLICABILITY

The present invention provides a novel benzoimidazole compound having GSK3beta inhibitory effect. The compounds of the present invention may be used for pharmaceutical composition for inhibiting GSK3-beta. Such pharmaceutical compositions are suitable for treating or preventing diseases involving GSK3beta.

1. A compound represented by formula (I), or a salt, hydrate, solvate, or isomer thereof: 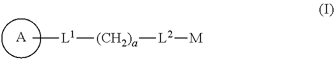
wherein
Ring A is represented by the formula: 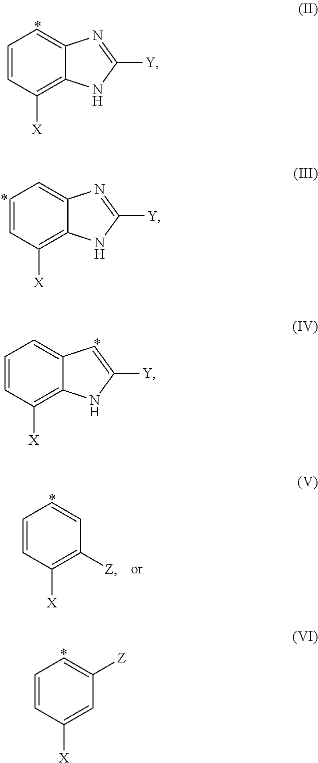
wherein
X is halogen or hydroxyl;
Y is hydrogen, phenyl, thiophen-2-yl, furan-2-yl, cyclopropyl, or cyclopentyl;
Z is a 5-10 membered heterocycle substituted carbonylamino; and
-L¹-(CH₂)ₐ-L²-M is at position *;
wherein, L¹ is —CONH—, —NHCO—, or a single bond;
L² is selected from the group consisting of —NH—, —O—, —CH(COOR¹)—, —CH(CH₂OH)—, —CH═CH— and a single bond, and wherein R¹ is hydrogen or C₁-C₆ alkyl; and
M is selected from the group consisting of hydroxyl, carboxyl, amide, C₁-C₆ alkyl, C₁-C₆ alkylcarbonyl, C₆-C₁₄ aryl, C₆-C₁₄ aryl C₁-C₆ alkyl, C₆-C₁₄ arylcarbonyl, C₆-C₁₄ arylsulfonyl, a 5-14 membered saturated, unsaturated or aromatic heterocyclic group, a 5-14 membered unsaturated or aromatic heterocyclic group substituted C₁-C₆ alkyl, a 5-14 membered unsaturated or aromatic heterocyclic group substituted sulfonyl, and —NR²R³, wherein R² and R³ are each independently C₁-C₆ alkyl;
wherein the C₁-C₆ alkyl, the C₁-C₆ alkylcarbonyl, the C₆-C₁₄ aryl, the C₆-C₁₄ aryl C₁-C₆ alkyl, the C₆-C₁₄ arylcarbonyl, the C₆-C₁₄ arylsulfonyl, the 5-14 membered unsaturated or aromatic heterocyclic group, the 5-14 membered unsaturated or aromatic heterocyclic group substituted C₁-C₆ alkyl, or the 5-14 membered unsaturated or aromatic heterocyclic group substituted sulfonyl is optionally substituted by 1-3 substituent(s), each independently selected from group A;
wherein group A is selected from the group consisting of hydroxyl, oxo, nitro, amino, amide, halogen, sulfamoyl, trifluoromethyl, p-toluenesulfonylamino, C₁-C₆ alkyl, C₁-C₆alkoxy, C₁-C₆alkylcarbonylamino, and C₁-C₆alkylsulfonylamino; and a is an integer from 0-5.

2. The compound of claim 1, which is represented by formula (I-II): 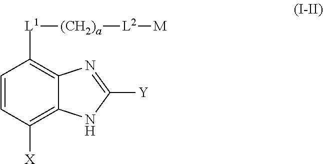
wherein
L¹ is —CONH—;
L² is a single bond; and
M is C₆-C₁₀ aryl or a 5-10 membered unsaturated or aromatic heterocycle group having 1-2 hetero atom(s) selected from the group consisting of N, O, and S, each of which is optionally substituted by 1 or 2 substituent(s) each independently selected from the group A.

3. The compound of claim 2, wherein M is phenyl, imidazole-1-yl, imdazole-2-yl, imidazole-5-yl, thiophen-2-yl, pyrole-2-yl, 1,3-thiazole-2-yl, 2-pyrazoline-4-yl, or isoxazole-4-yl, each of which is optionally substituted by 1-2 substituent(s) each independently selected from group B;
wherein group B is selected from the group consisting of fluoro, hydroxyl, oxo, amino, methyl, methoxy, and sulfamoyl.

4. The compound of claim 1, which is represented by formula (I-II): 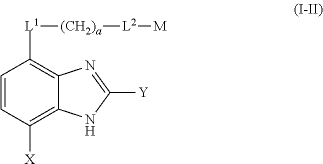
wherein
L¹ is —CONH—;
L² is —NH—; and
M is C₁-C₄ alkyl, C₁-C₄ alkylcarbonyl, C₆-C₁₀ arylcarbonyl, C₆-C₁₀ arylsulfonyl, a 5-10 membered unsaturated or aromatic heterocyclic group having 1-2 hetero atom(s) selected from the group consisting of N, O, and S or sulfonyl substituted by a 5-10 membered unsaturated or aromatic heterocyclic group having 1-2 hetero atom(s) selected from the group consisting of N, O, and S, each of which is optionally substituted by 1 or 2 substituent(s) each independently selected form the group A.

5. The compound of claim 4, wherein M is ethyl, isopropyl, methylcarbonyl, pyridine-2-yl, phenylcarbonyl, phenylsulfonyl, or 4-pyridylsulfonyl, each of which is optionally substituted by 1-2 substituent(s) each independently selected from group C;
wherein group C is selected from the group consisting of chloro, hydroxyl, methyl, methylcarbonylamino, methylsulfonylamino, and p-toluenesulfonylamino.

6. The compound of claim 1, which is represented by formula (I-II): 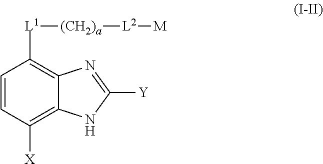
wherein
L¹ is —CONH—;
L² is —CH(COOR¹)—, wherein R¹ is hydrogen or C₁-C₄ alkyl; and
M is C₁-C₄ alkyl, C₆-C₁₀ aryl C₁-C₄ alkyl or C₁-C₄ alkyl substituted by a 5-10 membered unsaturated or aromatic heterocyclic group having 1-2 hetero atom(s) selected from the group consisting of N, O, and S, each of which is optionally substituted by 1 or 2 substituent(s) each independently selected from the group A.

7. The compound of claim 6, wherein M is methyl, phenylmethyl, indole-3-ylmethyl, or imidazole-4-ylmethyl, each of which is optionally substituted by 1-2 hydroxyl group(s).

8. The compound of claim 1, which is represented by formula (I-II): 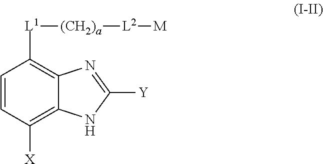
wherein
L¹ is —CONH—;
L² is —O—; and
M is C₆-C₁₀ aryl or a 5-10 membered unsaturated or aromatic heterocyclic group having 1-2 hetero atom(s) selected from the group consisting of N, O, and S, each of which is optionally substituted by 1 or 2 substituent(s) each independently selected from the group A.

9. The compound of claim 8, wherein M is phenyl or pyridine-2-yl, each of which is optionally substituted by 1 or 2 substituent(s) each independently selected from group D;
wherein group D is selected from the group consisting of amide, nitro, trifluoromethyl, and p-toluenesulfonylamino.

10. The compound of claim 1, which is represented by formula (I-II): 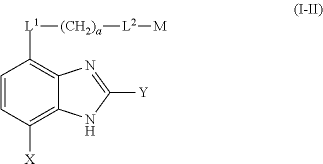
wherein
L¹ is —CONH—;
L² is —CH(CH₂OH)—; and
M is selected from the group consisting of hydroxyl, C₁-C₄ alkyl, C₆-C₁₀ aryl C₁-C₄ alkyl, and C₁-C₄ alkyl substituted by a 5-10 membered unsaturated or aromatic heterocyclic group having 1-2 hetero atom(s) selected from the group consisting of N, O, and S,
wherein the C₁-C₄ alkyl, C₆-C₁₀ aryl C₁-C₄ alkyl, and 5-10 membered unsaturated or aromatic heterocyclic group, are each optionally substituted by 1 or 2 substituent(s) each independently selected from the group A.

11. The compound of claim 10, wherein M is hydroxyl, phenylmethyl, t-butyl, or imidazole-5-ylmethyl.

12. The compound of claim 1, which is represented by formula (I-II): 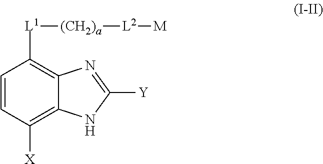
wherein
L¹ is —CONH—;
L² is a single bond; and
M is —NR²R³;
wherein R² and R³ are each independently C₁-C₄ alkyl optionally substituted by 1 or 2 substituent(s) each independently selected from the group A.

13. The compound of claim 1, which is represented by formula (I-II): 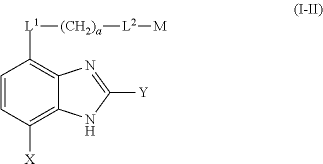
wherein
L′ is —NHCO—;
L² is —NH—, —CH═CH— or a single bond; and
M is C₆-C₁₀ aryl or a 5-10 membered unsaturated or aromatic heterocyclic group having 1-2 hetero atom(s) selected from the group consisting of N, O, and S, each of which is optionally substituted by 1 or 2 substituent(s) each independently selected from the group A.

14. The compound of claim 13, wherein M is phenyl optionally having 1 or 2 hydroxyl or imidazol-5-yl group(s).

15. The compound of claim 1, which is represented by formula (I-III): 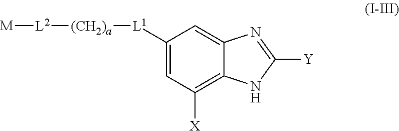
wherein
L¹ is —CONH— or a single bond;
L² is a single bond; and
M is amide or a 5-10 membered unsaturated or aromatic heterocyclic group having 1 or 2 hetero atom(s) selected from the group consisting of N, O, and S, optionally substituted by 1 or 2 substituent(s) each independently selected from the group A.

16. The compound of claim 1, which is represented by formula (I-IV): 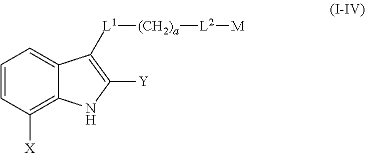
wherein
L¹ is —CONH—;
L² is a single bond; and
M is a 5-10 membered unsaturated or aromatic heterocyclic group having 1 or 2 hetero atom(s) selected from the group consisting of N, O, and S, optionally substituted by 1 or 2 substituent(s) each independently selected from the group A.

17. The compound of claim 1, which is represented by formula (I-V) or (I-VI): 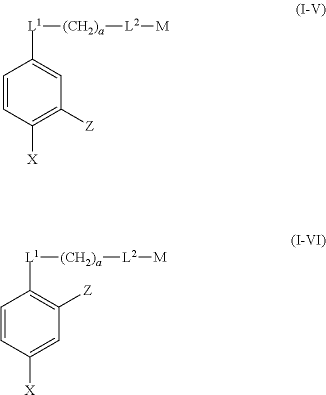
wherein
L¹ is —CONH—;
L² is a single bond; and
M is a 5-10 membered saturated, unsaturated or aromatic heterocyclic group having 1 or 2 hetero atom(s) selected from the group consisting of N, O, and S, optionally substituted by 1 or 2 substituent(s) each independently selected from the group A.

18. The compound of claim 1, wherein
L′ and L² are both a single bond;
M is carboxyl or amide; and
a is 0.

19. The compound of claim 1, wherein Y is thiophen-2-yl.

20. The compound of claim 1, wherein Y is furan-2-yl.

21. The compound of claim 1, wherein Y is phenyl.

22. The compound of claim 1, wherein Y is cyclopropyl.

23. The compound of claim 1, wherein Y is cyclopentyl.

24. The compound of claim 1, wherein Y is hydrogen.

25. The compound of claim 1, wherein Z is thiophen-2-ylcarbonylamino.

26. The compound of claim 1, which is selected from the group consisting of:
7-Hydroxy-N-[2-(phenylsulfonamido)ethyl]-2-(thiophen-2-yl)-1H-benzo[d]imidazole-4-carboxamide,
N-[2-(4-Chlorophenylsulfonamido)ethyl]-7-hydroxy-2-(thiophen-2-yl)-1H-benzo[d]imidazole-4-carboxamide,
N-[2-(5-Carbamoylpyridin-2-yloxy)ethyl]-7-hydroxy-2-(thiophen-2-yl)-1H-benzo[d]imidazole-4-carboxamide,
N-(2,4-Difluorobenzyl)-7-hydroxy-2-(thiophen-2-yl)-1H-benzo[d]imidazole-4-carboxamide,
7-Hydroxy-N-(4-sulfamoylbenzyl)-2-(thiophen-2-yl)-1H-benzo[d]imidazole-4-carboxamide,
7-Hydroxy-N-(3-methoxyphenethyl)-2-(thiophen-2-yl)-1H-benzo[d]imidazole-4-carboxamide,
N-[2-(5-Acetamidopyridin-2-ylamino)ethyl]-7-hydroxy-2-(thiophen-2-yl)-1H-benzo[d]imidazole-4-carboxamide,
N-[3-(1H-Imidazol-1-yl)propyl]-7-hydroxy-2-(thiophen-2-yl)-1H-benzo[d]imidazole-4-carboxamide,
7-Hydroxy-N-(4-sulfamoylphenethyl)-2-(thiophen-2-yl)-1H-benzo[d]imidazole-4-carboxamide,
7-Hydroxy-N-{2-[5-(methylsulfonamido)pyridin-2-ylamino]ethyl}-2-(thiophen-2-yl)-1H-benzo[d]imidazole-4-carboxamide,
7-Hydroxy-N-[2-(1-methyl-1H-imidazol-5-yl)ethyl]-2-(thiophen-2-yl)-1H-benzo[d]imidazole-4-carboxamide,
7-Hydroxy-N-[2-(4-nitrophenoxy)ethyl]-2-(thiophen-2-yl)-1H-benzo[d]imidazole-4-carboxamide,
Methyl 3-Hydroxy-2-[7-hydroxy-2-(thiophen-2-yl)-1H-benzo[d]imidazole-4-carboxamido]propanoate,
N-[2-(dimethylamino)ethyl]-7-hydroxy-2-(thiophen-2-yl)-1H-benzo[d]imidazole-4-carboxamide,
(S)-2-[7-Hydroxy-2-(thiophen-2-yl)-1H-benzo[d]imidazole-4-carboxamido]-3-(1H-indol-3-yl)propanoic Acid,
7-Hydroxy-2-(thiophen-2-yl)-N-[2-(thiophen-2-yl)ethyl]-1H-benzo[d]imidazole-4-carboxamide,
N-(2-Acetamidoethyl)-7-hydroxy-2-(thiophen-2-yl)-1H-benzo[d]imidazole-4-carboxamide,
N-3-(1H-Imidazol-2-yl)propyl)-]7-hydroxy-2-(thiophen-2-yl)-1H-benzo[d]imidazole-4-carboxamide,
Methyl 2-[7-Hydroxy-2-(thiophen-2-yl)-1H-benzo[d]imidazole-4-carboxamido]-3-(4-hydroxyphenyl)propanoate,
N-[2-(1H-Imidazol-2-yl)ethyl]-7-hydroxy-2-(thiophen-2-yl)-1H-benzo[d]imidazole-4-carboxamide,
2-(Furan-2-yl)-7-hydroxy-N-phenethyl-1H-benzo[d]imidazole-4-carboxamide,
2-(Furan-2-yl)-7-hydroxy-N-phenyl-1H-benzo[d]imidazole-4-carboxamide,
2-(Furan-2-yl)-7-hydroxy-N-[2-(1-methyl-1H-pyrrol-2-yl)ethyl]-1H-benzo[d]imidazole-4-carboxamide,
2-(Furan-2-yl)-7-hydroxy-N-(thiazol-2-yl)-1H-benzo[d]imidazole-4-carboxamide,
7-Hydroxy-N-[3-(5-oxo-4,5-dihydro-1H-pyrazol-4-yl)propyl]-2-(fran-2-yl)-1H-benzo[d]imidazole-4-carboxamide,
N-(2-(3,5-dimethylisoxazol-4-yl)ethyl)-2-(furan-2-yl)-7-hydroxy-1H-benzo[d]imidazole-4-carboxamide,
1-(2-Cyclopropyl-7-hydroxy-1H-benzo[d]imidazol-4-yl)-3-(4-hydroxyphenyl)urea,
N-(2-cyclopropyl-7-hydroxy-1H-benzo[d]imidazol-4-yl)-2-(4-hydroxyphenyl)acetamide,
2-Cyclopentyl-4-hydroxy-N-(4-hydroxyphenethyl)-1H-benzo[d]imidazole-7-carboxamide,
N-(4-Aminophenethyl)-2-cyclopentyl-4-hydroxy-1H-benzo[d]imidazole-7-carboxamide,
4-Hydroxy-N-(4-hydroxyphenethyl)-2-phenyl-1H-benzo[d]imidazole-7-carboxamide,
N-(4-Aminophenethyl)-4-hydroxy-2-phenyl-1H-benzo[d]imidazole-7-carboxamide, 4-Hydroxy-N-phenethyl-2-phenyl-1H-benzo[d]imidazole-7-carboxamide, 7-hydroxy-N-(3-methoxyphenethyl)-2-(thiophen-2-yl)-1H-benzo[d]imidazole-4-carboxamide,
N-(4-Fluorophenethyl)-7-hydroxy-2-(thiophen-2-yl)-1H-benzo[d]imidazole-4-carboxamide,
7-Hydroxy-N-[2-(pyridine-4-sulfonamido)ethyl]-2-(thiophen-2-yl)-1H-benzo[d]imidazole-4-carboxamide,
7-Hydroxy-N-[2-(4-methylbenzamido)ethyl]-2-(thiophen-2-yl)-1H-benzo[d]imidazole-4-carboxamide,
7-Hydroxy-N-(thiazol-2-yl)-2-(thiophen-2-yl)-1H-benzo[d]imidazole-4-carboxamide,
7-Hydroxy-2-(thiophen-2-yl)-N-[2-(5-(trifluoromethyl)pyridin-2-yloxy)ethyl]-1H-benzo[d]imidazole-4-carboxamide,
7-Hydroxy-N-[2-(pyridin-2-ylamino)ethyl]-2-(thiophen-2-yl)-1H-benzo[d]imidazole-4-carboxamide,
7-Hydroxy-N-(4-hydroxyphenethyl)-2-(thiophen-2-yl)-1H-benzo[d]imidazole-4-carboxamide,
7-Hydroxy-N-{2-[4-(4-methylphenylsulfonamido)phenoxy]ethyl}-2-(thiophen-2-yl)-1H-benzo[d]imidazole-4-carboxamide,
(S)-2-[7-Hydroxy-2-(thiophen-2-yl)-1H-benzo[d]imidazole-4-carboxamido]-3-(1H-imidazol-5-yl)propanoic Acid,
(S)-Methyl 2-[7-Hydroxy-2-(thiophen-2-yl)-1H-benzo[d]imidazole-4-carboxamido]-3-(1H-indol-3-yl)propanoate,
(S)-Methyl 2-[7-Hydroxy-2-(thiophen-2-yl)-1H-benzo[d]imidazole-4-carboxamido]-3-(1H-imidazol-5-yl)propanoate,
7-Hydroxy-N-[3-(2-hydroxyethylamino)propyl]-2-(thiophen-2-yl)-1H-benzo[d]imidazole-4-carboxamide,
N-[3-(Isopropylamino)propyl]-7-hydroxy-2-(thiophen-2-yl)-1H-benzo[d]imidazole-4-carboxamide,
(S)-7-Hydroxy-N-(1-hydroxy-3-phenylpropan-2-yl)-2-(thiophen-2-yl)-1H-benzo[d]imidazole-4-carboxamide,
7-Hydroxy-2-(thiophen-2-yl)-1H-benzo[d]imidazole-4-carboxylic acid,
(S)-7-Hydroxy-N-(1-hydroxy-3,3-dimethylbutan-2-yl)-2-(thiophen-2-yl)-1H-benzo[d]imidazole-4-carboxamide,
(R)-7-Hydroxy-N-[1-hydroxy-3-(1H-imidazol-4-yl)propan-2-yl]-2-(thiophen-2-yl)-1H-benzo[d]imidazole-4-carboxamide,
N-(3,4-Dihydroxybenzyl)-7-hydroxy-2-(thiophen-2-yl)-1H-benzo[d]imidazole-4-carboxamide,
2-(Furan-2-yl)-7-hydroxy-N-{2-[5-(4-methylphenylsulfonamido)pyridin-2-ylamino]ethyl}-1H-benzo[d]imidazole-4-carboxamide,
N-(3,4-Dihydroxyphenethyl)-2-(furan-2-yl)-7-hydroxy-1H-benzo[d]imidazole-4-carboxamide,
2-Cyclopropyl-N-(4-hydroxyphenyl)-4-hydroxy-1H-benzo[d]imidazole-7-carboxamide,
2-Cyclopropyl-4-hydroxy-N-(4-sulfamoylphenethyl)-1H-benzo[d]imidazole-7-carboxamide,
2-Cyclopropyl-N-(4-fluorophenethyl)-4-hydroxy-1H-benzo[d]imidazole-7-carboxamide,
2-Cyclopropyl-N-(2,3-dihydroxypropyl)-4-hydroxy-1H-benzo[d]imidazole-7-carboxamide,
2-Cyclopropyl-N-(2-(dimethylamino)ethyl)-4-hydroxy-1H-benzo[d]imidazole-7-carboxamide,
7-Hydroxy-2-(thiophen-2-yl)-1H-benzo[d]imidazole-5-carboxamide (Example No. 65),
N-[2-(1H-Imidazol-5-yl)ethyl]-7-hydroxy-2-(thiophen-2-yl)-1H-benzo[d]imidazole-5-carboxamide,
N-{5-[2-(1H-Imidazol-5-yl)ethylcarbamoyl]-2-hydroxyphenyl}thiophene-2-carboxamide,
N-[2-(1H-Imidazol-5-yl)ethyl]-7-fluoro-2-(thiophen-2-yl)-1H-benzo[d]imidazole-4-carboxamide,
N-[2-(1H-Imidazol-5-yl)ethyl]-7-hydroxy-1H-indole-3-carboxamide,
(E)-3-(1H-imidazol-5-yl)-N (7-hydroxy-2-(thiophen-2-yl)-1H-benzo[d]imidazol-4-yl)acrylamide,
N-(7-hydroxy-2-(thiophen-2-yl)-1H-benzo[d]imidazol-4-yl)-3-(1H-imidazol-5-yl)propanamide, and
N-(5-hydroxy-2-(piperidin-3-ylcarbamoyl)phenyl)thiophene-2-carboxamide.

27. A method for preparing a compound of claim 1 which comprises the steps of
reacting a carboxyalkyl substituted aniline derivative with nitrile in the presence of an acid;
cyclizing the intermediate amidine to obtain a benzimidazole derivative;
saponifying the carboxyalkyl of the benzimidazole derivative; and
forming an amide by either coupling the obtained carboxylic acid with an amine derivative, which may be further modified and extended after coupling, or
converting the carboxylic acid to an amine and then coupling with a carboxylic acid derivative, which may be further modified and extended after coupling, to obtain the amide compounds of claim 1.

28. A method for preparing a compound of claim 16 which comprises the steps of:
treating an indole derivative with trifluoroacetic acid anhydride to obtain a trifluoromethylketone;
hydrolyzing to the carboxylic acid; and
coupling the carboxylic acid with an amine derivative to obtain the compound of

29. A method of preparing a compound of claim 17 which comprises the steps of:
coupling a carboxyalkyl substituted aniline derivative with a carboxylic acid derivative;
hydrolyzing the carboxymethyl of the obtained amide to obtain a carboxylic acid; and
coupling the carboxylic acid with an amine derivative to obtain the compound of claim 17.

30. A pharmaceutical composition comprising at least one compound of claim 1 and a pharmaceutically acceptable carrier.

31. A pharmaceutical composition of claim 30 which is available for preventing or treating diseases selected from the group consisting of Alzheimer disease, mania, depression, migraine and type 2 diabetes.

32. A glycogen synthase kinase-3 Beta inhibitor comprising at least one compound of claim 1.
